(19) United States
(12) Patent Application Publication
(10) Pub. No.: US 2023/0042304 A1
(43) Pub. Date: Feb. 9, 2023

(54) Lymphatic Access, Drainage, And Shunting

(71) Applicant: NXT Biomedical, LLC, Irvine, CA (US)

(72) Inventors: Jeremy Koehler, Irvine, CA (US); Elliot Howard, Irvine, CA (US); Abubaker Khalifa, Irvine, CA (US); Joseph Passman, Irvine, CA (US); Glen Rabito, Irvine, CA (US); Stanton J. Rowe, Irvine, CA (US); Alexander Siegel, Irvine, CA (US); Robert S. Schwartz, Irvine, CA (US); Robert C. Taft, Irvine, CA (US)

(73) Assignee: NXT Biomedical, LLC, Irvine, CA (US)

(21) Appl. No.: 17/759,172

(22) PCT Filed: Jan. 15, 2021
(86) PCT No.: PCT/US2021/013684

§ 371 Date: Jul. 20, 2022

Related U.S. Application Data
(60) Provisional application No. 62/962,104, filed on Jan. 16, 2020.
(60) Provisional application No. 62/975,144, filed on Feb. 11, 2020.
(60) Provisional application No. 63/027,274, filed on May 19, 2020.

(51) Int. Cl. A61B 5/145; A61B 5/03; A61B 5/103; A61B 5/00; A61B 8/08

(52) U.S. Cl. CPC A61B 5/14503; A61B 5/036; A61B 5/1032; A61B 5/14507; A61B 5/14539; A61B 5/14546; A61B 5/418; A61B 5/4887; A61B 5/6852; A61B 5/6865; A61B 8/0841; A61B 2218/007; A61B 2576/02; A61F 2/82

(57) ABSTRACT

Several embodiments and methods are described for draining a lymphatic system for therapeutic purposes. The lymphatic draining can be performed by removal of fluid from the lymphatic system via a needle, a catheter, an access port, a reservoir, a shunt, or a combination of these devices. The drainage devices can be configured for use during only a single procedure or for reoccurring procedures.

### RELATED APPLICATIONS

This application claims benefit of and priority to U.S. Provisional Application Ser. No. 62/962,104 filed Jan. 16, 2020 entitled Chronic Volume Management, U.S. Provisional Application Ser. No. 62/975,144 filed Feb. 11, 2020 entitled Lymphatic Access, Drainage And Shunting, and U.S. Provisional Application Ser. No. 63/027,274 filed May 19, 2020 entitled Lymphatic Access, Drainage And Shunting, all of which are hereby incorporated herein by reference in their entireties.

### BACKGROUND OF THE INVENTION

Chronic and acute congestive heart failure (CHF) generally occurs when the heart is incapable of circulating an adequate blood supply to the body. This is typically due to inadequate cardiac output, which has many causes. In CHF decompensation fluids back up in a retrograde direction through the lungs and venous/lymphatic systems throughout the body, causing discomfort and organ dysfunction. Many diseases can impair the pumping efficiency of the heart to cause congestive heart failure, such as coronary artery disease, high blood pressure, and heart valve disorders.

In addition to fatigue, one of the prominent features of congestive heart failure is the retention of fluids within the body. Commonly, gravity causes the retained fluid to accumulate to the lower body, including the abdominal cavity, liver, and other organs, resulting in numerous related complications. Fluid restriction and a decrease in salt intake can be helpful to manage the fluid retention, but diuretic medications are the principal therapeutic option, including furosemide, bumetanide, and hydrochlorothiazide. Additionally, vasodilators and inotropes may also be used for treatment.

While diuretics can be helpful, they are also frequently toxic to the kidneys and if not used carefully can result in acute and/or chronic renal failure. This mandates careful medical management while in a hospital, consuming large amounts of time and resources. Hence, the ability to treat fluid retention from congestive heart failure without the need for toxic doses of diuretics would likely result in better patient outcomes at substantially less cost.

Fluid retention is not limited only to CHF. Conditions such as organ failure, cirrhosis, hepatitis, cancer, ascites, and infections can cause fluid buildup within the body.

In this regard, what is needed is an improved treatment option for fluid buildup in the body, whether that buildup is caused by CHF, cirrhosis, organ failure, cancer, infections, or other underlying diseases.

### SUMMARY OF THE INVENTION

The present invention is generally directed to different embodiments and methods of accessing, draining, and/or shunting a lymphatic system for therapeutic purposes.

Some embodiments include a catheter having one or more of the following features: anchoring features (e.g., radially enlarged shapes, one or more inflatable balloons, one or more expandable structures, or one or more hooks), curved or shaped distal ends, radially expandable distal ends, one or more magnets, one or more leaflet grasping mechanisms, one or more shaped drainage apertures, attached access ports, attached access septa, one or more filters, one or more valves, an attached reservoir, an attached fluid supply, an attached pump, a stylet, one or more sensors, and/or a perfusion passage.

Some embodiments include an access port having one or more of the following features: a housing configured for subcutaneous implantation, a housing configured for external connection, a sealing mechanism within the port housing, a sealing mechanism having one or more layers, one or more filters, one or more tactile or visualization markers, a single outlet, two outlets, a dome shaped sealing member, a conical shape, one or more valves, one or more balloons, a coupling device, a connected reservoir, a connected fluid supply, and/or one or more sensors.

Some embodiments include a fluid reservoir having one or more of the following features: one or more valves, one or more filters, one or more sensors, one or more fill sensors, one or more pressure sensors, one or more flow sensors, a septum, and/or one or more anchoring mechanisms.

Some embodiments include a shunt having one or more of the following features: one or more anchoring mechanisms (e.g., radially expandable ends, terminal hooks, etc.), one or more valves, one or more magnetically actuated valves, one or more electronically actuated valves, one or more sensors, and/or a reservoir.

Some embodiments include a method of access including one or more of the following: direct lymphatic system access, lymphatic system access via a venous vessel, lymphatic access via needle, lymphatic access via catheter, lymphatic access via shunt, lymphatic access via an access port, lymphatic access via a reservoir, lymphatic drainage during a single procedure, lymphatic drainage during several different procedures, and/or lymphatic pumping.

Some embodiments include a system for accessing, re-accessing, and draining lymphatic fluid including one or more of the following elements: one or more catheters, one or more needles, one or more ports, one or more sensors, one or more reservoirs, one of more markers, one or more filters, one or more valves, one or more stylets, and/or a suction source.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a view of anatomy in and around a thoracic duct.

FIG. 2 is a view of a needle entry locations to a lymphatic structure.

FIG. 3 is a view of a needle entry location to a thoracic duct.

FIG. 4 is a view of a catheter within a thoracic duct.

FIG. 5 is a view of a catheter within a thoracic duct.

FIG. 6 is a view of a catheter within a cisterna chyli.

FIG. 7 is a view of a needle.

FIG. 8 is a view of a needle within a thoracic duct.

FIG. 9A is a view of an ultrasound transceiver and needle adapter.

FIG. 9B is a view of a needle adapter.

FIG. 10 is a view of a catheter within a thoracic duct.

FIGS. 11A, 11B, 11C, and 11D are views of different shapes of distal ends of catheters.

FIG. 12 is a view of an anchoring device for a catheter.

FIG. 13 is a view of an anchoring device for a catheter.

FIG. 14 is a view of an anchoring device for a catheter.

FIG. 15 is a view of an anchoring device for a catheter.

FIG. 16 is a view of an anchoring device for a catheter.

FIGS. 17A and 17B are views of an anchoring device for a catheter.

FIG. 18 is a view of an anchoring device for a catheter.

FIG. 19 is a view of an anchoring device for a catheter.

FIG. 20 is a view of an anchoring device for a catheter.

FIG. 21A is a view of a distal end of a catheter.

FIGS. 21B and 21C are cross sectional views of the catheter in FIG. 21A.

FIGS. 22A and 22B are views of a closure device.

FIGS. 23A and 23B are views of a closure device.

FIG. 24 is a view of a catheter within a thoracic duct.

FIG. 25 is a view of a catheter within a thoracic duct.

FIG. 26 is a view of a catheter within a thoracic duct.

FIG. 27 is a view of a catheter within a thoracic duct.

FIG. 28 is a view of a catheter within a cistern chyli.

FIG. 29 is a view of an angle of the opening of a thoracic duct.

FIG. 30 is a view of a guidewire approaching the opening of a thoracic duct.

FIG. 31 is a view of a catheter with a curved distal end.

FIG. 32 is a view of a catheter with a curved distal end.

FIG. 33 is a view of a catheter with an anchoring mechanism.

FIG. 34 is a view of a catheter with an anchoring mechanism.

FIG. 35 is a view of a catheter and a stent within a thoracic duct.

FIG. 36 is a view of a catheter with one or more magnets.

FIG. 37 is a view of a catheter with one or more hooks.

FIG. 38 is a view of a catheter with a control wire.

FIG. 39 is a view of a catheter with one or more anchoring hooks.

FIG. 40 is a view of a catheter with one or more grasping mechanisms.

FIG. 41 is a view of a catheter with an anchoring balloon.

FIG. 42 is a view of a catheter with an anchoring balloon.

FIG. 43 is a view of a catheter with an expandable anchoring mesh.

FIG. 44 is a view of a catheter with an expandable anchoring mesh.

FIG. 45 is a view of a catheter with a plurality of anchoring hooks.

FIG. 46 is a view of a catheter with one or more magnets.

FIG. 47 is a view of a catheter with one or more magnets.

FIG. 48 is a view of a catheter with one or more anchoring balloons.

FIG. 49 is a view of a catheter with one or more anchoring balloons.

FIG. 50 is a view of a catheter within a thoracic duct.

FIG. 51 is a view of a catheter within a thoracic duct.

FIG. 52 is a view of a catheter within a thoracic duct.

FIG. 53 is a view of a catheter within a thoracic duct.

FIG. 54 is a view of a catheter within a thoracic duct.

FIG. 55 is a view of a catheter with one or more drainage apertures.

FIG. 56 is a view of a catheter with one or more drainage apertures.

FIG. 57 is a view of a catheter with one or more drainage apertures.

FIG. 58 is a view of a catheter with one or more drainage apertures.

FIGS. 59A, 59B, 59C, 59D, 59E, and 59F illustrate views of cross sectional shapes of a catheter.

FIG. 60 is a view of a catheter with one or more balloons.

FIG. 61 is a view of a catheter with one or more balloons.

FIG. 62 is a view of a catheter with an expandable mesh structure.

FIG. 63 is a view of a catheter with an expandable mesh structure.

FIG. 64 is a view of a catheter within a thoracic duct.

FIG. 65 is a view of a stylet.

FIG. 66 is a view of a stylet.

FIG. 67 is a view of a stylet.

FIG. 68 is a view of a catheter with one or more sensors.

FIG. 69 is a view of a catheter with one or more sensors.

FIGS. 70A and 70B are views of a catheter with multiple lumens.

FIGS. 71A and 70B are views of a catheter with multiple lumens.

FIG. 72 is a view of a catheter with a balloon.

FIG. 73 is a view of a catheter with a balloon.

FIG. 74 is a view of a catheter with one or more filters.

FIGS. 75A and 75B are views of a catheter with an expandable mesh structure.

FIGS. 76A and 76B are views of a catheter with one or more perfusion balloons.

FIG. 77 is a view of a catheter with a plurality of drainage ports.

FIG. 78 is a view of a catheter with a plurality of drainage ports.

FIG. 79 is a view of a catheter with a port external to the patient.

FIG. 80 is a view of a catheter with a subdermal port.

FIG. 81 is a view of an incision near a thoracic duct.

FIG. 82 is a view of a catheter and access port.

FIG. 83 is a view of an access port.

FIG. 84 is a view of a catheter with a flange.

FIG. 85 is a view of an access port.

FIG. 86 is a view of a catheter with an external access port.

FIG. 87 is a view of a catheter with an external access port.

FIG. 88 is a view of an access port.

FIG. 89 is a view of an access port.

FIG. 90 is a view of an access port.

FIG. 91 is a view of an access port.

FIG. 92 is a view of an access port.

FIG. 93 is a view of a catheter with an access port.

FIG. 94 is a view of an access port.

FIG. 95 is a view of an access port.

FIG. 96 is a view of a catheter with a bypass lumen.

FIG. 97 is a view of an access port.

FIG. 98 is a view of an access port.

FIG. 99 is a view of an access port.

FIG. 100 is a view of an access port.

FIG. 101 is a view of an access port.

FIG. 102 is a view of a support structure for an access port.

FIG. 103 is a view of an access port.

FIG. 104 is a view of an access port.

FIG. 105 is a view of an access port.

FIG. 106 is a view of an access port attached to a catheter.

FIG. 107 is a view of an access port attached to a catheter.

FIG. 108 is a view of an access port.

FIG. 109 is a view of an access port attached to a catheter.

FIG. 110 is a view of an access port attached to a catheter.

FIG. 111 is a view of a valve.

FIG. 112 is a view of a valve.

FIG. 113 is a view of an access port.

FIG. 114 is a view of an access port.

FIG. 115 is a view of an access port and a catheter.

FIG. 116 is a view of an access port and a catheter.

FIGS. 117A, 117B, 117C, 117D, 117E, 117F are views of various stylet shapes.

FIG. 118 is a view of an access port.

FIG. 119 is a view of an access port.

FIG. 120 is a view of an access port.

FIGS. 121A and 121B are views of an access port.

FIG. 122 is a view of an access port connector.

FIG. 123 is a view of an access port.

FIG. 124 is a view of a catheter, reservoir, and fluid source.

FIG. 125 is a view of a catheter, reservoir, and fluid source.

FIG. 126 is a view of an access port.

FIG. 127 is a view of an access port.

FIG. 128 is a view of a subcutaneous access port.

FIG. 129 is a view of an access port external of a patient's skin.

FIG. 130 is a view of a reservoir.

FIG. 131 is a view of a reservoir.

FIG. 132 is a view of a subcutaneous reservoir.

FIG. 133 is a view of a reservoir.

FIG. 134 is a view of a reservoir.

FIG. 135 is a view of a reservoir.

FIG. 136 is a view of a reservoir.

FIG. 137 is a view of a reservoir.

FIG. 138 is a view of a reservoir.

FIG. 139 is a view of a reservoir.

FIG. 140 is a view of an externally located reservoir.

FIG. 141 is a view of a reservoir.

FIG. 142 is a view of a reservoir with a fill sensor.

FIG. 143 is a view of a reservoir with a fill sensor.

FIG. 144 is a view of a reservoir with a fill sensor.

FIG. 145 is a view of a reservoir with a fill sensor.

FIG. 146 is a view of a reservoir.

FIG. 147 is a view of a reservoir.

FIG. 148 is a view of a reservoir.

FIG. 149 is a view of a subcutaneous reservoir.

FIG. 150 is a view of a reservoir.

FIG. 151 is a view of a reservoir.

FIG. 152 is a view of a reservoir.

FIG. 153 is a view of a plurality of markers.

FIG. 154 is a view of a stent marker.

FIG. 155 is a view of a stent marker.

FIG. 156 is a view of a stent marker.

FIG. 157 is a view of a stent marker.

FIG. 158 is a view of a marker.

FIG. 159 is a view of a marker.

FIG. 160 is a view of a marker near a thoracic duct.

FIG. 161 is a view of a first and second catheter.

FIG. 162 is a view of a first and second catheter.

FIG. 163 is a view of an expandable catheter.

FIG. 164 is a view of an expandable catheter.

FIG. 165 is a view of an implanted catheter.

FIG. 166 is a view of an implanted catheter.

FIG. 167 is a view of an implanted catheter.

FIG. 168 is a view of an implanted catheter.

FIG. 169 is a view of a catheter with magnetic connector.

FIG. 170 is a view of a catheter with a magnetic connector.

FIG. 171 is a view of two catheters with magnetic connectors.

FIG. 172 is a view of an implanted catheter.

FIG. 173 is a view of a marker and needle within a patient.

FIG. 174 is a view of a marker and needle within a patient.

FIG. 175 is a view of a catheter within a patient.

FIG. 176 is a view of a catheter within a patient.

FIG. 177 is a view of a snare catheter within a patient.

FIG. 178 is a view of a catheter within a patient.

FIG. 179 is a view of a catheter within a patient.

FIG. 180 is a view of a catheter within a patient.

FIG. 181 is a view of a catheter within a patient.

FIG. 182 is a view of a catheter within a patient.

FIG. 183 is a view of a catheter within a patient.

FIG. 184 is a view of a fluid filtration system.

FIG. 185 is a view of an access path to a lymphatic system.

FIG. 186 is a view of a shunt.

FIG. 187 is a view of a shunt.

FIG. 188 is a view of a shunt.

FIG. 189 is a view of a shunt.

FIG. 190 is a view of a shunt.

FIG. 191 is a view of a shunt.

FIG. 192 is a view of a shunt.

FIG. 193 is a view of a shunt with a reservoir.

FIG. 194 is a view of a balloon catheter.

FIG. 195 is a view of a balloon catheter.

FIG. 196 is a view of an inflatable cuff.

FIG. 197 is a view of an inflatable cuff.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present specification is directed to several different embodiments and methods related to drainage of the lymphatic system. While aspects of each embodiment and method are presented individually for clarity, the intent of the specification is that any embodiments and methods can be combined and used interchangeably with each other without limitation, unless specified otherwise. Hence, while embodiments with specific combinations of steps or features may not be described, such combinations are contemplated and intended to be encompassed by the present specification.

This specification is directed to several different treatment devices and methods of use. Some of these devices and methods may be performed via direct access to a lymphatic structure (e.g., any part of the lymphatic system, such as portion of the thoracic duct, right thoracic duct, cisterna chyli, lymph node, or collecting lymphatic structure). Any methods and embodiments described elsewhere in this specification can be used with these treatment techniques unless specifically indicated otherwise. Also, additional explanation and embodiments that can be used with and according to the those in the present specification can be found in U.S. Pub. No. 2020/0054867 entitled System And Method For Treatment Via Bodily Drainage Or Injection, the contents of which are hereby incorporated by reference.

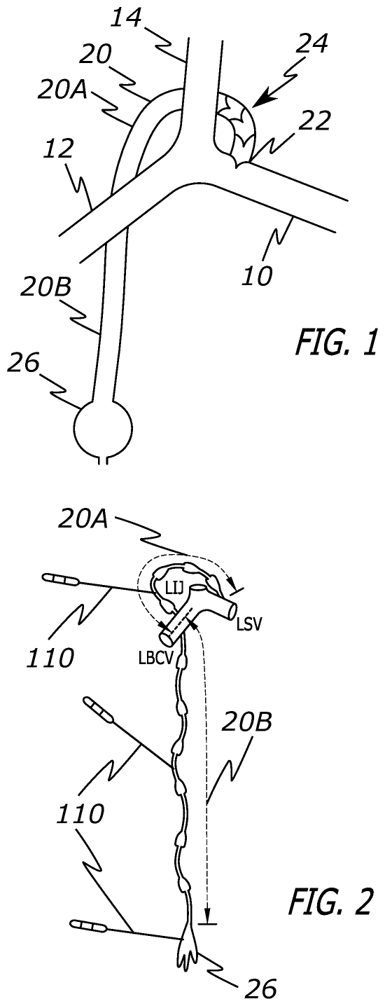

FIG. 1 illustrates several locations within the body that may be of particular relevance when performing the direct access techniques that follow. As seen in FIG. 1, the thoracic duct 20 has an opening into the left subclavian vein 10, near the left internal jugular 14 and right brachiocephalic vein 12. While the thoracic duct is shown with an opening into the left subclavian vein in FIG. 1, in some patients it may have an opening into the left internal jugular vein, an opening into the junction between the left subclavian vein, external jugular vein, vertebral vein, and the internal jugular vein, or a plurality of branches at its distal end that may terminate into one or more of the aforementioned veins or confluences thereof. The thoracic duct 20 includes a plurality of internal valves 24, as well as a terminal valve 22 at the interface or annulus with the left subclavian vein 10. The thoracic duct 20 includes an upper cervical portion 20A and a lower thoracic portion 20B that is connected to the cisterna chyli 26.

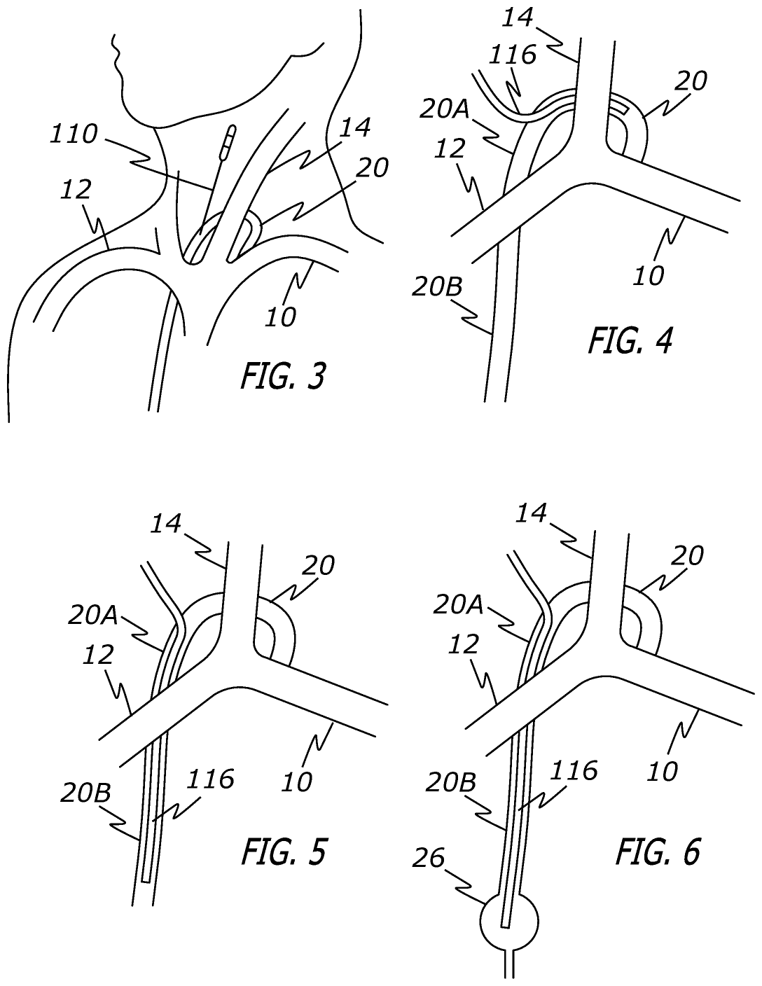

In one example method, an access device such as a needle 110 is advanced through the skin and directly into a lymphatic structure of a patient in order to remove lymphatic fluid from the structure. For example, the needle 110 may be advanced through the skin of a patient and directly into a lymph node, a thoracic duct, a right thoracic duct, a renal lymphatic vessel, and/or the cisterna chyli. The thoracic duct may be accessed in its cervical portion (e.g. above the brachiocephalic vein) or in its thoracic potion (e.g. below the brachiocephalic vein). For simplicity, the thoracic duct 20 will be used as an example of a lymphatic structure; however, any lymphatic structure may be used without deviating from the present invention. FIG. 2 illustrates three such example access sites in which the needle 110 is advanced into the upper cervical portion 20A (also shown in FIG. 3), the lower thoracic portion 20B, or the cisterna chyli 26 (or even combinations thereof).

In order to confirm the thoracic duct 20 is successfully accessed, suction is applied to the needle 110 to withdraw fluid from the target structure into the needle 110. The fluid may then be identified by one or more properties including but not limited to color, pH, viscosity, impedance, salinity, and presence or absence of constituents such as red blood cells.

In another method, a needle 110 with a sensor 124 at its distal tip is advanced into a thoracic duct 20 in order to confirm that the thoracic duct 20 was successfully accessed. The sensor 124 may directly measure one or more physical properties of the fluid including but not limited to color, pH, viscosity, impedance, and salinity, or one or more properties of the thoracic duct 20 including but not limited to pressure, orientation, compliance, presence of valves, contractile motion, and size.

In one method, a needle 110 is advanced into a thoracic duct and, in order to confirm the thoracic duct was successfully accessed, a contrast agent such as lipiodol is injected through the needle and into the target structure. X-Ray or fluoroscopy is then used to directly visualize the internal anatomy of the structure and determine whether the correct structure was successfully accessed.

In another method, a contrast agent such as lipiodol may be injected into a lymph node and allowed to spread throughout the lymphatic network. The lymphatic network may then be visualized using X-ray or fluoroscopy to determine a suitable location to access the desired lymphatic structure. For example, if a thoracic duct 20 contains numerous small branches in the cervical portion 20A (plexiform anatomy), an access device may be inserted into the thoracic portion 20B or into the cisterna chyli 26. Alternatively, instead of X-ray or fluoroscopy, ultrasound may alternately be used to identify the anatomy of the lymphatic network to identify a desired access location.

After confirmation that the thoracic duct has been accessed, a guidewire may be advanced through the needle 110 and into the thoracic duct 20. The needle 110 may be removed and a catheter 116 with one or more lumens may be advanced over the guidewire. Once the catheter 116 is in place, lymphatic fluid may be drained through the one or more lumens within the catheter's body. Depending on the access site and direction, a distal end of the catheter 116 may be advanced in an antegrade or retrograde direction to a location within the upper cervical portion 20A as seen in FIG. 4 (e.g. between the final two, three, or four valves 24), in the lower thoracic portion 20B of the thoracic duct 20 as seen in FIG. 5, or in the cisterna chyli 26 as seen in FIG. 6. The catheter 116 may, for example, be a length within a range inclusive of about 5 centimeters (cm) and 150 cm (5 cm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 100 cm, 105 cm, 110 cm, 115 cm, 120 cm, 125 cm, 130 cm, 135 cm, 140 cm, 145 cm, or 150 cm).

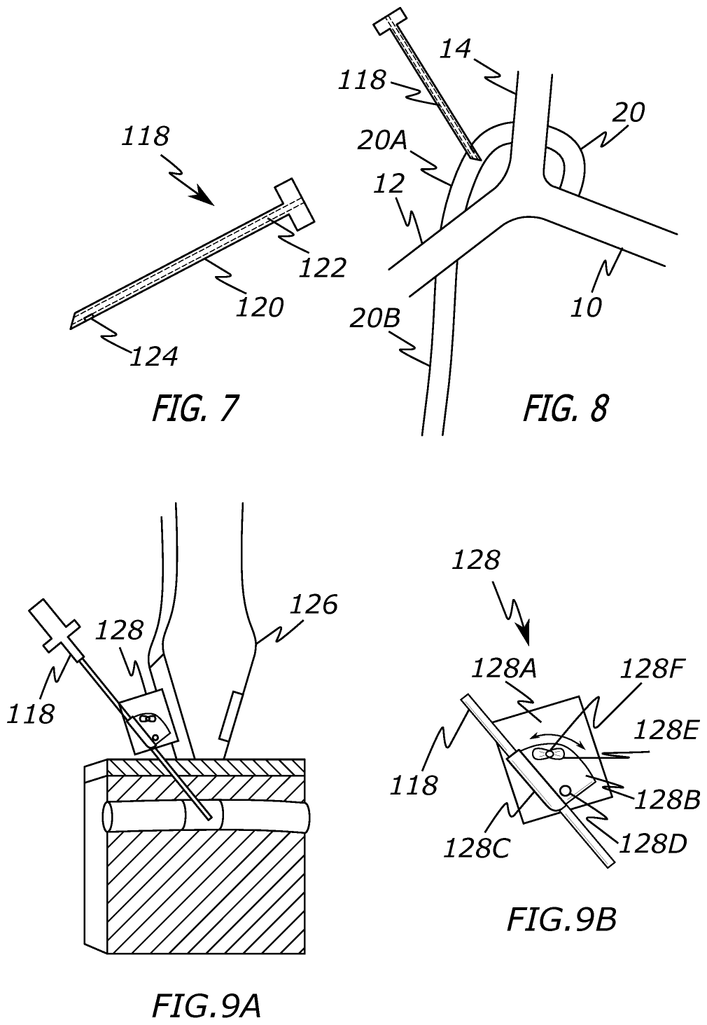

In order to guide an access device to its intended location, imaging techniques such as ultrasound, CT, or MRI may be used with or without the aid of contrast agents. For example, the target structure may be identified using an ultrasound probe based on relevant anatomical landmarks such as a vein or confluence of veins or physiologic features such as flow patterns of fluid inside the structure, motion of the structure, or presence of one or more valves within the structure. In one example, a needle device 118 includes an elongated needle portion 120 and a passage therethrough (FIG. 7), which may be advanced through the skin and into the field of view of an ultrasound probe (FIG. 8). The needle 118 includes echogenic properties (e.g., composed of echogenic material or that includes an echogenic member on the tip of the needle (e.g., a band, coating, sleeve, layer, etc.) so that it can be visualized during the procedure. Optionally, the tip of the needle may also include a sensor 124, as previously described (e.g., for color, pH, viscosity, impedance, and salinity). The tip of the needle portion 120 may be tracked in real-time as the operator advances into the target structure.

Additionally, a needle guide 128 may be coupled to the ultrasound probe 126 (e.g., to the body or cover of the probe 126) to provide a needle trajectory to guide the needle into the desired structure. This can be achieved, for example, by overlaying the trajectory of the needle device 118 into the field of view of the ultrasound probe 126 as shown in FIGS. 9A and 9B. The needle guide 128 may be adjustable to change the angle and depth of the projected needle path in the tissue. This can be achieved, in one example, with a pivoting hinge mechanism for angle adjustment purposes and with an adjustable needle holding member that can be locked at various angles.

More specifically, the needle guide 128 may be comprised of a base portion 128A that attaches to an ultrasound probe 126, for example as a unitary part of the probe's housing or as a clip-on attachment mechanism. As best seen in FIG. 9B, a pivoting plate 128B is attached to the base portion 128A via a pivot connection 128D, which allows the plate 128B to pivot or rotate about the pivot connection 128D. The pivoting plate 128B includes a tubular lumen 128C that is angled downward (i.e., towards the distal end of the ultrasound probe 126) and is sized to hold and/or guide the direction of the needle device 118 as it passes through. In that respect, the user can adjust the angle or trajectory of the needle device 118 relative to the ultrasound probe 126 by pivoting the pivoting plate 128B.

A locking mechanism may also be included to lock the position of the pivoting plate 128B relative to the base portion 128A. For example, this may include a bolt 128F and wingnut 128E that passes through both the pivoting plate 128B and the base portion 128A to frictionally engage each component.

The needle guide 128 may also include a mechanism that senses the angular position of the pivoting plate 128B to determine the angle and/or trajectory of the needle device 118. For example, a rotary encoder may be included to sense the position of the pivoting plate 128B and communicate that angle data to a control unit of the ultrasound probe. With that data, the ultrasound monitor can overlay an estimated trajectory of the needle device 118 prior to being inserted into the patient, thereby providing the physician with a better idea of exactly which internal structures the needle device 118 will pass into. Alternately, the needle guide 128 may include a plurality of indicia corresponding to a position of the pivoting plate 128B. Hence, an angular position can be visually determined by the physician and entered into the ultrasound monitor or equipment.

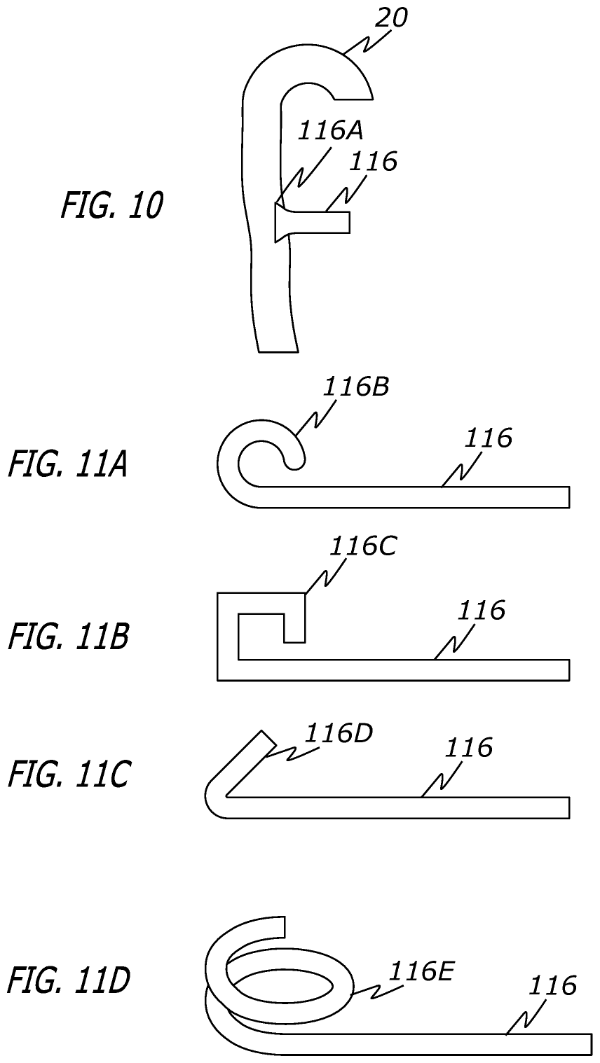

Additionally, in order to maintain the position of the catheter 116 within the thoracic duct 20, or other lymphatic structure, one or more fixation mechanisms may be employed. This could be advantageous to prevent migration of the catheter 116 due to contractile motion of the thoracic duct, motion of the lungs during breathing, or other motion of the patient's body. FIG. 10 illustrates a first example of a fixation mechanism in which the distal end of the catheter is expandable to a larger size than the entry hole in the thoracic duct. The catheter tip 116A may be comprised of a self-expanding material, such as nitinol, or expanded to a final size by another device such as a balloon.

In one example, a self-expanding catheter tip 116A may be comprised of a unitary nitinol wire (or similar shape-memory material) formed or shape-set into a spiral shape with a distal diameter greater than a proximal diameter. The nitinol wire may be attached to the catheter 116 by welding means, adhesive means, or press-fit means, Alternatively, a plurality of nitinol wires could be used and or shape-set into a funnel shape with a distal diameter greater than a proximal diameter. Either embodiment of catheter tip 116A disclosed above may be collapsed and placed inside an exterior sheath (not shown) prior to insertion into the lymphatic structure. Once the catheter tip 116A and sheath are inserted into the lymphatic structure, the sheath is retracted, and the nitinol catheter tip 116A is expanded to help maintain the distal end in the lymphatic structure.

Alternatively, an expandable catheter tip 116A may be comprised at least partially of steel, stainless steel, cobalt, chromium, or combination thereof. The catheter tip 116A may be formed from laser-cut tubing with struts and spaces such as is used in expandable metal stents. The struts and spaces may be designed such that, following expansion from within, the structure expands such that it has a distal diameter greater than a proximal diameter. For example, the spaces may be smaller and/or less numerous in the proximal section and larger and/or more numerous in the distal section to reduce the hoop strength of the structure in the distal section relative to the proximal section to achieve the desired shape profile. In use, the catheter 116 may be inserted into the lymphatic structure and a second balloon catheter may be introduced through the lumen of the catheter 116. The balloon catheter is then expanded at least partially within the catheter tip 116A to at least partially expand the catheter tip 116A into the desired shape to help maintain the distal end in the lymphatic structure.

The distal end of the catheter 116 may alternately be shape-set to form a nonlinear, three-dimensional shape when unconstrained to help maintain the distal end of the catheter 116 within the lymphatic structure. The catheter's distal tip may be shape set in a variety of shapes including but not limited to a circular shape, a rectangular shape, a spiral shape, and an angled shape. For example, FIG. 11A illustrates a curved or circular shape 116B, FIG. 11B illustrates a square or rectangular shape 116C, FIG. 11C illustrates a bent or angled shape 116D, and FIG. 11D illustrates a coiled or circular shape 116E.

During insertion, the catheter ends are maintained in a substantially linear form by an internal straightening member that extends to the catheter's distal end (e.g., a rigid wire positioned through a lumen in the catheter 116). After the distal end of the catheter 116 has been advanced into the lymphatic structure, the straightening member may be at least partially removed to allow the catheter 116 to bend/reposition to take its set shape, preventing the distal end from pulling out and thereby maintain it within in the lymphatic structure.

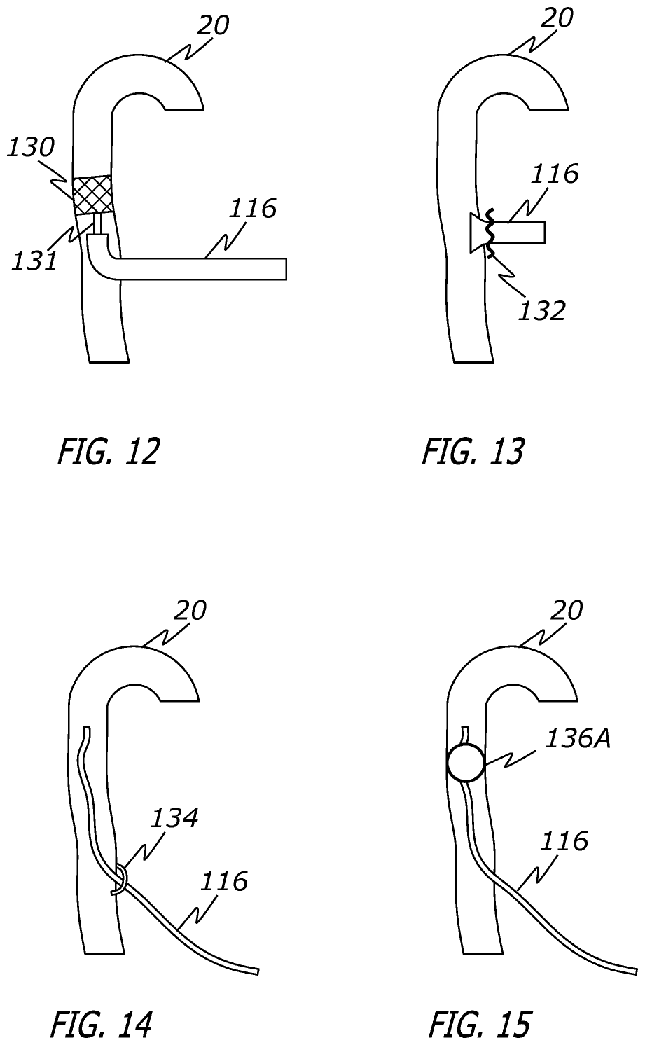

FIG. 12 illustrates another embodiment for anchoring or securing a catheter 116 within a lymphatic structure. Specifically, a stent 130 may be deployed within the lymphatic vessel and an attachment structure 131 on the distal end of the catheter may attach to the stent 130. The attachment structure 131 may include hooks, magnets, or wires. In one embodiment, the stent 120 is delivered via a first delivery catheter, the delivery catheter is removed, and the drainage catheter 116 is then advanced into the lymphatic structure and connected to its distal end via the attachment structure 131. In another embodiment, the distal end of the drainage catheter 116 may have a stent 130 that can be delivered from the drainage catheter 116 and remain connected via the attachment structure 131. The attachment structure 131 may include a mechanism to allow the physician to selectively release attachment to the stent 130.

FIG. 13 illustrates another embodiment in which the catheter 116 is attached to the lymphatic vessel using adhesive 132. The catheter 116 may have a rough outer surface finish, a flared distal end, and/or undercut features to enhance engagement of the adhesive to the catheter. The distal end of the catheter 116 is advanced into the lymphatic structure and the adhesive is applied at least partially in a circumferential manner around the interface between the lymphatic structure and the catheter 116. The adhesive may be positioned around the interface between the lymphatic structure and the catheter 116 by a needle that is advanced through the tissue to the interface location. Alternatively, the catheter 116 may be comprised of one or more lumens through which adhesive is advanced, in addition to a central lumen. In one embodiment, two or more lumens for adhesive transfer may be spaced radially around the central lumen. The catheter 116 may be advanced until it is in contact with the lymphatic structure and adhesive is dispensed through the lumens. The catheter may be rotated substantially about its central axis to achieve application of the adhesive in at least a partially circumferential manner.

FIG. 14 illustrates another embodiment in which the catheter 116 may be attached to the lymphatic vessel or nearby tissue using a clip 134 made from any suitable material including nitinol, steel, or deformable polymer. A clip 134 may be inserted and applied manually by a surgeon. Alternatively, a clip 134 may be loaded into a clip applier (not shown) and advanced to the interface between the lymphatic vessel and the catheter 116. The clip 134 may then be deployed from the clip applier and attach the catheter 116 to the lymphatic vessel or nearby tissue.

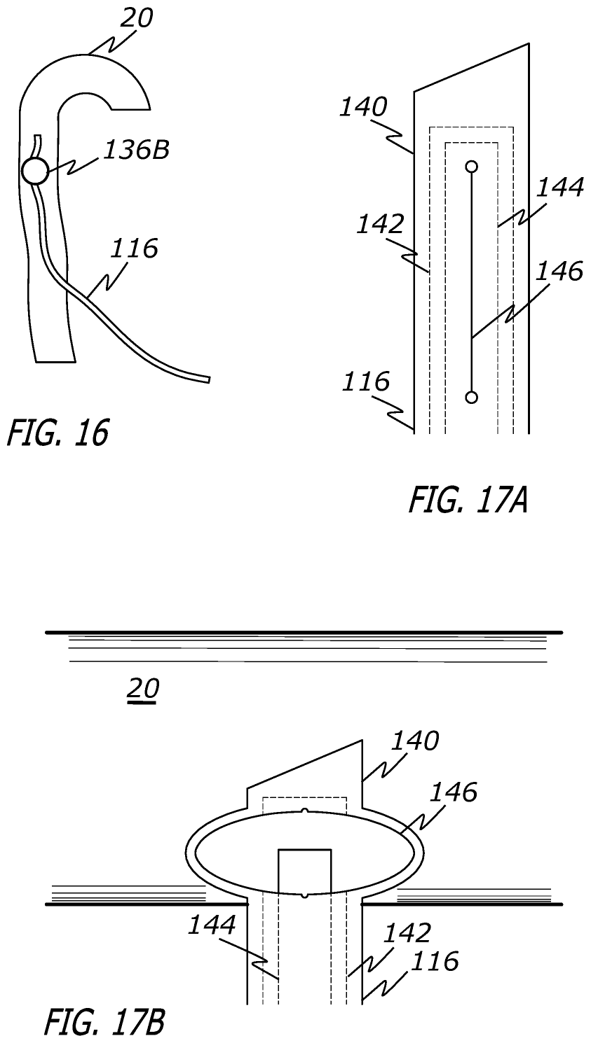

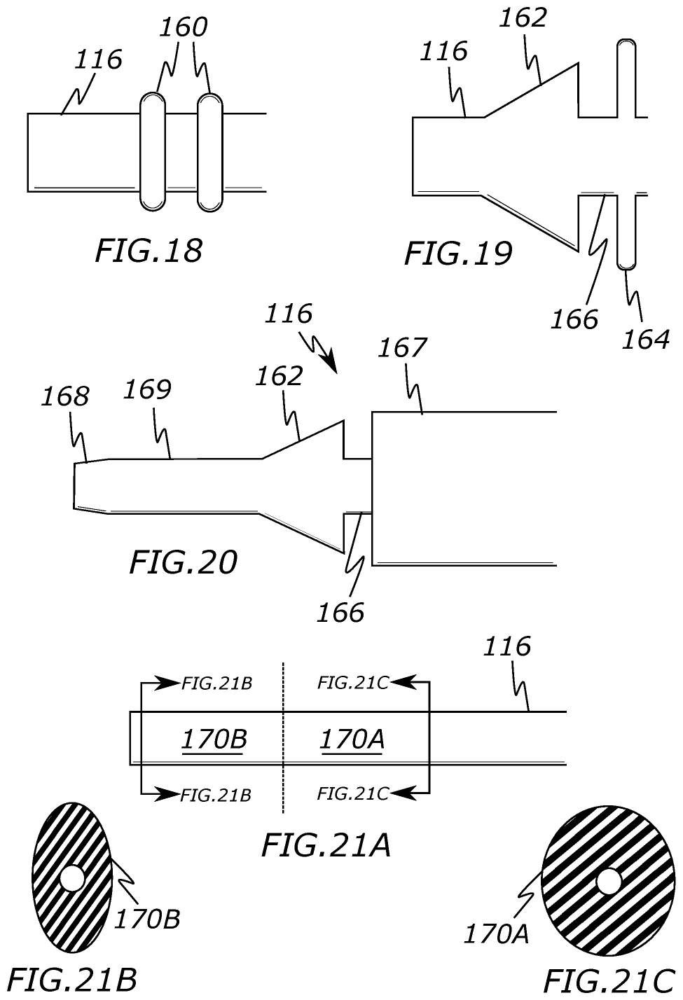

FIGS. 15 and 16 illustrate another embodiment in which the catheter 116 includes a balloon 136N136B that is inflated to a size larger than the entry hole in the lymphatic structure (e.g., thoracic duct 20) to maintain its position. The catheter 116 may have both an inflation lumen for inflating/deflating the balloon 136 and a drainage lumen for draining the lymphatic fluid. The balloon 136A may be expanded to seal against the wall of the thoracic duct 20 to maximize the flow rate of fluid through the drainage catheter lumen, as seen in FIG. 15. Alternatively, the balloon 136B may be expanded to a size smaller than the diameter of the lymphatic structure to allow lymphatic fluid to flow around the balloon 136, as seen in FIG. 16. Allowing some native lymphatic fluid may be advantageous to avoid removing all of the proteins, white blood cells, leukocytes, and electrolytes present in lymphatic fluid. Alternatively, the balloon may contain pass-through or perfusion passage to permit flow in the expanded state as described elsewhere in this application.

FIGS. 17A and 17B illustrate another embodiment in which a tip 140 of the catheter 116 may be biased or otherwise controllable to expand to a larger size. For example, the tip 140 includes an axially aligned longitudinal slit 146 that is positioned along at least a portion of the outwardly biased tip 140. As the tip 140 expands, it causes the slit 146 to open into the lumen 142 within the tip 140 and causes the portions of the catheter adjacent the slip 146 to bow or bend away from the axis of the catheter 116. Alternately, the lumen 142 may extend through the distal tip 140 when the slit 146 is open. The lumen may further include an inner cannula or tubular portion 144 that is connected to a drainage device. Optionally, the tubular portion 144 can move longitudinally so that when distally positioned, it maintains the tip 140 in a radially compressed position (FIG. 17A) and when moved proximally it releases the tip to allow for radial expansion (FIG. 17B). Lymph fluid can be pulled through the open slit 146 and the larger radial diameter can help anchor the catheter tip 140.

In other embodiments, the drainage catheter 116 may include a repositionable securing mechanism that are configured to provide at one and preferably at least two longitudinally movable flanged portions 160 (e.g., rings, flanges, lips, or similar raised structures) at locations on the outer surface of the distal tip where the catheter 116 enters the lumen of the lymphatic structure. By allowing the flanged portions 160 to be longitudinally movable, the physician can determine the desired length of catheter 116 that can be advanced into the lymphatic structure before being stopped by the flanged portions 160. The distal flanged portion 160 can be advanced into the lymphatic structure and the proximal flanged portion 160 can remain outside the lymphatic structure.

In one example, the flanged portion 160 is composed of flexible circular O-rings that are slidably placed and movable in a desired location near the distal tip of the catheter 116. The inner diameter of the O-rings 160 may be smaller than the outer diameter of the catheter to achieve a friction fit. Alternatively, the flanged portions 160 may be threaded nuts that engage with a mating thread on the outer surface of the catheter 116, allowing the nuts to be rotated to cause longitudinal displacement along the catheter 116. In another example, the repositionable flanged portions 160 may be balloons that are inflated to maintain a desired location by squeezing the catheter and deflated to be re-positioned. Optionally, the catheter 116 may include several different balloons as flanged portions 160, allowing the physician to choose which balloons are inflated based on a desired distance the catheter 116 should advance into the lymphatic structure.

In another embodiment seen in FIG. 19, a distal end of the catheter 116 may be comprised of a taper 162, flange 164 and a recess 166 separating the two structures. The taper 162 can more easily pass through an aperture in a lymphatic structure causing its wall to stretch, while the perpendicular backside of the taper 162 and the flange 164 help maintain the wall of the lymphatic structure within the recess 166. If the length of catheter from the taper 162 to the distal end is longer than a physician desires or is too long to accommodate a given patient's anatomy, the distal end of the catheter may be cut to the desired length to accommodate a wide variety of anatomical situations. However, the taper 162 and flange 164 can also be longitudinally movable as previously described.

FIG. 20 illustrates another embodiment that is similar to the previously described embodiment of FIG. 19, including a taper 162 and a recess 166. However, instead of a proximal flange, the catheter 116 includes an outer sheath 167 that can move relative to an inner catheter portion 169, which includes the taper 162 and recess 166. This allows the inner catheter portion 169, including the taper 162, to pass through an aperture in a lymphatic structure and then further allow the outer sheath 167 to longitudinally slide in a distal direction over the inner catheter portion 169 until its distal end contacts the wall of the lymphatic structure, thereby securing the distal tip of the catheter 116 to the wall of the lymphatic structure. Optionally, a proximal end of the catheter 116 may include a locking mechanism that can selectively prevent the inner catheter portion 169 and the sheath 167 from moving relative to each other once engaged with the lymphatic structure. If the length of inner catheter portion 169 from the taper 162 to the distal tip 168 is longer than a physician desires or is too long to accommodate a given patient's anatomy, the distal end of the inner catheter portion may be cut to the desired length to accommodate a wide variety of anatomical situations. However, the taper 162 and flange 164 of the inner catheter portion can also be longitudinally movable as previously described.

The inner catheter portion 169 may include the taper 162 or alternate shapes such as a flange 164, which have an outer diameter that is less than or equal to the diameter of the outer sheath 167 to allow retraction therein. Optionally, the tip 168 of the inner catheter portion 169 can be tapered or conical in shape (as with any of the catheter tips described in this specification). The inner catheter portion 169 and outer sheath 167 may be longitudinally slidable relative to each other or can be threaded with each other to maintain a desired distance between each other.

Alternatively, the taper 162 on the inner catheter portion 169 may contain magnetic material that is attractive to magnetic material in the outer sheath 167. If the length of catheter from the taper to the distal end is longer than a physician desires or is too long to accommodate a given patient's anatomy, the distal end of the catheter portion 169 may be cut to the desired length to accommodate a wide variety of anatomical situations.

As seen in FIGS. 21A, 21B, and 21C, a catheter 116 may also be configured to have an oblong or oval outer cross-sectional profile at its distal end to facilitate navigating valves in the lymphatic system. For example, distal region 170B may have an oval or oblong cross-sectional shape (FIG. 21B) while the remaining proximal portion of the catheter 116 has a generally circular cross-sectional shape (FIG. 21C). Alternately, the entire catheter may have an oval cross-sectional shape. The catheter 116 may be rotated such that the major axis (widest part) of the catheter is aligned with the widest opening of a valve or opening prior to advancing past the area. Various lymphatic structures in the body contain valves to prevent retrograde flow and promote antegrade flow, and therefore, the oblong cross-section profile may help navigate through these valves when the catheter 116 is properly rotated and aligned. This alignment may help reduce the risk of trauma or damage to the valve.

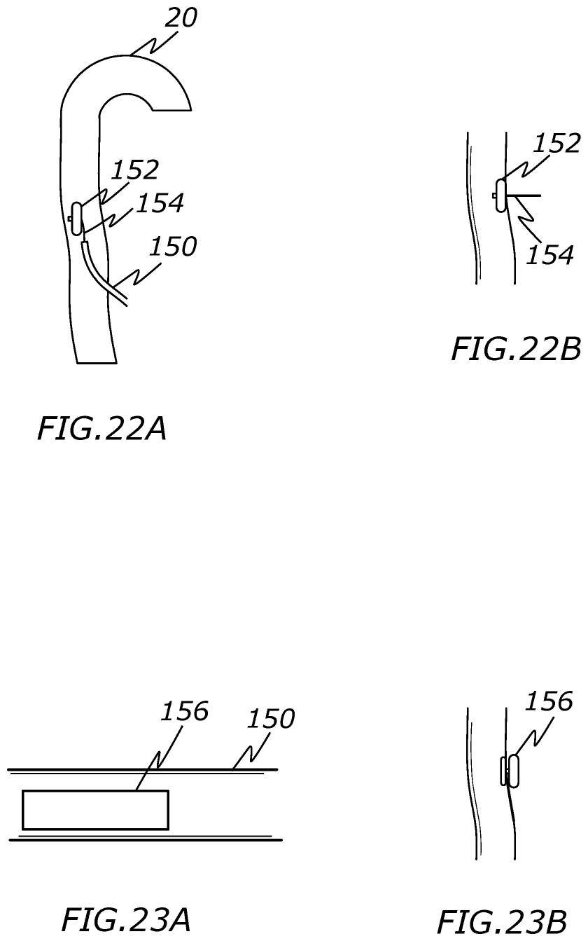

Following access and drainage of fluid from a lymphatic vessel, the drainage catheter 116 may be removed which leaves a hole in the lymphatic structure that can be closed with a closure device. FIGS. 22A and 22B illustrate one embodiment of a closure device comprised of a plug 152 with an attached suture 154. The plug 152 can be advanced through the drainage catheter 116 or a separate delivery catheter 150 can be used for delivery, as seen in FIG. 22A. When the catheter 150 is withdrawn from the lymphatic structure, the plug is pulled against the wall of the lymphatic structure by the suture 154 which is then tied to the surrounding anatomy by the physician to maintain its position. The plug 152 may be comprised of a textile, metallic, bioresorbable, or polymer material and have a structure that can compress and optionally self-expand to form a disc shape.

FIGS. 23A and 23B illustrate another embodiment of a closure device comprised of a plug 156 that can be deployed across the hole to directly engage the wall of the lymphatic vessel. The plug 156 may be designed to form a collapsed, substantially cylindrical shape within a delivery catheter 150 or drainage catheter 116. When the distal end of the catheter is positioned near or within the hole through the wall, the plug 156 is advanced at least partially out of the catheter so that it can expand to a disc shape sized to block the hole. The plug 156 may include a recess formed around the circumference of the plug 156, forming two outer edges that have a larger diameter than an internal region to create the shape of a dumbbell. These proximal and distal flanges preferably have a larger diameter than the hole in the lymphatic structure and are positioned so that the distal flange is located within the lymphatic structure and the proximal flange is located outside the lymphatic structure, as seen in FIG. 23B. This positioning helps to maintain the position of the plug across the hole. Additionally or alternatively, the plug 156 is at least partially deployed within the lymphatic vessel and then pulled through the hole until the plug 156 is aligned with the lymphatic vessel in the recess. The plug may contain an attached suture as shown in the prior embodiment of FIGS. 22A, 22B to aid in pulling the plug 156 into place. Additionally, the diameter of the distal flange may be similar to or different than the diameter of the proximal flange; for example, the distal flange diameter may be greater than the proximal flange diameter to ensure that the plug 156 is not pulled out of the lymphatic vessel during placement. Additional retention mechanisms may be further used with the plug 156, such as adhesive or sutures. The plug 156 may be comprised of a self-expanding material, such as nitinol, that is able to be collapsed and expand on its own (e.g., a nitinol wire, mesh, or other framework). The plug 156 may be further covered in a material that is impermeable to lymphatic fluid, such as polyurethane or silicone, to prevent fluid leaking into other areas of the body. The proximal-to-distal length of the internal region of the plug 156 may be designed to be substantially similar to or smaller than the width of the wall of the lymphatic structure to prevent dislodgement and leaking around the plug 156. The diameter of the internal region of the plug 156 may be designed to be substantially similar to or larger than the size of the hole in the lymphatic structure to prevent leaking around the plug 156.

As discussed in various embodiments below, the lymphatic system of a patient can also be accessed by transvenous access techniques via the circulatory system (e.g. artery or vein in the body). It should be understood that any methods and embodiments described in this specification can be used according to these techniques unless specifically indicated otherwise.

In one method, venous access in the arm or leg may be obtained per standard practice and an access device (e.g., guidewire, catheter) may be introduced into the body. The access device may be advanced toward the confluence of the subclavian vein and the internal jugular vein where the thoracic duct terminates. Upon reaching the desired location, contrast may be injected, and the anatomy visualized under X-ray or fluoroscopy to identify the terminal valve of the thoracic duct. Once visualized, the access device may be advanced through the terminal valve and into the thoracic duct and to a desired location with a lymphatic structure.

In another method, venous access in the arm or leg may be obtained per standard practice and an access device (e.g., guidewire, catheter) may be introduced into the body. The access device may be advanced toward the confluence of the subclavian vein and the internal jugular vein where the thoracic duct terminates. Separately, a needle may be advanced through the skin and into a lymphatic structure such as the thoracic duct, or cisterna chyli. A separate guidewire may be advanced through the needle and into the lymphatic structure in an antegrade manner until the guidewire passes the terminal valve of the thoracic duct and into the venous system. The access device in the vein (e.g., a snare catheter) may then snare the guidewire from the thoracic duct and then be advanced along the guidewire into the thoracic duct. The guidewire may be removed, and the access device may then be advanced to a desired location within a lymphatic structure.

In another method, venous access in the arm or leg may be obtained per standard practice and an access device (e.g., guidewire, catheter) may be introduced into the body. The access device may be advanced toward the confluence of the subclavian vein and the internal jugular vein where the thoracic duct terminates. Separately, contrast may be injected into a lymphatic structure (e.g., cisterna chyli, thoracic duct, lymph node, interstitial space, etc.) and allowed to follow the normal flow pattern of the lymph into the thoracic duct. Under X-ray or fluoroscopic visualization, the terminal portion of the thoracic duct may then be identified, and the access device may be advanced into the thoracic duct and to a desired location with a lymphatic structure.

In another method, venous access in the arm or leg may be obtained per standard practice and an access device (e.g., guidewire, catheter) may be introduced into the body. The access device may be advanced toward the confluence of the subclavian vein and the internal jugular vein where the thoracic duct terminates. Upon reaching the desired location, an ultrasound probe may be placed on the skin to identify the terminal valve of the thoracic duct. Once identified, the access device may be advanced through the terminal valve and into the thoracic duct and to a desired location with a lymphatic structure.

In another method, venous access in the arm or leg may be obtained per standard practice and an access device (e.g., guidewire, catheter) may be introduced into the body. The access device may be advanced toward the confluence of the subclavian vein and the internal jugular vein where the thoracic duct terminates. Upon reaching the desired location, a sensor apparatus on the catheter may measure one or more physical or physiologic signals including but not limited to fluid color, fluid leukocyte concentration, fluid salinity, fluid pH, fluid protein content, fluid white blood cell content, fluid velocity, presence of pulsatile flow, pressure, vessel diameter, vessel wall thickness, and vessel wall motion to identify the location of the terminal valve of the thoracic duct. Once identified, the access device may be advanced through the terminal valve of the thoracic duct and into the thoracic duct and to a desired location with a lymphatic structure.

In another method, venous access in the arm or leg may be obtained per standard practice and an intravascular imaging catheter and guidewire may be introduced into the body. The intravascular ultrasound may be used to identify the terminal valve of the thoracic duct from within the vein. Once it is identified, the guidewire may be advanced through the terminal valve of the thoracic duct and into the thoracic duct. A catheter may then be advanced over the guidewire and into the thoracic duct and to a desired location with a lymphatic structure.

In another method, ultrasound may be used to identify the terminal valve of the thoracic duct. Once it is identified, an access device such as a needle may be advanced through the skin, through the wall of a blood vessel, and oriented toward the terminal valve of the thoracic duct and into the thoracic duct. A guidewire may then be advanced through the needle, through the terminal valve of the thoracic duct, and into the thoracic duct and the needle may be retracted. A catheter may then be advanced over the guidewire and into the thoracic duct and to a desired location with a lymphatic structure.

In another method, venous access in the arm or leg may be obtained per standard practice and an access device (e.g., guidewire) may be introduced into the body. The access device may be advanced through the Azygos vein and toward the cisterna chyli. The cisterna chyli may be identified by any method known to one skilled in the art including but not limited to intranodal lymphangiography, MRI, CT, and Ultrasound. Once the cisterna chyli is identified and confirmed to be in close proximity to the Azygos vein, the access device may cross the lumen of the Azygos vein and into the lymphatic structure to provide access to the lymphatic fluid for removal. The crossing may be performed by a needle, sharp guidewire, Radio-Frequency guidewire, or other means known to one skilled in the art. Once the access device completes the crossing, a catheter may be advanced over the access device and into the cisterna chyli and to the desired lymphatic structure.

Alternatively, access to a lymphatic structure may be obtained via the esophagus by navigating to a position near a lymphatic structure and crossing into the desired lymphatic structure. Alternatively, access to a lymphatic structure may be obtained via the lungs by navigating through the bronchi to a position near a lymphatic structure and crossing into the desired lymphatic structure. Alternatively, access to a lymphatic structure may be obtained via the aorta by navigating to a position near a lymphatic structure and crossing into the desired lymphatic structure.

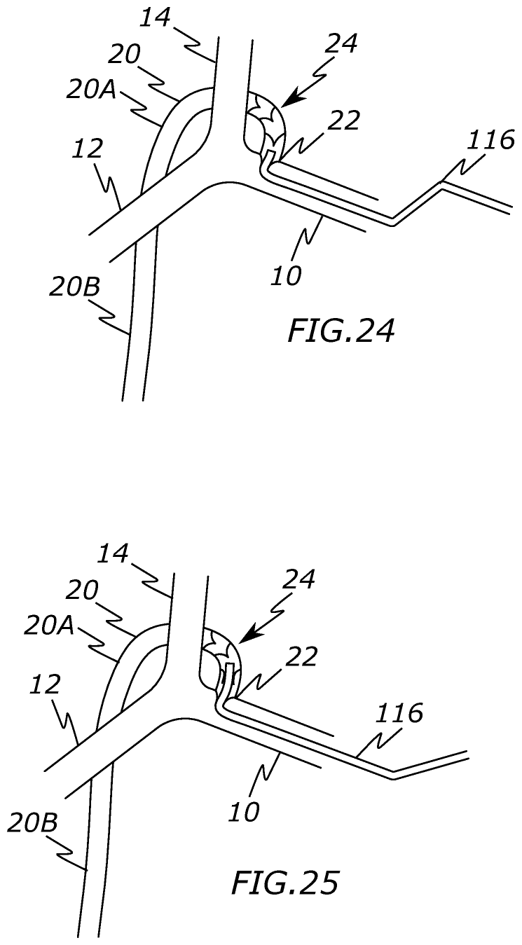

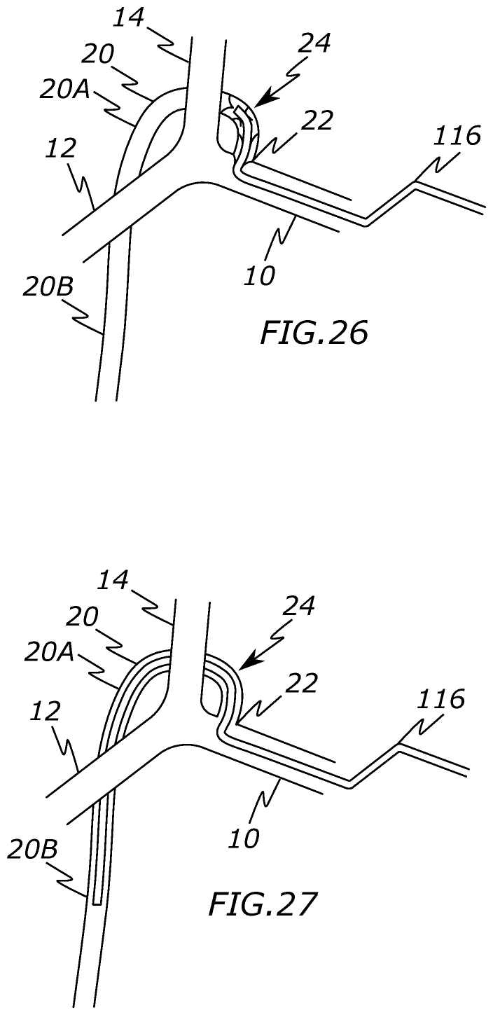

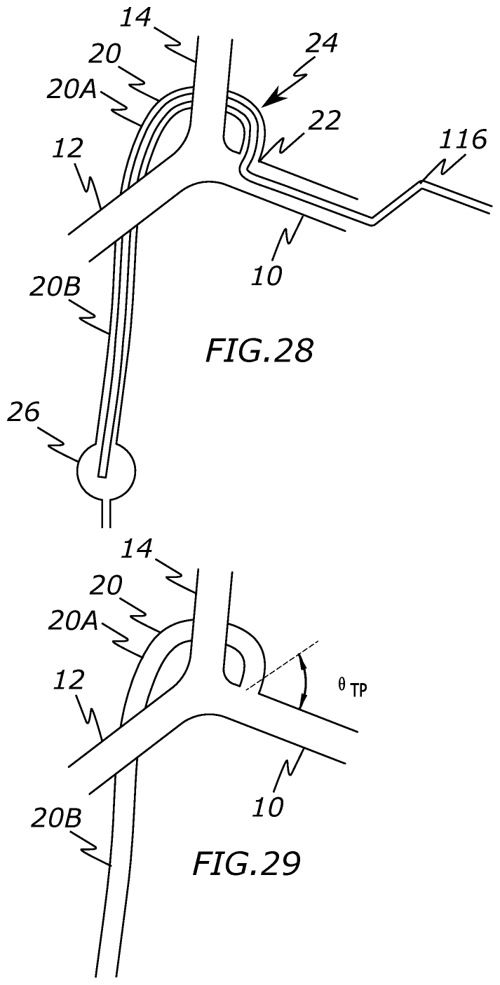

Once the distal end of the access device is in the thoracic duct, the distal end of the catheter may reside near the termination of the thoracic duct in the venous system. For example, FIG. 24 illustrates an access device (e.g., catheter 116) with its distal end positioned through only the terminal valve 22 (i.e., between the first and second valves). In another example, FIG. 25 illustrates catheter 116 with its distal end positioned through the terminal valve 22 and the next closest valve (i.e., between the second and third valves). In another example, FIG. 26 illustrates catheter 116 with its distal end positioned through the third valve (i.e., between the third and fourth valve). In another example, FIG. 27 illustrates catheter 116 with its distal end positioned beyond the distal four valves 24 in the thoracic portion 20B of the thoracic duct 20 (or alternately in the cervical portion 20A). In yet another example, FIG. 28 illustrates catheter 116 with its distal end positioned in the cisterna chyli 26.

Depending on the intended target position of the distal end of the access device (e.g., catheter 116), the access device may have several different lengths. The catheter may have a length within a range inclusive of about 5 centimeters (cm) to about 150 centimeters (cm) (e.g. 5 cm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 100 cm, 105 cm, 110 cm, 115 cm, 120 cm, 125 cm, 130 cm, 135 cm, 140 cm, 145 cm, or 150 cm).

For simplicity, the thoracic duct will be used as an example of a lymphatic structure in the following descriptions of specific embodiments; however, any lymphatic structure may be used without deviating from the present invention.

As seen in FIG. 29, a plane across the opening of the thoracic duct 20 and the axis of the subclavian vein 10 will generally form an angle, theta, relative to each other. When performing an approach to the thoracic duct 20 from the subclavian vein 10, it can be helpful to utilize devices with a similar curve, theta, to achieve access into the thoracic duct 20.

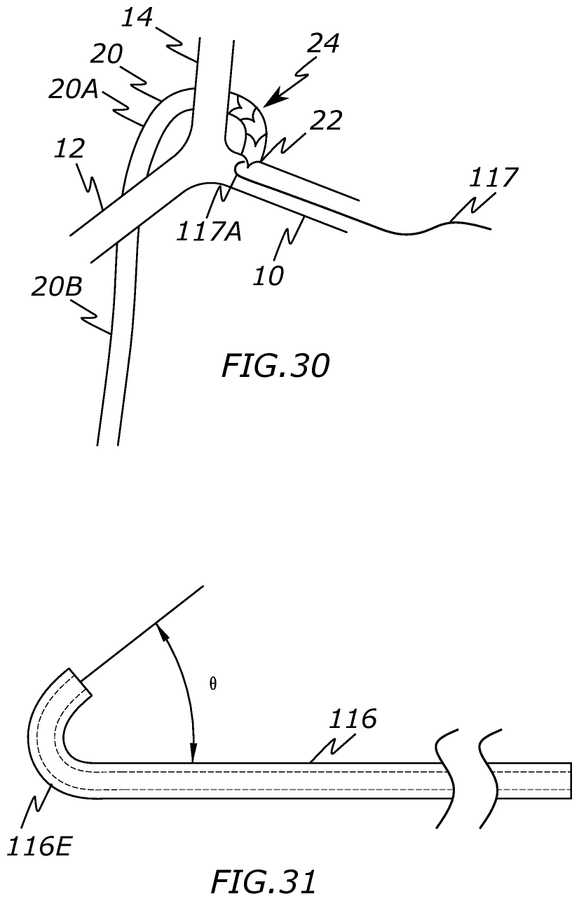

For example, FIG. 30 illustrates a guidewire 117 having a distal tip portion 117A that has a similar curve angle, theta, as between the thoracic duct 20 and the subclavian vein 10 which thereby allows easier access int the terminal valve 22 of the thoracic duct 20 (e.g., similar to the catheter 116 shown in FIG. 31, discussed below). A guide catheter 116 or similar device can then be advanced over the guidewire into the thoracic duct 20.

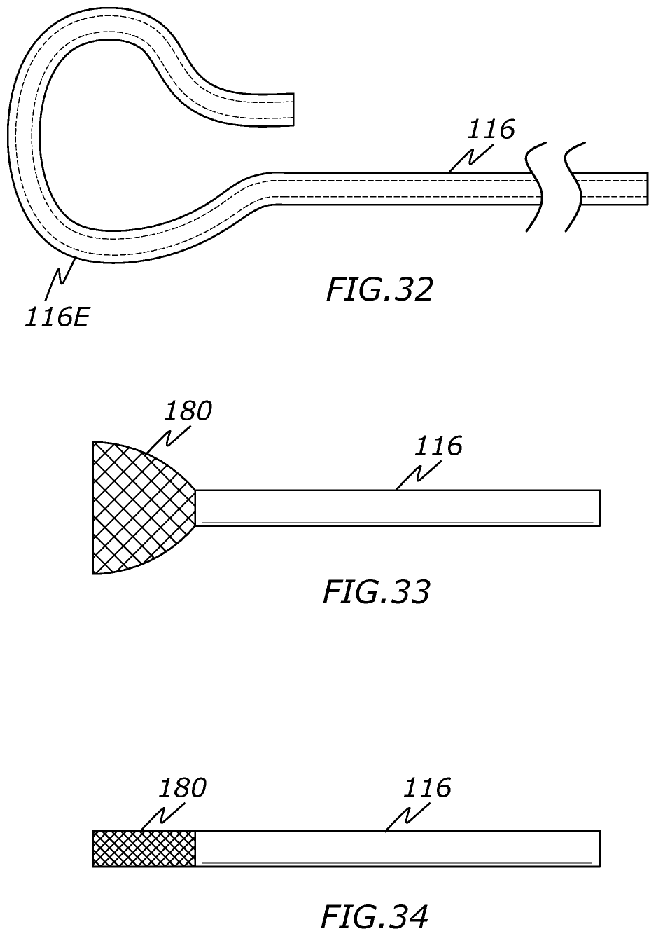

FIG. 31 illustrates another embodiment in which a catheter 116 (or similar access device) includes a distal end 116E with one or more simple or compound curves to align the tip of the catheter 116 more easily with the terminal valve 22 of the thoracic duct 20. The distal end 116E can be positioned within the subclavian vein 10 such that its distal opening is pointing at the terminal valve 117A. A flexible guidewire may be advanced through the lumen of the shaped catheter 116 to cross the terminal valve 22 and access the thoracic duct 20. Additionally, an access device such as a guidewire or catheter may contain echogenic or fluorogenic features such as marker bands or contain materials such as steel or barium to enhance visualization during navigation of the device or to confirm correct positioning following the placement of the device. In the example of FIG. 31, the distal end 116E has a simple curve that curves only in a single direction, however, more complex curves with multiple different degrees and angles are also possible, such as the example of FIG. 32 in which the distal end 116E curves in a first direction and then in an opposite direction.

In order to maintain a desired position of the catheter 116 within the desired lymphatic structure, one or more mechanism of fixation may be employed. In one embodiment shown in FIGS. 33 and 34, the catheter 116 includes an expandable distal portion 180 to anchor in the thoracic duct 20. The expandable distal portion 180 may be comprised of a shape-set material such as a nitinol wire, mesh, or framework; a flexible mesh material such as spring steel that may be actuated by a pull-wire; or a deformable material such as stainless-steel mesh that may be expanded in situ by a balloon. The distal portion 180 may expand or cause to become expanded from a generally uniform tubular shape (FIG. 34) to a conical or radially enlarged shape (FIG. 33).

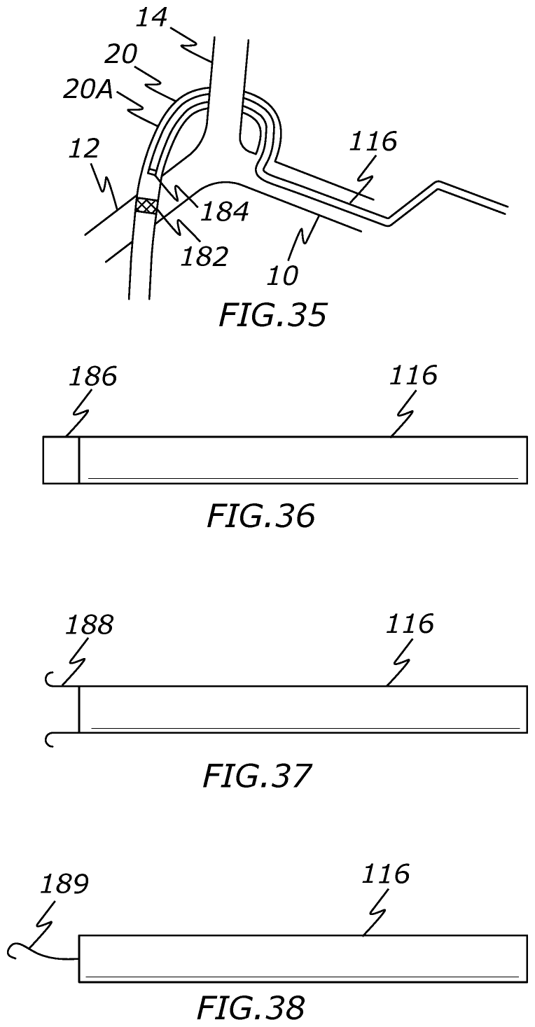

In another embodiment seen in FIG. 35, one or more attachment mechanisms 184 on the distal end of the catheter 116 are configured to connect to a stent 182 that has been previously placed in the lymphatic structure, such as the thoracic duct 20. In that respect, the distal end of the catheter 116 can be connected and anchored in place within the lymphatic structure. The attachment mechanism 184 may be configured to not only attach to the stent 182, but also disconnect from the stent 182 so that the catheter 116 can be removed.

In one example embodiment seen in FIG. 36, the attachment mechanism comprises one or more magnets 186. The magnets 186 can be disposed radially around the edge of the distal end of the catheter 116 and the stent 182 may also have a plurality of attached magnets configured to attract the magnets 186 (e.g., the magnets 186 may be configured and positioned to have opposing polarities so as to attract each other).

In another example embodiment seen in FIG. 37, the attachment mechanism comprises one or more hooks 188. For example, a plurality of the hooks can be configured to selectively radially expand (e.g., from a control wire extending to a proximal end of the catheter 116) and can curve radially outward from the circumference of the catheter 116 such that they can hook onto loops, mesh, or other features of the stent 182.

In another example embodiment seen in FIG. 38, the attachment mechanism comprises one or more wires 189. The wire 189 may be advanced through a central lumen of the catheter 116 and beyond the distal end of the catheter 116. The distal end of the wire 189 may be comprised of a hook shape to facilitate attaching to the stent 182 (e.g., through a loop of or opening in the stent 182). Once the wire 189 is attached to the stent 182, the catheter 116 may be advanced over the wire 189 and into a desired proximity to the stent 182. The wire 189 may then be secured to the catheter 116 to facilitate maintaining the catheter in the desired proximity to the stent 182.

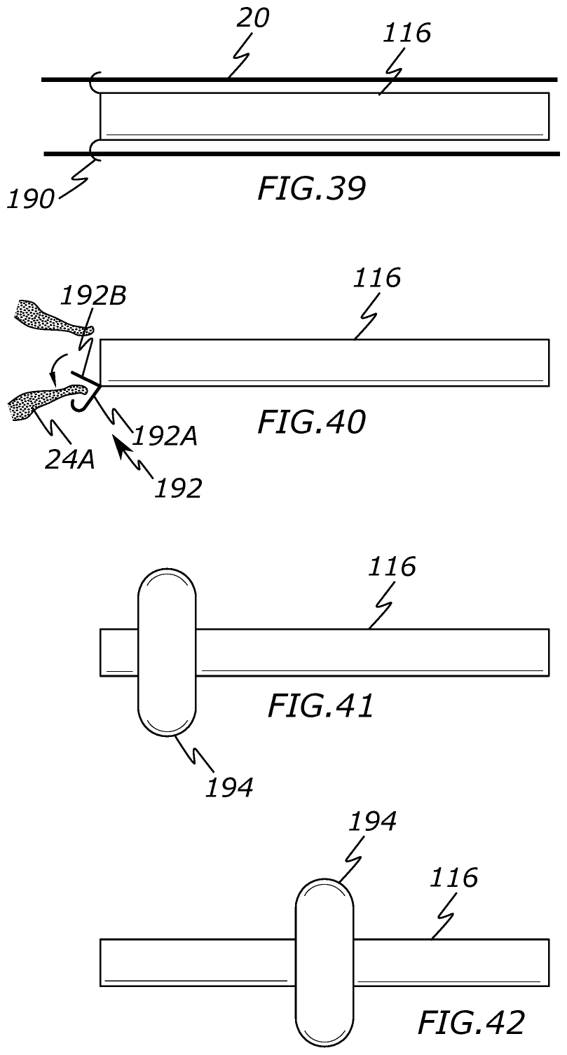

In another embodiment illustrated in FIG. 39, a catheter 116 may have hooks 190 that are sized and shaped to directly engage the walls of the thoracic duct 20. The hooks 190 may be comprised of a flexible material such as nitinol, steel, spring steel, stainless steel, or polymer. The hooks 190 may be attached to or integrated into the distal end of the catheter 116 with the hooks 190 having a shape biased away from the axis of the catheter 116 and toward the proximal end of the catheter 116 so as to prevent the catheter from moving toward the proximal end of the catheter 116 once the hooks 190 are engaged. The distal tips of the hooks 190 may extend beyond the outer diameter of the catheter 116 and define a diameter of a circle connecting the distal tip of each hook 190 (e.g., the tips of the hooks 190 may extend radially beyond the catheter's side surface by 0.5 mm, 1 mm, 2 mm, 3 mm, 4 mm, 6 mm, or 7 mm). This diameter may be equal to or greater than the outer diameter of the catheter 116 to allow the hooks 190 to engage the lumen of the thoracic duct. This diameter may be within a range inclusive of about 125% to 300% of the outer diameter of the catheter 116 (e.g. 125%, 150%, 175%, 200%, 225%, 250%, 275%, 300%) or within a range inclusive of about 2 mm to 15 mm (e.g., 2 mm, 3 mm, 4 mm, 5 mm, 6 mm, 7 mm, 8 mm, 9 mm, 10 mm, 11 mm, 12 mm, 13 mm, 14 mm, or 15 mm). Prior to insertion and advancement of the catheter 116, an outer sheath (not shown) may be advanced over the outer surface of the catheter 116 to straighten hooks 190 to prevent engagement during insertion of the catheter 116. Once the catheter 116 is in the desired location, the outer sheath may be retracted to allow the hooks to engage the thoracic duct 20. If the catheter 116 needs to be removed, an outer sheath may be advanced over the catheter 116 to straighten the hooks 190 and disengage from the thoracic duct 20. The catheter 116 and outer sheath may be removed together to avoid the hooks 190 from engage other tissue during removal.

In another embodiment illustrated in FIG. 40, the catheter 116 may grasp a valve leaflet 24A in the thoracic duct 20 via a grasping mechanism 192. For example, a hook 192A and an elongated lever member 192B extend distally from a distal end of the catheter 116. One or both of the lever 1926 and hook 192A can pivot or otherwise move relative to each other (e.g., controlled by control wires at a proximal end of the catheter 116) so that both components can selectively move towards and away from each other. When a leaflet 24A of the lymphatic system is located between the hook 192A and lever 192B, moving both components towards each other causes physical engagement to the valve leaflet 24A and thereby provides anchoring force.

In another embodiment, the catheter 116 may comprise a balloon 194 located close to the distal end of the catheter 116, as seen in FIG. 41. The balloon 194 may be inflated within the thoracic duct 20 and sized to be either 1) small enough to avoid occluding the thoracic duct 20 so as allow lymph fluid to flow around it but large enough to prevent migration of the balloon 194 or 2) substantially the same size as the thoracic duct 20. The diameter may be with a range inclusive of about 125% to 300% of the outer diameter of the catheter 116 (e.g., 125%, 150%, 175%, 200%, 225%, 250%, 275%, 300%) or within a range inclusive of about 2 mm to 15 mm (e.g., 2 mm, 3 mm, 4 mm, 5 mm, 6 mm, 7 mm, 8 mm, 9 mm, 10 mm, 11 mm, 12 mm, 13 mm, 14 mm, or 15 mm).

In another embodiment seen in FIG. 42, the catheter 116 balloon 194 is located in a middle section of the catheter 116 to maintain its location within the venous system and, by virtue of the mechanical properties of the catheter 116, maintain its position within the lymphatic system. For example, the balloon 194 may be anchored in the left subclavian vein 10 and have an expanded diameter of about 15 mm, 17.5 mm, 20 cmm, 22.5 cm or 25 mm. Optionally, the balloon 194 may be shaped to allow perfusion past it, including features such as axial perfusion passages.

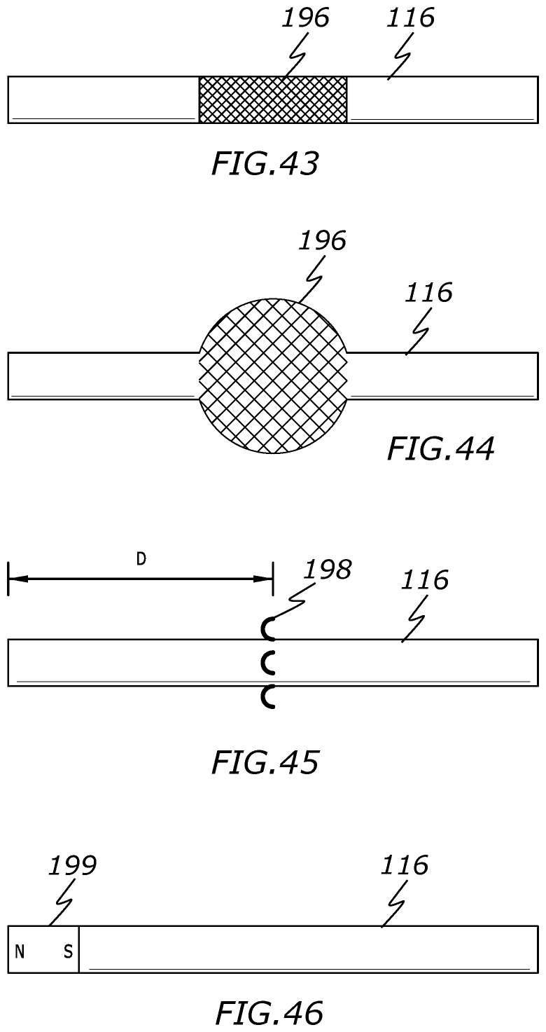

In an alternate embodiment, the balloon 194 can be replaced with a selectively expandable mesh 196 having a size sufficient to anchor within a body vessel of the patient but with a mesh pore size sufficient to allow fluid flow therethrough. FIG. 43 illustrates the mesh 196 in its unexpanded state having a uniform diameter and FIG. 44 illustrates the mesh 196 in its radially expanded state. The mesh can be controlled via an elongated pull wire that pulls or pushes an end of the mesh 196 in a longitudinal direction, or the mesh can be self-expanded to its expanded state. The mesh 196 can be located at the distal end of the catheter 116 for anchoring in a lymphatic structure or at the middle section of the catheter 116 to anchor in a blood vessel. Hence, similar example expanded sizes are possible as with previously discussed balloons 194.

In another embodiment illustrated in FIG. 45, the catheter 116 may have hooks 198 in a middle section of the catheter 116 that are sized and shaped to directly engage the lumen of a vessel through which the catheter 116 is introduced. The distance D from the hooks 198 to the distal end of the catheter 116 may be sufficient to maintain the position of the distal end of the catheter 116 in the desired lymphatic or blood vessel. For example, the hooks may be spaced greater than 40 cm, 35 cm, 20 cm, 9 cm, or less than 5 cm from the distal end of the catheter 116. The hooks 198 may be retractable by attachment to one or more control wires that are accessible at the proximal end of the catheter 116.

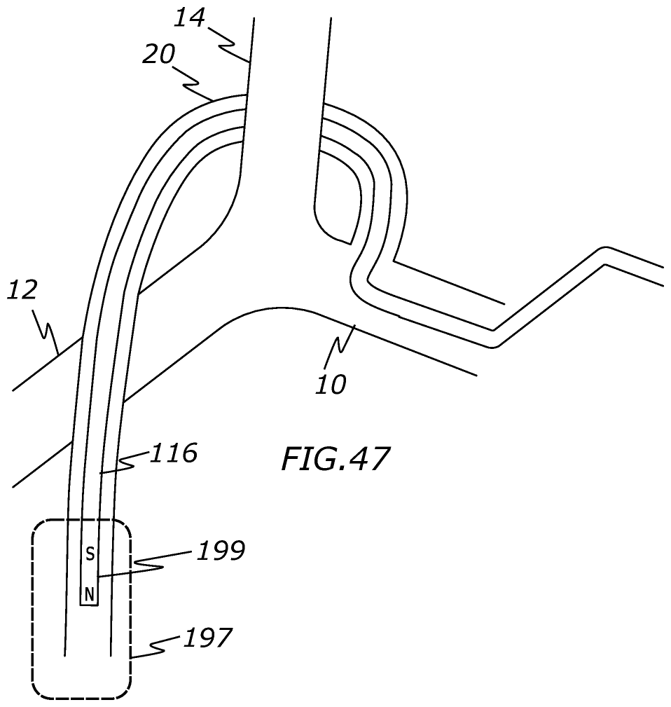

In another embodiment seen in FIGS. 46 and 47, the catheter 116 may be comprised of a magnetic tip 199 that is magnetically attracted to a device 197 positioned externally of the patient. For example, the external device may also include a relatively strong permanent magnet or electromagnet which allows the physician to position the device over or adjacent to the desired anchor location of the magnetic tip 199 within the patient. Additionally, the external device can be used to help guide the magnetic tip 199 prior to anchoring and drainage.

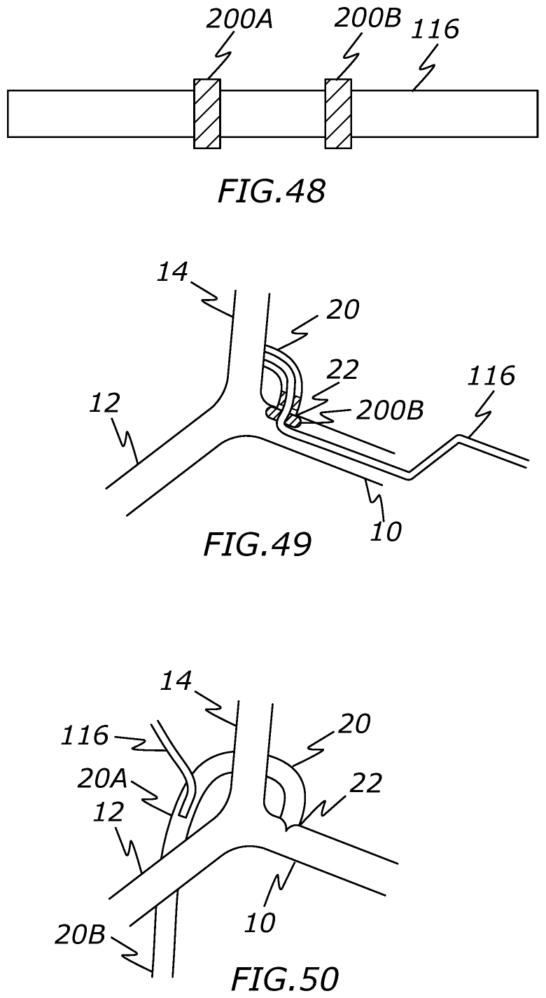

In another embodiment shown in FIGS. 48 and 49, a catheter 116 includes a distal expandable member 200A and a proximal expandable member 200B that may be expanded on either side of the terminal valve 22 of the thoracic duct 20 or another valve of the thoracic duct 20 to maintain its position. The expandable members 200A, 200B may be composed of balloons or an expandable mesh similar to those previously described in this specification. The expandable members 200A, 200B can be simultaneously expanded or one can be expanded prior to the other's expansion. It may also be desirable for the distal expandable member 200A to expand to a smaller diameter than the proximal expandable member 200B. For example, the distal expandable member 200A may expand to about 5 mm, 7.5 mm, 10 mm, 12.5 mm, or 15 mm and the proximal expandable member 200B may expand to about 15 mm, 17.5 mm, 20 cm, 22.5 mm, or 25 cm.

As discussed in various embodiments below, the lymphatic system of a patient can be accessed and drained with a catheter or similar access device having a proximal end exterior to the lymphatic system of a patient and at least one lumen exposed or in communication with lymphatic fluid in a lymphatic structure. For simplicity, the thoracic duct 20 will be used as an example of a lymphatic structure; however, any lymphatic structure may be used without deviating from the present invention. It may be appreciated that the embodiments below, while described or drawn using a particular method of accessing and placing a drainage device, may be used in conjunction with a variety of different methods of accessing and placing a drainage device in a lymphatic structure, especially as described elsewhere in this specification. These embodiments are intended as illustrative examples and do not serve to limit the scope of the present invention.

In one embodiment seen in FIG. 50, a drainage device (e.g., a drainage catheter 116) may be introduced directly into the lymphatic system without first entering the patient's venous system as described elsewhere in this application. This allows a drainage lumen opening at its distal end to be in communication with the interior of the lymphatic system.

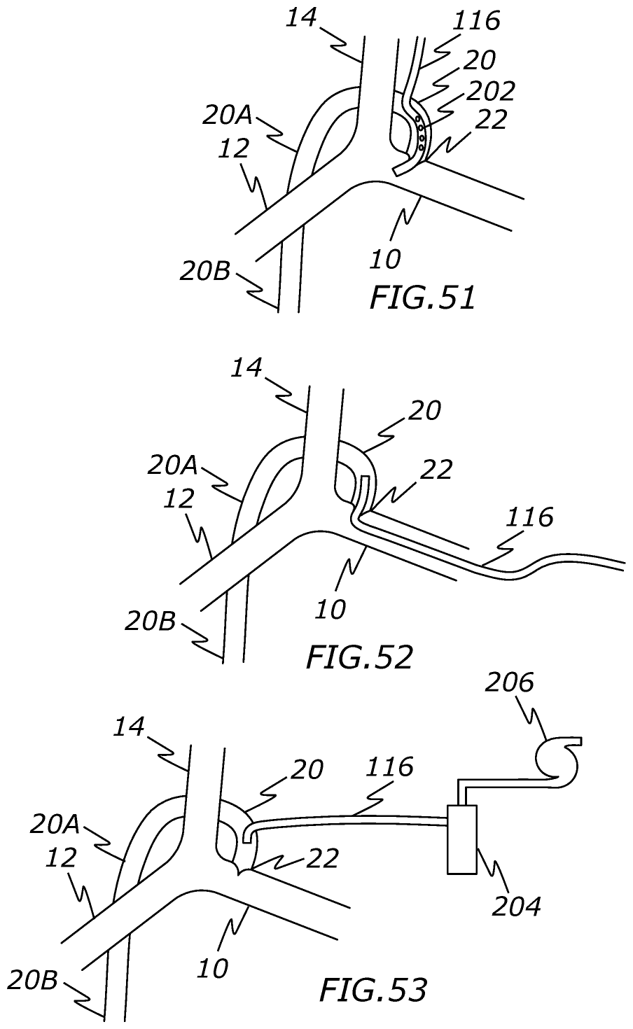

In another embodiment seen in FIG. 51, the drainage catheter 116 may be introduced directly into the lymphatic system with its distal end positioned in the venous system and one or more drainage features in the lymphatic system. For example, the catheter 116 can be positioned into the thoracic duct 20 and its distal end can be further positioned through the terminal valve 22 into the left subclavian vein 10. The distal portion of the catheter 116 may include a plurality of drainage apertures 202 opening to a drainage lumen within the catheter 116. The drainage apertures 202 can be positioned such that they are located only within the thoracic duct 20.

In another embodiment seen in FIG. 52, the drainage device may be introduced in a transvenous manner with the distal end of the drainage lumen in the lymphatic system. For example, the drainage catheter 116 can be advanced through the left subclavian vein 10, through the terminal valve 22, and into the thoracic duct 20 as described elsewhere in this application.

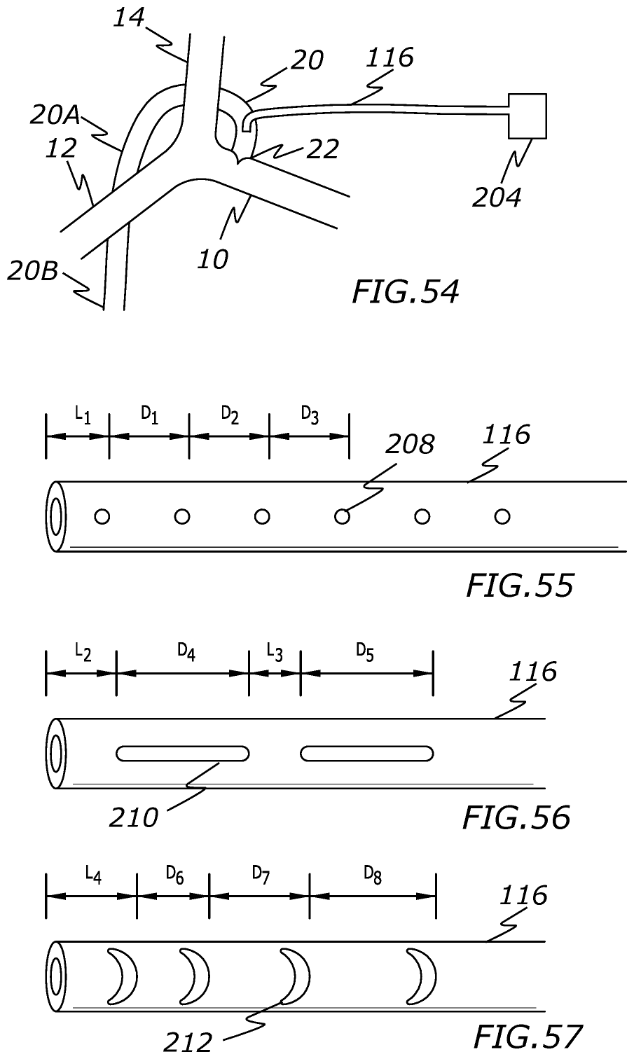

In order to change the flow rate of lymphatic fluid from the lymphatic vessel, the height of the proximal tip of the drainage catheter 116 may be raised or lowered relative to the position of the patient to change the native driving pressure from the patient. For example, a reservoir 204 connected to the catheter 116 can be raised or lowered relative to the patient, as seen in FIG. 54.

Alternatively, a suction source may be attached to the drainage catheter to reduce the back pressure in the system and increase the total driving pressure of the flow to increase the flow rate. As further seen in FIG. 53, a suction source (e.g. suction pump 206) may be applied in a continuous or intermittent manner. The suction source 206 may be connected to the drainage device via a reservoir 204 to collect the fluid or may directly pump the fluid from the drainage catheter 116.

In another embodiment, the drainage device may be comprised of a catheter with one or more lumens and one or more drainage features which may be comprised of one or more drainage holes, slots, profiles, or features. The features may be oriented in a substantially axial orientation, radial orientation, a combination of both, or neither. For example, FIG. 55 illustrates a plurality of circular apertures 208 that extend longitudinally and are spaced apart from each other (distance D) and from the end of the catheter 16 (length L) wherein the spacing between circular apertures 208 (distances D1, D2, D3) can be equal or different. Additionally, the circular apertures 208 can be arranged in a linear pattern as shown in FIG. 55 or could be arranged in a spiral pattern to maximize the likelihood of an aperture being open to fluid if the catheter 116 were to be in contact with a body lumen. In another example, FIG. 56 illustrates a plurality of elongated, slot-like apertures 210 that are longitudinally aligned at the distal end of the catheter 116. In another example, FIG. 57 illustrates a plurality of curved or half-moon shaped apertures 212 (depending on the angle these may also appear as semicircular shapes or ellipses) that could be created by a skiving operation and that are longitudinally aligned at the distal end of the catheter 116.

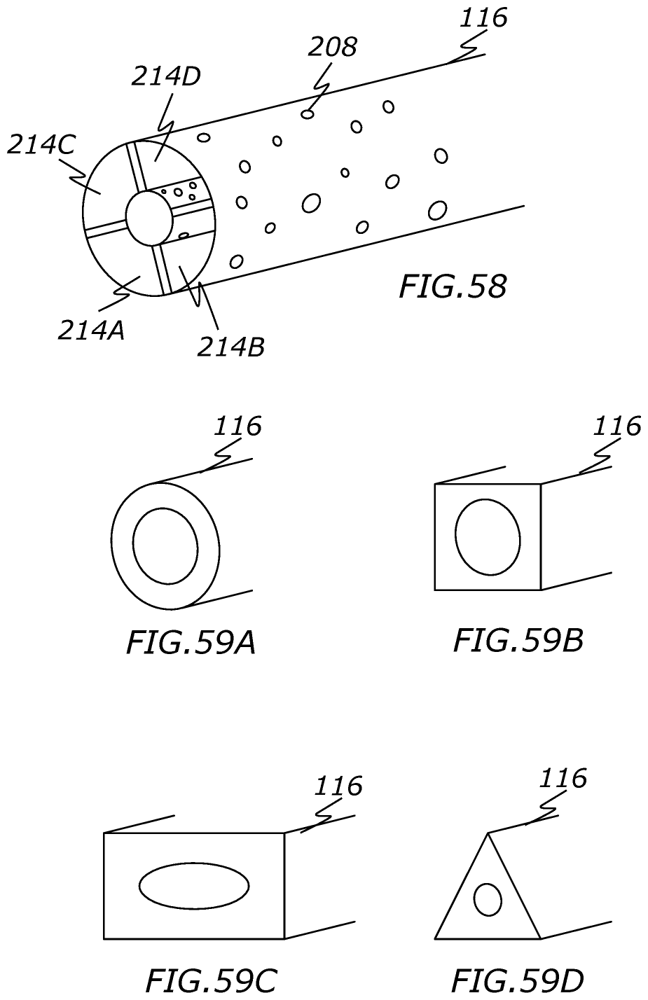

The features may intersect a single lumen or may intersect a plurality of lumens in the catheter 116. For example, FIG. 58 illustrates a plurality of round apertures 208 that open to one of four different lumens 214A-214D within the catheter 116. These extra lumens can provide redundancy in case one feature were to become occluded by a clot or against a vessel wall.

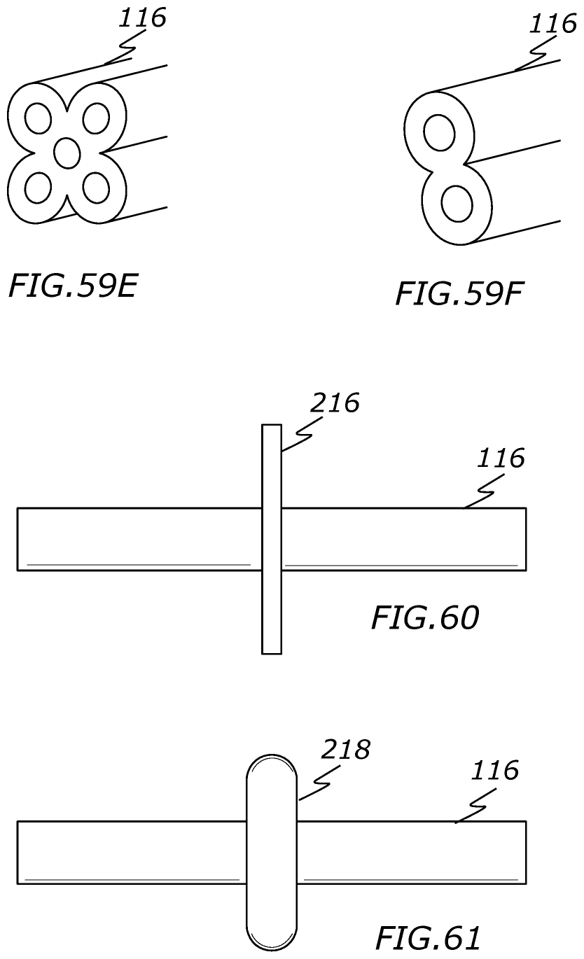

Additionally, the outer cross-sectional profile or shape of the catheter 116 may be circular (FIG. 59A), square (FIG. 59B), rectangular (FIG. 59C), triangular (FIG. 59D), two adjacent figure-8 shapes (FIG. 59E), a single figure-8 shape (FIG. 59F), or any other shape known to one skilled in the art.

In another embodiment, the drainage device may have a sealing member placed proximal to the drainage features to prevent blood from flowing retrograde from the venous system into the thoracic duct 20. Generally, the sealing member expands to a diameter that is larger than the opening of the thoracic duct 20 or has a diameter sufficient to occlude an interior of the thoracic duct 20. The sealing member can further include material, seals, or other components to assist in creating a seal around the thoracic duct opening or against its interior.

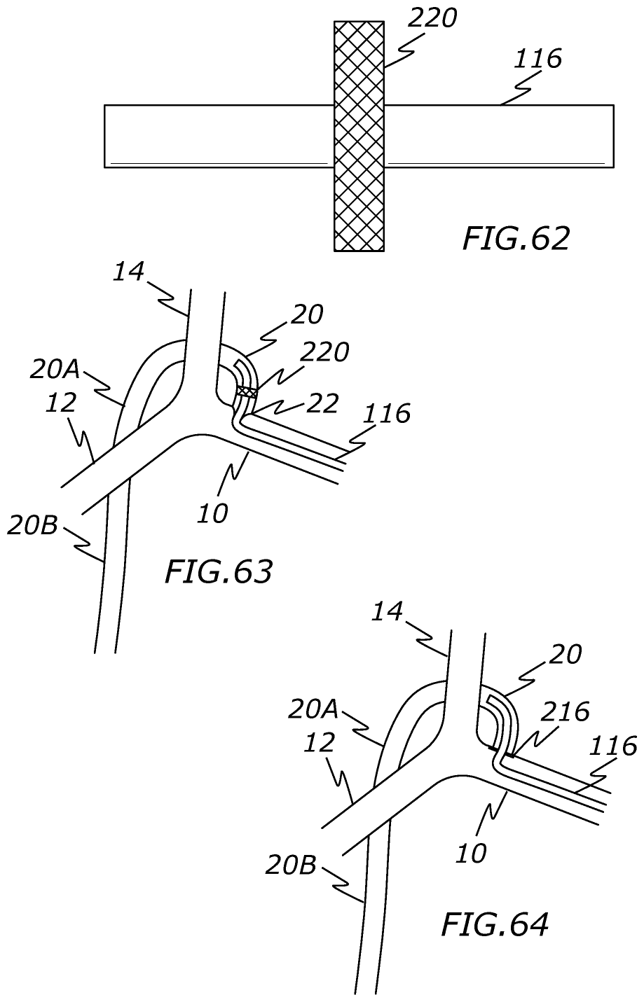

For example, FIG. 60 illustrate a flexible flange member 216 composed of a material that self-expands to a generally perpendicular profile relative to the axis of the catheter 116. The flange 216 may be composed of a polymer such as polyurethane, polyethylene, Teflon, or Pebax or a more flexible material such as silicone. In another example, FIG. 61 illustrates a balloon 218 that can be selectively inflated to help seal the thoracic duct 20. In yet another example, FIG. 62 illustrates an expandable mesh 220 that may either self-expand or be manually expanded (e.g., via a control wire). The expandable mesh 220 may have a covering that is impermeable to fluid (e.g., a polymer) to help block fluid past the mesh 220. As seen in FIG. 63, the sealing member can be expanded within the thoracic duct 20 (or other areas of the lymphatic system) or at the opening of the thoracic duct 20, with the left subclavian vein 10.

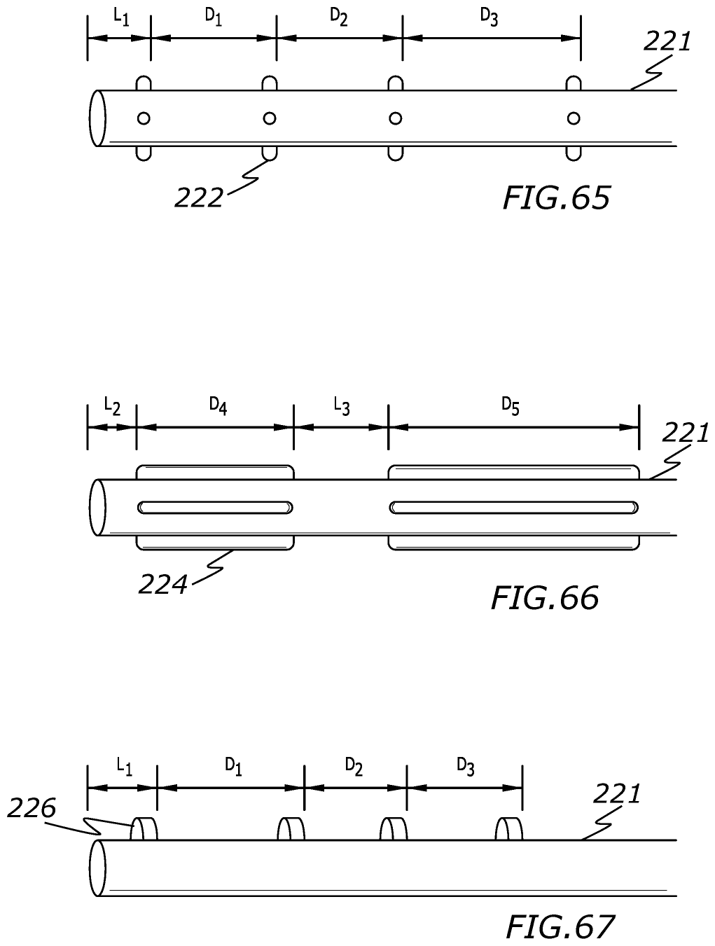

In order to maintain the flow of lymphatic fluid from the lymphatic vessel, one or more anti-clogging mechanism may be employed. For example, a cleaning stylet 221 may be used to clean a drainage lumen within a drainage catheter 116 by advancing and/or rotating the stylet 221 through the lumen and pushing any clogging materials back into the lymphatic vessel. The cleaning stylet may also unclog the one or more drainage features (e.g., apertures within the catheter wall) with radially-oriented features such as protrusions or fibers that may be advanced into the holes to dislodge any clogging. For example, FIG. 65 illustrates a stylet 221 with an elongated body and a plurality of flexible, rounded bumps or pegs 222. In another example seen in FIG. 66, the style 221 may include a plurality of raised, flexible, elongated shapes 224. In another example, FIG. 67 illustrates a plurality of raised, flexible shapes 226 that extend at least partially around a circumference of the catheter 116 (e.g., cylindrical or ellipse shape). In another example, a stylet 221 can include any combination of these shapes.

The cleaning stylet's protrusions may substantially match the size and shape of the draining features. The cleaning stylet's protrusions may be comprised of a flexible material to enable compression during advancement through the drainage catheter and expansion to dislodge clogging materials from the drainage features. The cleaning stylet 221 may be advanced axially within the drainage catheter and/or rotated to ensure the cleaning features access the entirety of the drainage lumen and drainage features. Alternatively, a high-pressure source of fluid may be connected to the drainage lumen and fluid may be forced into the lymphatic vessel to dislodge any clogging materials. Alternatively, a suction source may be connected to the drainage lumen and vacuum applied to the drainage catheter to dislodge any clogging materials. In another embodiment, the drainage lumen of the catheter 116 may be coated in a hydrophilic material that resists the attachment of any potential clogging materials.

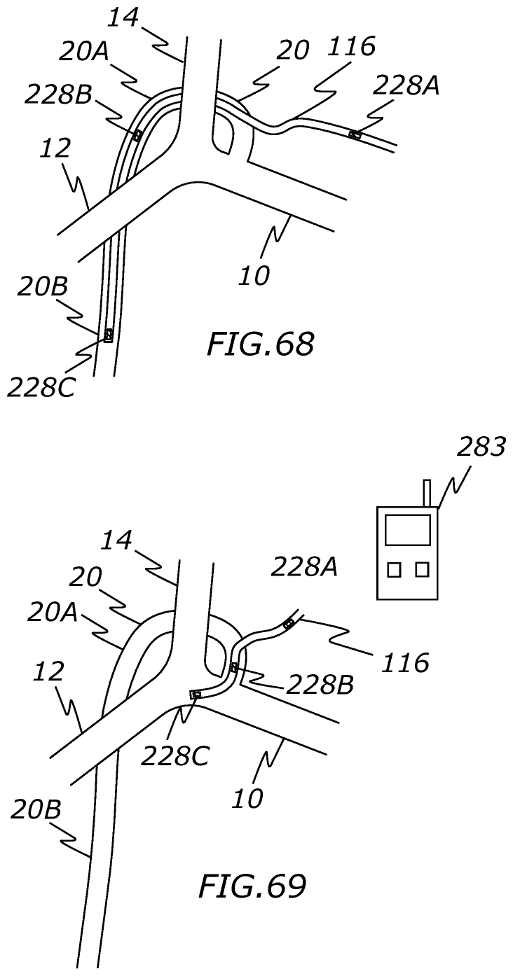

In another embodiment, the drainage device may have one or more sensors to detect various signals. The one or more sensors may be on the outer surface of the catheter 116 or within the internal lumen of the catheter 116. Additionally, the one or more sensors may be placed near the distal end of the catheter, near the middle of the catheter, or near the proximal end of the catheter. For example, FIG. 68 illustrates a catheter 116 with a proximal sensor 228A, a middle sensor 228B, and a distal sensor 228C. In this example, at least the distal sensor 228C and middle sensor 228B are located within the thoracic duct 20. In another example, FIG. 69 illustrates the distal sensor 228C being positioned within the left subclavian vein 10 while the middle sensor 228B is located within the thoracic duct 20. In that respect, the drainage device may be placed partially in the lymphatic system and partially in the venous system. In another example (not shown), the distal sensor 228C is located within the thoracic duct 20, the middle sensor 228B is located within the patient but external to the thoracic duct 20 (e.g., within the venous system or even outside the venous system but within the patient), and the proximal sensor 228A is located external to the patient.

The one or more sensors 228 may be spaced apart by 2.5 cm, 5 cm, 7.5 cm, 10 cm, 12.5 cm, 15 cm, 17.5 cm, 20 cm, 30 cm, 40 cm, 50 cm, or 75 cm from each other, for example. In the case of the proximal sensor 228A, the aforementioned distance may be between the proximal sensor 228A and the proximal end of the catheter. The distal sensor 228C may also be spaced apart from the middle sensor 228B by a different distance than the proximal sensor 228A and the middle sensor 228B.

In one embodiment, the one or more sensors 228 may relay their data to a controller 283. The controller may at least include a processor, memory, and software executable by the processor. The software may include algorithms that include 1) measuring, receiving, and storing sensor data, 2) actuating devices (e.g., valves in a drainage system), and/or 3) generating notifications to a user and/or medical staff locally or remotely. The controller 283 may be a dedicated control unit, functionality integrated into another device (e.g., an existing monitor), or a smartphone/tablet.

The one or more sensors may measure the one or more signals and allow the calculation of the difference between the measurements including but not limited to the pressure difference between the thoracic duct and an adjacent venous vessel such as the brachiocephalic vein, internal jugular vein, or subclavian vein.

In one embodiment, a drainage device may contain one or more sensors to determine if the drainage lumen within the drainage device is clogged and requires unclogging. The sensors may be positioned along the exterior surface of the catheter or along the interior surface of the drainage lumen. The one or more sensors may detect one or more of the following parameters including but not limited to the pressure in the drainage lumen at one or more locations, the pressure difference between a proximal sensor and a distal sensor, the flow rate through the drainage lumen, the presence of fluid or tissue at the drainage features, the location of tissue relative to the lymphatic vessel lumen. For example, if the pressure difference between a proximal sensor and a distal sensor is large, it may be indicative of a clog in the lumen and the unclogging procedure may be initiated. Alternatively, if the flow rate through the lumen decreased over time but the pressure difference between a proximal sensor and a distal sensor is small, it may be indicative of removing all of the fluid within the immediate region of the lymphatic vessel and unclogging is not required. Alternatively, if the pressure at a distal sensor suddenly drops, it may be indicative of the drainage device occluding against a wall of a lymphatic vessel and manipulation of the drainage device is required.

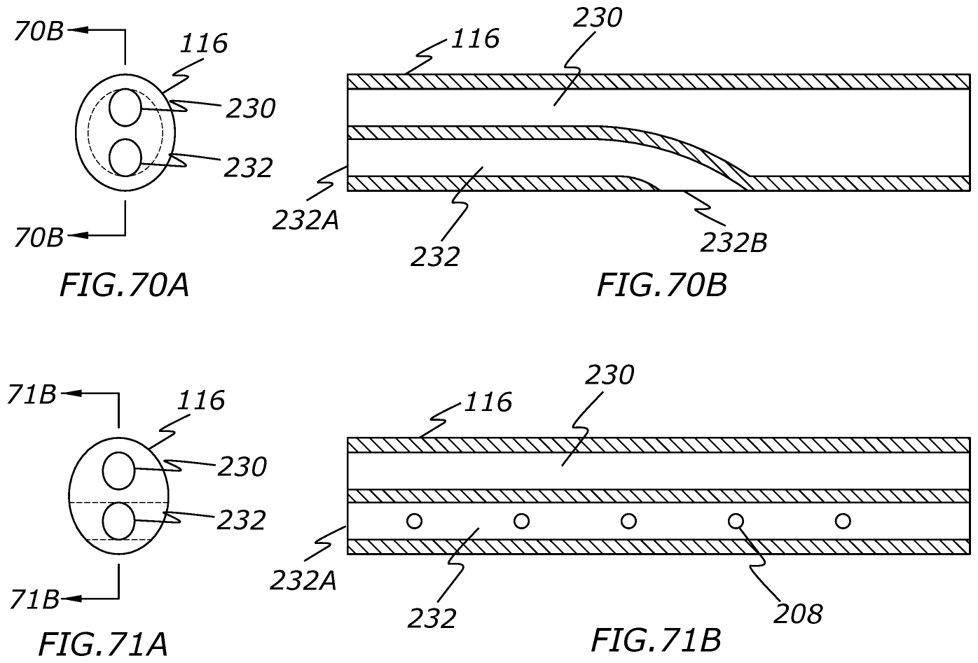

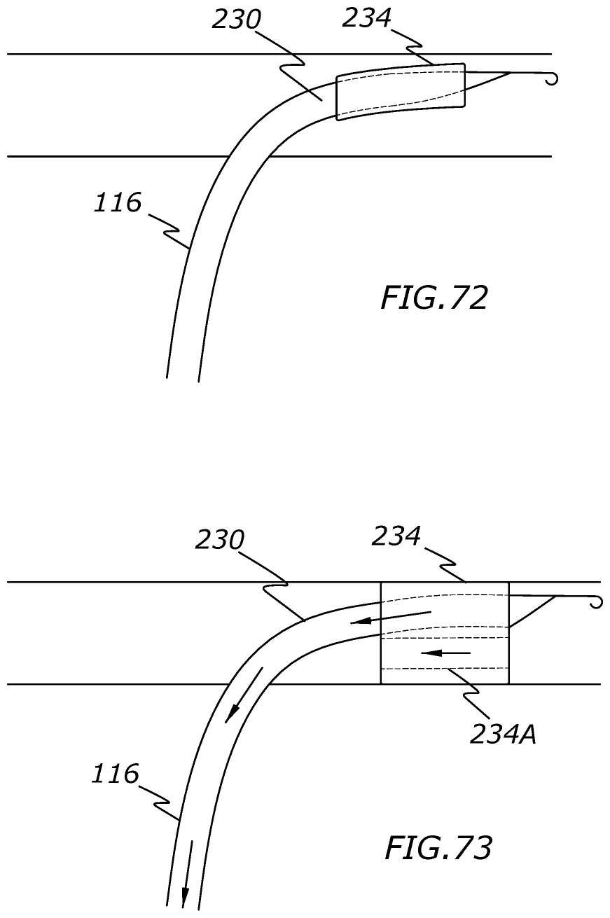

In another embodiment, the drainage catheter may contain bypass or perfusion features to allow flow of lymphatic fluid around or past the drainage catheter 116 during a procedure which may be advantageous to retain some constituents of the drainage fluid within the body including but not limited to white blood cells, leukocytes, protein, and electrolytes. For example, FIGS. 70A and 70B illustrate a catheter 116 having a drainage lumen 230 and bypass lumen 232 that has a distal opening 232A and a proximal opening 232B. The proximal opening 232B may be located at about 1 mm, 1.5 mm, 2 mm, 2.5 mm, 3 mm, 4 mm, or 5 mm away from the distal opening 232A. In another example, FIGS. 71A and 71B illustrate a similar bypass lumen having a distal opening 232A and a plurality of exit apertures 208 located longitudinally along the catheter 116 for the lymph fluid to escape and bypass the drainage lumen 230. In an alternative example seen in FIGS. 72 and 73, a distal end of the catheter 116 may include an inflatable perfusion balloon 234 that is configured to expand to a size sufficient to engage an internal lumen of the lymphatic system. The balloon 134 can be disposed around the circumference of the catheter 116 and include a pass-through lumen 234A extending between proximal and distal ends of the balloon 234 to allow lymphatic fluid flow past the balloon 234.

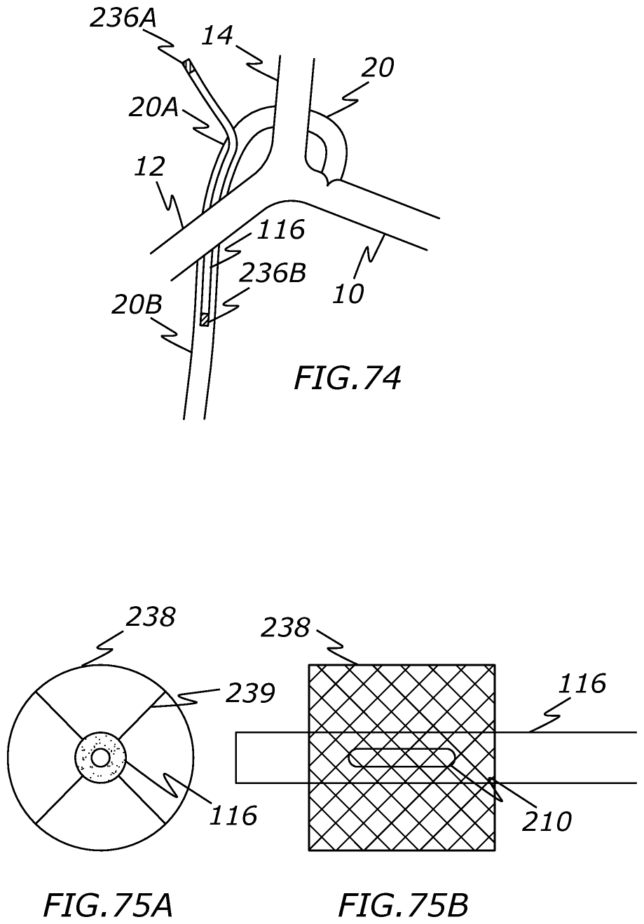

In another embodiment, the drainage device may incorporate a filter 236 along the fluid path to selectively remove one or more constituents from the lymphatic fluid. A filter 236A, 236B may only allow water or isotonic fluid to pass through, or may be configured to selectively block leukocytes from passing through the filter. The filter may be placed at the distal end of the catheter so only filtered fluid enters the lumen and/or the filter may be placed at the proximal end of the catheter for ease of replacement of the filter. The filter may be placed within the drainage lumen or attached in-line and exterior to the drainage lumen using a connector (e.g., a filter located within a Luer lock connector or similar device that is in-line with the drainage lumen). For example, FIG. 74 illustrates a first filter 236A located near a proximal end of the catheter 116 within its drainage lumen and a second filter 236B located near a distal end of the catheter 116 within its drainage lumen.

In another embodiment, the drainage device may contain a support mechanism to prevent collapse of the lymphatic vessel in the presence of suction. A support mechanism may expand and push the lymphatic vessel away from the drainage features of the drainage device to prevent the tissue from obstructing the drainage features.

In one example seen in FIGS. 75A and 75B, the support mechanism is framework comprising a tubular mesh portion 238 that is connected and supported relative to the catheter 116 by a plurality of struts 239. The support mechanism can be self-expanding or can be expanded by the physician via a control wire or similar mechanism. The support mechanism is picture as being positioned over an elongated slot 210, but any size and number of drainage apertures are possible.

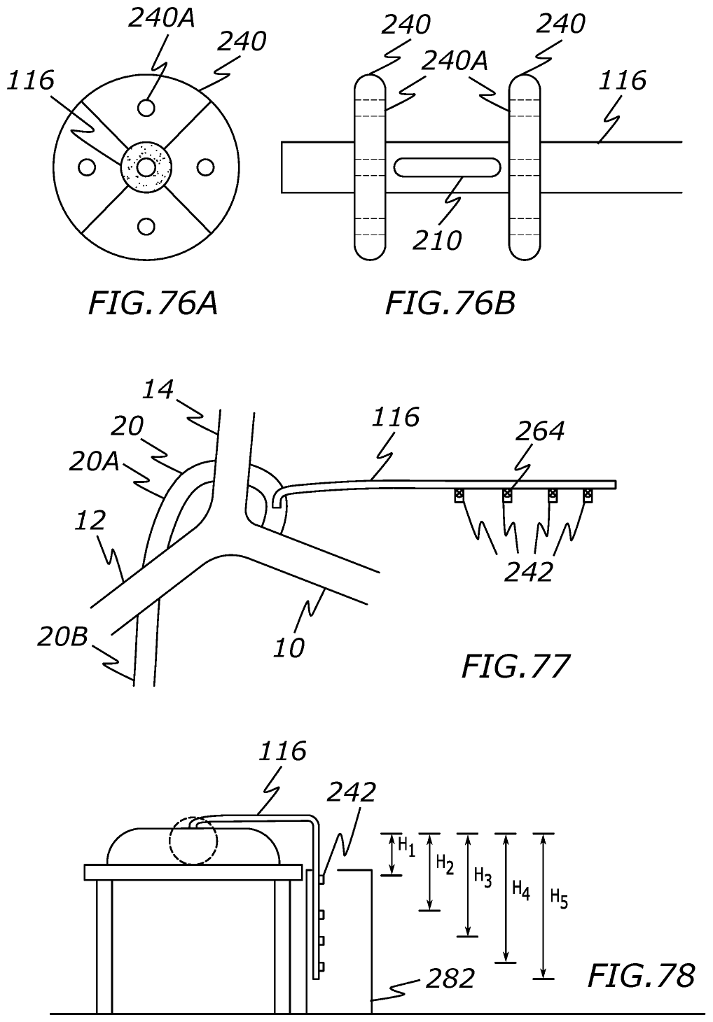

In another example seen in FIGS. 76A and 76B, the support mechanism includes one or more balloons 240 located near one or more drainage apertures 210. The balloon 240 preferably expands to a radius that allows the catheter 116 to be spaced apart from walls of the lymphatic system without causing stretching or damage. In the present example, a balloon 240 is located at the proximal and distal ends of one or more drainage apertures 210, however, depending on the number and location of the drainage apertures 210, 1, 2, 3, 4, 5, or more balloons 240 are possible. Since the balloons 240 tend to block fluid flow, one or more bypass or perfusion passages 240A may be included through each balloon 240. These passages 240A may extend between the proximal and distal ends of the balloon 240 and the passages 240A in a generally linear path to allow fluid to easily flow through. The passages 240A may be located radially inwards so that the balloon 240 completely surrounds the perimeter of passages 240A or may be located near the circumference so that the balloon 240 does not completely surround the passages 240A (e.g., notches or grooves in the outer surface of the balloon 240A).

In another embodiment, the drainage device may have a plurality of fluid outlets along its length to provide options for influencing the flow rate. The flow rate is impacted by both the pressure differential between the inlet and outlet and the resistance to flow. The plurality of fluid outlets may provide multiple locations from which to extract fluid. Due to the fact that there are pressure losses along the length of the lumen due to friction, the resistance to flow may be increased or decreased by selecting a fluid outlet with a longer distance or shorter distance from the fluid inlet, respectively. Additionally, the pressure differential between the fluid inlet and outlet is impacted by gravity and the vertical distance between the fluid inlet and outlet. Therefore, the fluid outlets may be oriented at different vertical distances relative to the fluid inlet and, therefore, adjust the pressure differential. For example, FIGS. 77 and 78 illustrates a drainage catheter 116 having a plurality of fluid outlets 242 positioned at different locations along the length of the catheter 116. The fluid outlets 242 may be comprised of valves 264 that may be opened or closed to achieve the desired pressure head (e.g. H1, H2, etc.). For example, these valves 264 may be stopcocks, an array of valves in a manifold for a manual actuation, or solenoids for an automatic actuation. The manifold may be comprised of a hollow block or tube with an array of valves and connections to control the direction of flow (e.g. from the inlet to the desired outlet of the manifold). As seen in FIG. 78, a fluid outlet 242 just below the fluid inlet (e.g. H1) may be chosen to reduce the pressure differential and a fluid outlet further below the fluid inlet (e.g. H4) may be chosen to increase the pressure differential. One or more of the plurality of fluid outlets 242 may be contained within a reservoir 282 to ensure fluid is captured regardless of which fluid outlet 242 is active at any given time. Alternatively, the proximal end of the drainage catheter may contain a tube around the outer surface of the fluid outlets to collect the drained fluid and deliver to a reservoir 282. The tube may be large enough that the draining fluid would not occlude the tube since occluding the tube with draining fluid may change or reduce the pressure differential within the drainage system and thereby prevent a desirable pressure gradient.

This specification is also directed to several different aspects of draining lymphatic fluid through a lumen of a drainage device having a proximal end terminating in a port or similar device that is selectively accessible from outside of a patient's body and at least one lumen exposed to lymphatic fluid in a lymphatic structure. For simplicity, the thoracic duct will be used as an example of a lymphatic structure; however, any lymphatic structure may be used without deviating from the present invention.

It should be understood that any methods and embodiments described elsewhere in this specification can be used with these access port-related embodiments and methods unless specifically indicated otherwise. More specifically, any of the methods or embodiments related to draining lymphatic fluid through a catheter lumen may be combined with any of the methods or embodiments including a port connected to the catheter for accessing the drainage lumen.

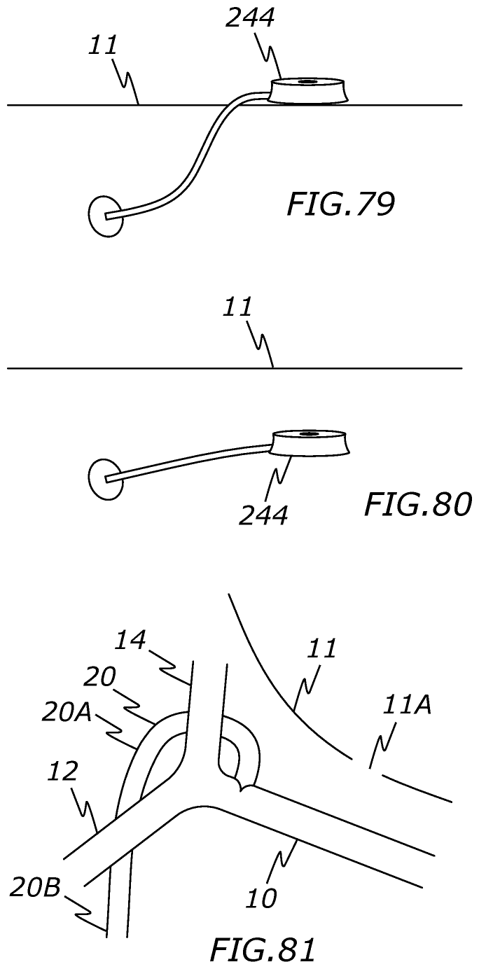

In one embodiment seen in FIG. 79, a catheter access port 244 may be placed on or above a patient's skin 11 with a connected catheter placed through the skin and into a lymphatic vessel. In another embodiment seen in FIG. 80, a catheter access port 244 may be placed underneath a patient's skin 11 with a connected catheter placed into a lymphatic vessel. It may be appreciated that the embodiments below, while described or drawn using a particular placement of the port on the skin or within the subcutaneous tissue, may be used in conjunction with either approach, and the embodiments are intended as illustrative examples and do not serve to limit the scope of the present invention.

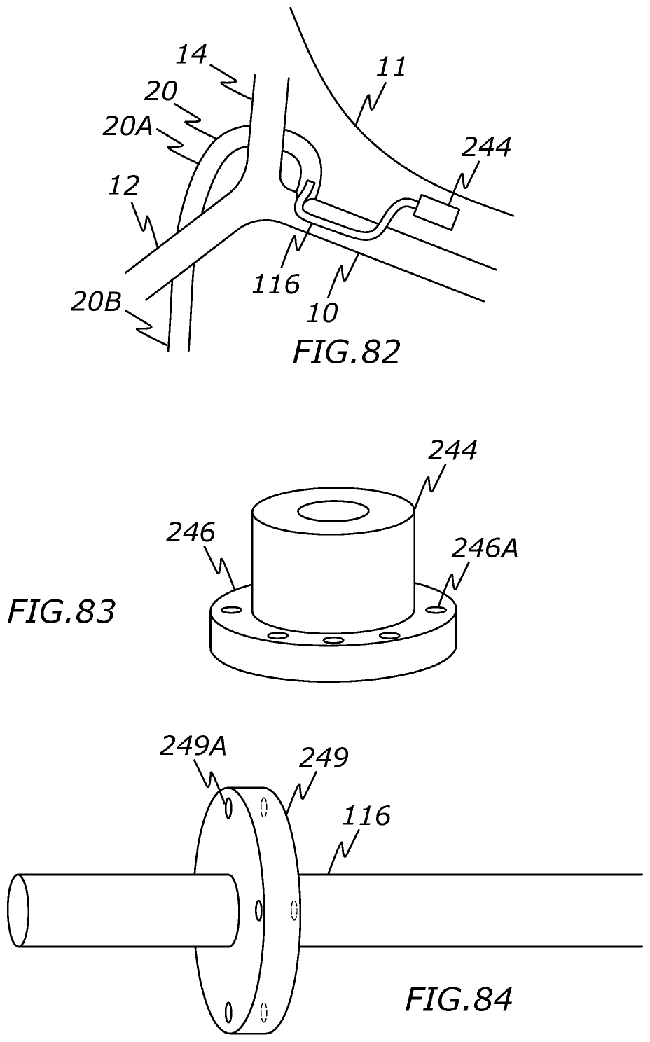

In order to implant a drainage device comprised of a catheter access port and catheter, a surgical cut-down procedure may be used to create an incision 11A within a patient's skin 11, as seen in FIG. 81. A distal end of the drainage catheter 116 may be placed within a lymphatic vessel either directly or through a vein or other body structure connected to the lymphatic system, as seen in the example of FIG. 82. A proximal end of the catheter 116 may be connected to a catheter access port 244 using a barb connector, adhesive connector, or other means known to one skilled in the art. The lymphatic vessel may be identified visualization means including direct visualization or manipulation, fluoroscopy or X-ray with contrast, lymphangiography, fluorescent visualization means such as indocyanine-green (ICG), or ultrasound.

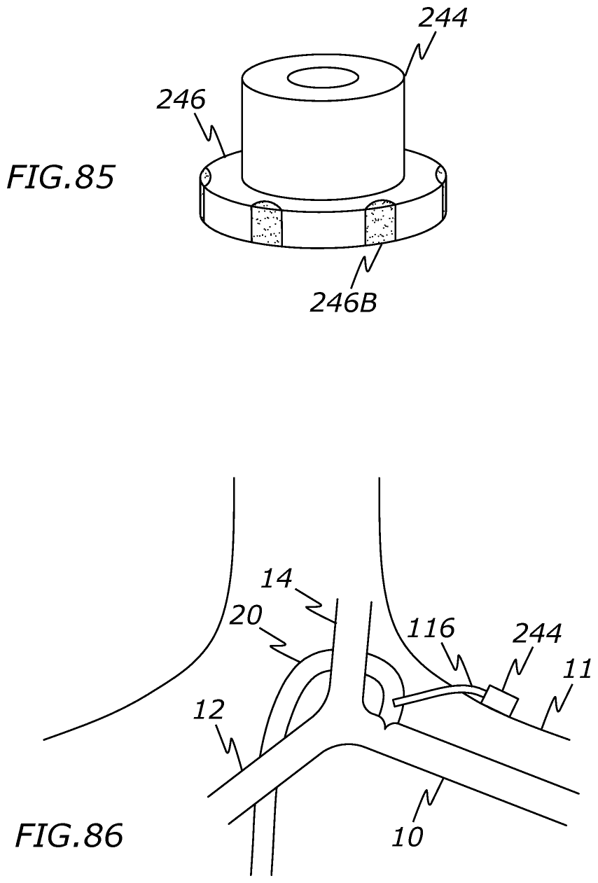

The port 244 may be implanted underneath the skin 11 in a desirable location such as the chest, abdomen, arm, neck, shoulder, back, or thigh. The port 244 and catheter 116 may contain one or more retention features to aid in maintaining a desired location and orientation within the body. For example, FIG. 83 illustrates a port 244 having a flange 246 on its lower end with one or more apertures 246A for connecting or allowing connection to other attachment mechanisms. Similarly, FIG. 84 illustrates a catheter 116 with one or more flanges 249 and having one or more apertures 249A. A suture or staple or clip may be passed through the one or more apertures 246A, 249A and adjacent body structures such as bone, connective tissues, or muscle to secure the port 244 or catheter 116 in place. Additionally, the port 246 may contain one or more features to enhance tissue ingrowth such as porous sections or rough sections 246B on its outer surface (e.g., on areas of the outer flange 246, as seen in FIG. 85). Additionally, the catheter 116 may be attached to the subcutaneous tissue or lymphatic vessel with any attachment means described elsewhere in this application.

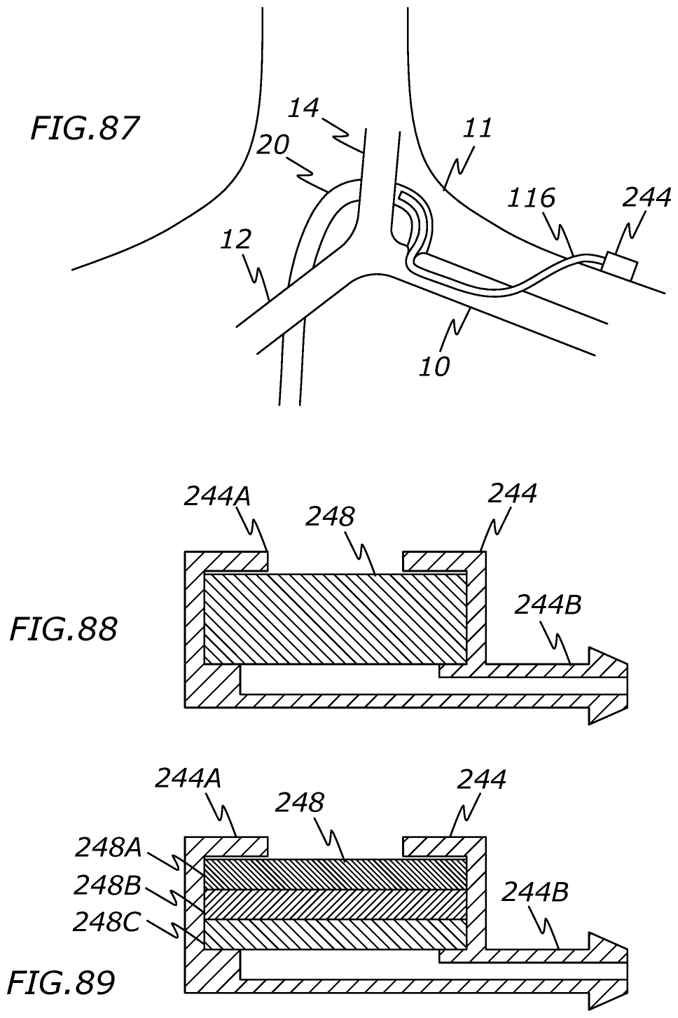

In another embodiment of implanting a drainage device comprised of a port and catheter, a surgical cut-down procedure may be used to expose a lymphatic vessel. A distal end of the catheter 116 may be placed within a lymphatic structure either directly (FIG. 86) or through a vein (FIG. 87) or other body structure connected to the lymphatic system. A proximal end of the catheter 116 may be connected to a catheter access port 244 outside of the body using a barb connector, adhesive connector, or other means known to one skilled in the art. The port 244 may be placed above the skin 11 in a desirable location such as the chest, abdomen, arm, neck, shoulder, back, or thigh. The port 244 and catheter may contain one or more retention features to aid in maintaining a desired location and orientation. In one embodiment, a port 244 may contain a flange with one or more apertures similar to those previously discussed. In another embodiment, the catheter may contain one or more flanges with one or more apertures similar to those previously discussed. A suture or staple or clip may be passed through one or more holes and adjacent body structures such as bone, connective tissues, or muscle to secure the port or catheter in place. Additionally, the port may contain an adhesive to attach to the skin.

Additionally, a catheter access port 244 may be comprised of a sealing assembly. In one embodiment, the sealing assembly may be comprised of a silicone material. In FIG. 88, the sealing assembly comprises a unitary silicone portion 248 that otherwise blocks the passage of fluid between the catheter inlet 244B and the drainage outlet 244A. This allows a tool such as a needle to penetrate and extend past the silicone portion 248 to communicate with the catheter inlet 244B. In another embodiment, multiple silicone layers 248A, 248B, 248C may be positioned near or directly against each other, as seen in FIG. 89. In one example, each of these layers may be composed of different materials or at least possess different properties (such as durometer or tear strength). In this regard, if one layer fails, the other layers act as a back-up. This can be particularly helpful with some needle designs that tend to core material in a seal, causing it to fail. Hence, a stack all of layers may be more robust and resistant to leakage and failure, especially through multiple uses.

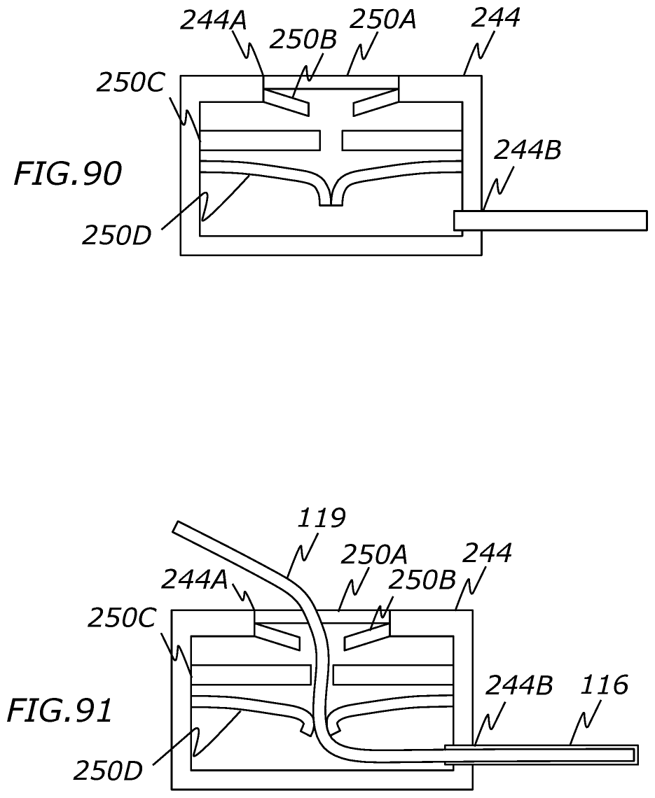

In one embodiment shown in FIGS. 90 and 91, the sealing assembly of the port 244 is configured to seal around either a single catheter 116 that is only temporarily and repeatedly placed within a patient or a second drainage catheter 119 that enters and connects to a first, previously implanted drainage catheter 116. In one example, the port 244 may include a needle puncture layer 250A, a needle-guiding layer 250B, a disc seal layer 250C, and a valve seal layer 250D. These layers may have different properties and functionality. For example, the needle puncture layer 250A may be a thick silicone layer as previously discussed for sealing portion 248. A needle-guiding layer 250B may be a more rigid/stiff disc/funnel shape that directs a needle 110 toward the open space underneath the sealing assembly. If the needle 110 enters at a very shallow angle, this layer 250B helps direct it to the right location. A disc seal layer 250C may be a disc with an aperture through it to seal around a catheter 116. The aperture cut out of the middle of the layer 250C may help the catheter 116 to pass through without stretching the material too much and causing a rip (i.e., similar to an annulus). The valve seal layer 250D may be a relatively less stiff layer that is configured as a one-way valve to prevent fluid from leaking out through the other layers if they were to be compromised due to repeated accesses. This layer 250D may be soft enough that anything placed through the rest of the layers would pass through this layer as well.

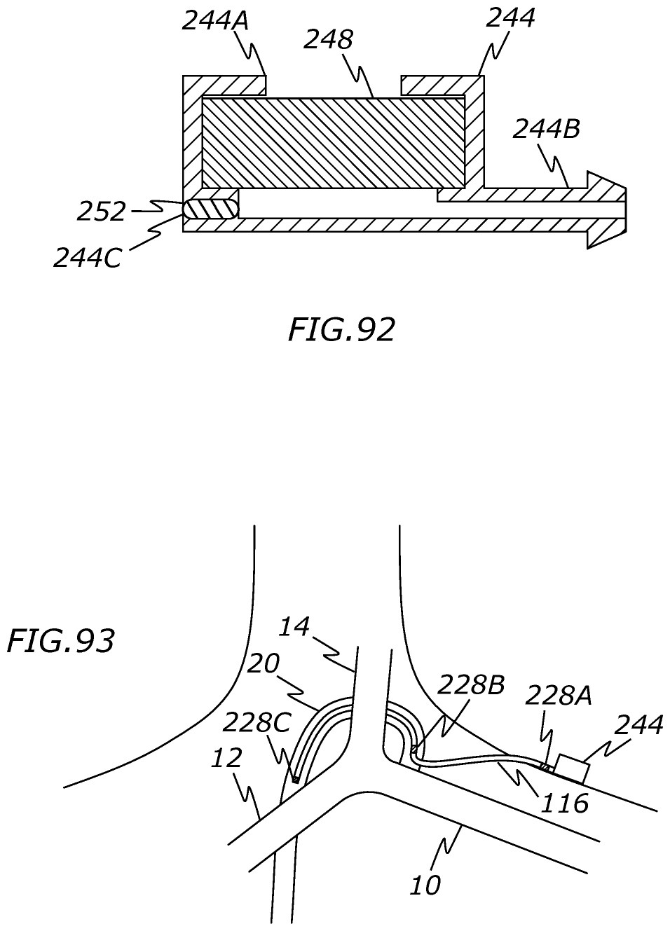

In another embodiment, a catheter access port 244 may be comprised of two or more openings in the housing, each being blocked by a sealing mechanism. For example, FIG. 92 is generally similar to the embodiment of FIG. 88 but includes a second outlet opening 244C that is closed by a second sealing portion 252. The second outlet opening 244C can be configured to allow access by an access device such as a needle. In that respect, it may be beneficial to orient the second outlet opening 244C in a direction along the width of port 244 to allow for more room for the needle to pass into the port 244. The second outlet opening 244C may also be aligned to form a substantially linear path or trajectory with the inlet 244B to facilitate passage of an access device such as a guidewire or catheter 116, 119. The second sealing portion can be composed of materials similar to those previously described in prior embodiments (e.g., silicone) and can be formed of only a single layer or multiple layers. Additionally, the second sealing portion may have different characteristics or properties than the first sealing portion 248, such as a greater tear resistance to accommodate a larger access device such as a catheter.

In one embodiment seen in FIG. 93, a catheter 116 extending from the catheter access port 244 may include one or more filters 228A-228C configured to only allow the desired fluid constituents to be removed, including but not limited to water, salt, and isotonic fluid. The one or more filters 228A-228C may be placed at the proximal, middle, and/or distal portions of the drainage catheter 116. The one or more filters 228A-228C may be located within the lumen of the drainage catheter or adjacent to the lumen such as at the distal end of the drainage catheter 116.

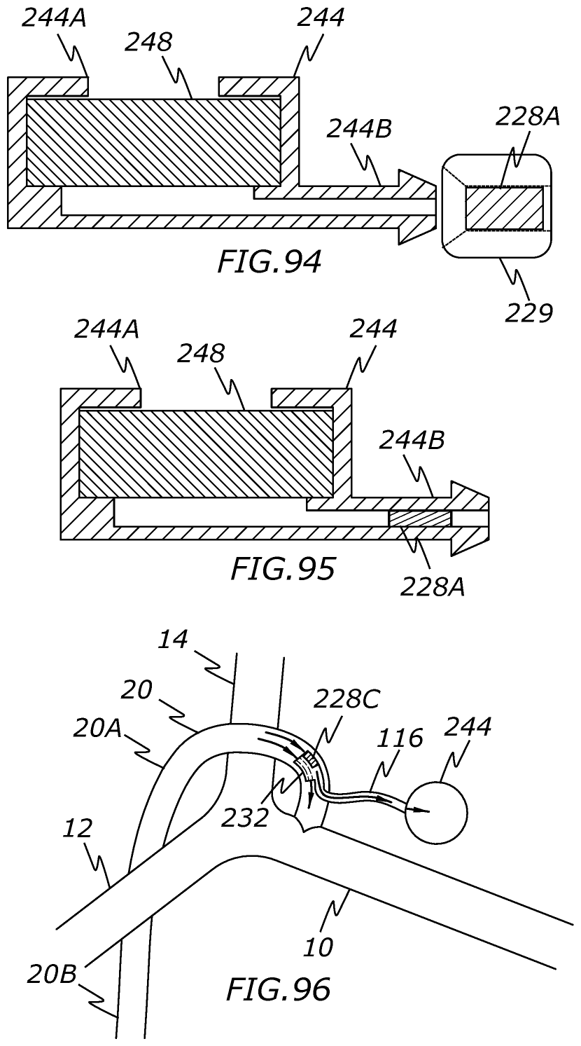

A proximal filter 228A may be comprised of a separate connector housing 229 placed in series with the inlet portion 244B and the drainage catheter 116, as seen in FIG. 94. Any connection mechanism can be used between the housing 229, inlet portion 244B, and catheter 116, such as magnetic connectors, quick-disconnect connectors, Tuohy-Borst connectors, barbed connectors, press-fit connectors, and/or Luer connectors.

As seen in FIG. 95, a filter 244B may also be located at, near, or within the lumen of the catheter inlet portion 244B. Further, as seen in FIG. 96, the distal end of the drainage catheter 116 may include a bypass lumen 232 to minimize the disruption of lymphatic fluid flow while the drainage catheter 116 is in place. This may be advantageous to allow native lymphatic flow if the filter were to become fouled or clogged and fluid could no longer pass through the catheter 116.

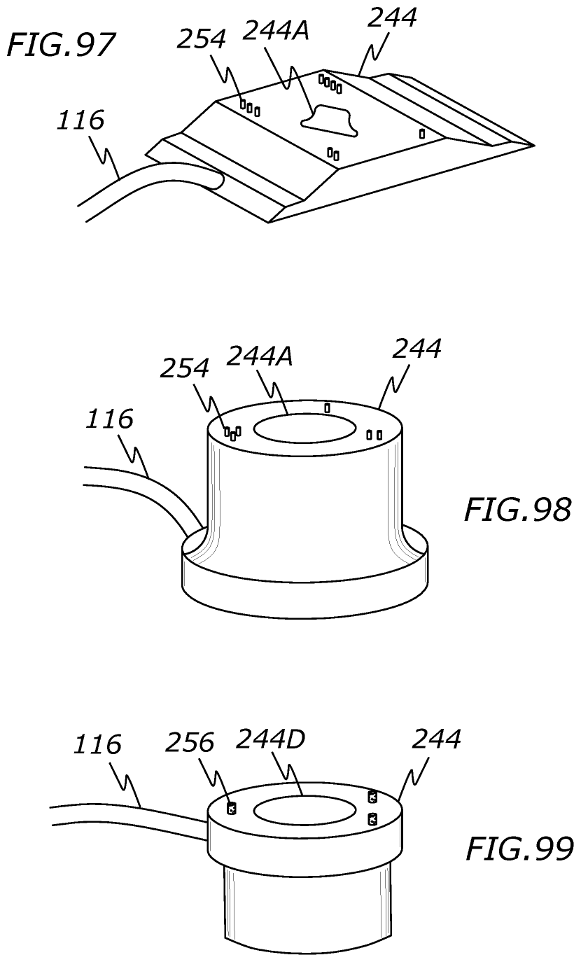

In some instances, it can be helpful to understand the exact location and orientation of the outlet portion 244A of the access port 244, especially when the access port 244 is located underneath the patient's skin. In this respect, the catheter access port 244 may contain one or more indicating tokens or indicia to aid in determining the location and/or orientation of the access port 244 and its outlet 244A. In one embodiment, an access port 244 may contain one or more features that may be detected by one or more of the following modalities including but not limited to tactile sensation, fluoroscopic imaging, X-ray, MRI, and ultrasound imaging. The features may include one or more of ribs, flats, holes, posts, extrusions, bosses, recesses, depressions, fillets, radii, tags, or markers. The ribs may have features to aid in determining the orientation of the port such as a different number of protrusions in different locations on the port or different concentrations of radiopaque materials in different locations on the port. For example, FIGS. 98 and 99 illustrate access ports 244 having several different groups of raised posts 254 positioned around the outlet portion 244A (e.g., a group of 1, 2, 3, and 4 posts 254). One of the groups of posts (e.g., the group of 3 posts 254 in FIG. 98) can be positioned closest to the catheter 116, thereby indicating where the catheter 116 connects to the access port 244. Alternately, two or more groups of posts 254 can be located at equal distances from the catheter 116 to indicate its position relative to the access port 244, as seen in FIG. 97.

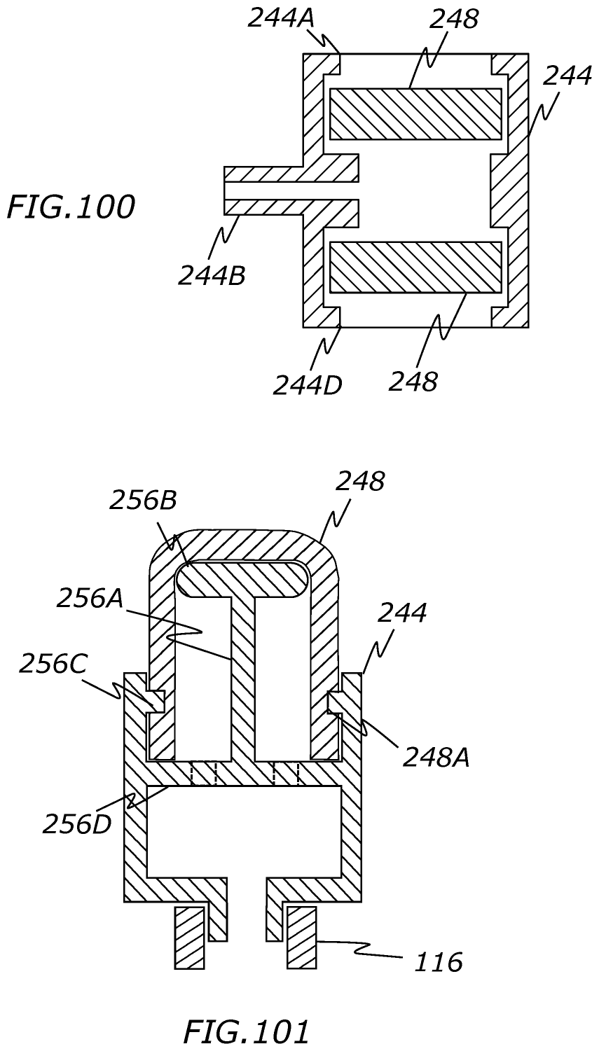

The access port 244 may also include one or more markers 256 on the bottom surface of the access port 244, as seen in FIG. 99, to detect whether the port has flipped over. These bottom markers 256 can be used alone or in addition to the previously described top markers 254. As also seen in FIGS. 99 and 100, the access port 244 may include another outlet 244D located on a bottom surface of the access port 244, along with its own sealing member 248, which allows drainage access even if the port 244 is flipped over relative to the orientation in which it was implanted (i.e., the bottom surface is facing towards the inside of the patient's skin).

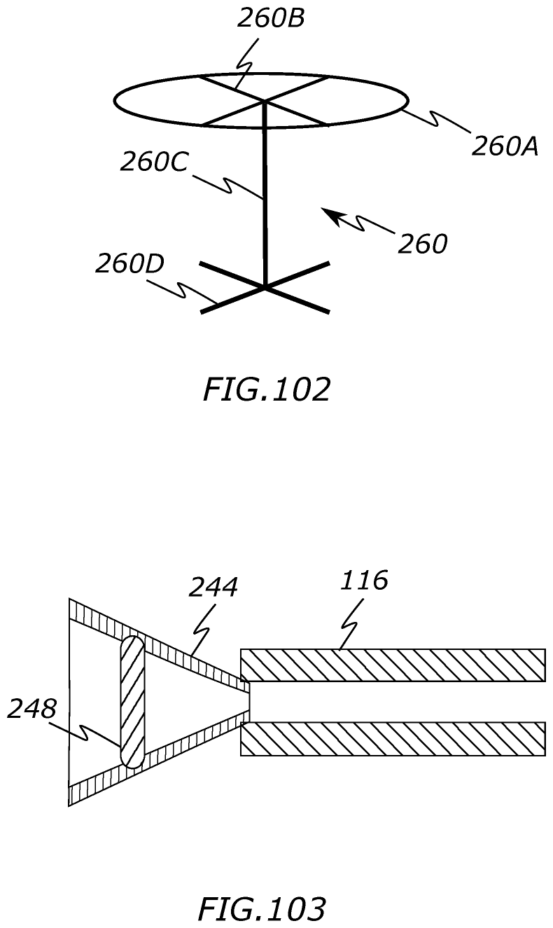

In another embodiment, an access port 244 may be comprised of a dome-shaped sealing member 248 to allow access to the interior of the port 244 at a plurality of angles and orientations. In one example seen in FIG. 101, the access port 244 includes a centrally-located, vertical post 256A that supports an enlarged shape 256B (e.g., a rounded disc shape) at its upper end. A lower end of the post 256A is connected to a ledge 256D that is connected to the body of port 244 and includes fluid passages therethrough. A ridge or lip 256C may also be included along the inner surface of the port 256C to engage a groove 248A along the outer surface of the sealing member 248, establishing fluid tight contact. Alternately, the sealing member 248 may be supported by a framework 260, shown in FIG. 102, having a wire ring 260A supported by a plurality of upper struts 260B to minimize the likelihood of an access device piercing the sealing member 248 and becoming stuck on the upper support structure. The upper struts 260B are fixed to an upper end of a vertical post 260C and a plurality of lower struts 260D. The lower struts 260D attached to the internal surface of the access port 244 by welding, adhesives, or may be integrally machined/molded to the port. The drainage catheter 116 may exit the port from a surface opposite the sealing member 248. Alternatively, the drainage catheter 116 may exit the port 244 from a surface that is perpendicular to the sealing member 248 (e.g. on a lateral surface of the port).

In another embodiment seen in FIG. 103, the access port 244 may be comprised of a cone or funnel shape that helps guide an access device toward a sealing member 248. The funnel shape may be additionally advantageous to allow the access device to be placed through the sealing member 248 and into the drainage catheter 116 without any intervening sharp turns. The port 244 may be radially symmetric (e.g., a circular cross section) or may be asymmetric along some cross sectional orientations (e.g., an oval cross section). The port 244 may also include attachment features on a flange (e.g., positioned on an outer surface of the conical shape) as described elsewhere in this application.

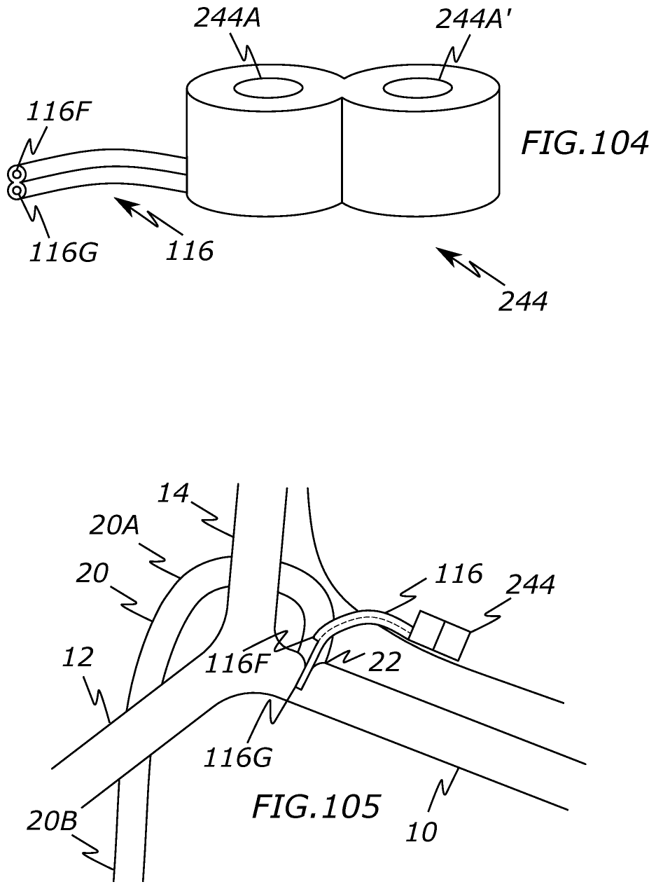

In one embodiment, a port 244 may be comprised of one or more separate access features connected to separate lumens in the drainage catheter 116 or separate drainage catheters 116. The distal end of the one or more lumens or one or more catheters may be in the same or different anatomical locations. For example, FIG. 104 illustrates an access port 244 having a first outlet 244A and a second outlet 244A′, each with their own sealing members. The first outlet 244A is in communication with a first catheter lumen 116F and the second outlet 244A′ is in communication with a second catheter lumen 116G. As seen in FIG. 105, the first catheter lumen 116F can open into a lymphatic structure (e.g., thoracic duct 20) and the second catheter lumen 116G may open into a venous vessel (e.g., left subclavian vein 10). This configuration may allow drainage from the lymphatic system while also allowing infusion of a fluid into the vein such as re-infusing filtered lymphatic fluid into the vein or infusing a fluid solution into the vein to maintain a desired electrolyte or protein balance in the body. Alternatively, the second catheter lumen 116G may provide a path for inserting a sensor for monitoring various physiological signals present in the vein without requiring a separate venous access site to be obtained. Alternatively, both lumens may be in a lymphatic vessel. This configuration may allow drainage from the lymphatic system while also allowing infusion of a fluid into the lymphatic vessel such as saline or heparinized saline to inhibit clot formation in the lymphatic fluid or re-infusing filtered lymphatic fluid or infusing a fluid solution to dilute the lymphatic fluid or a fluid solution to maintain a desired electrolyte or protein balance in the body. The infusion and drainage may occur simultaneously or at alternating times and frequencies.

In one embodiment, a valve 264 may be positioned between a catheter 116 and a connected access port 244 to selectively prevent fluid from flowing into the port 244 when draining is not desired and thereby preventing leakage from the port 244. The valve 264 may be maintained or biased to a closed position normally and opened by a user in the presence of an access member placed into the port 264. Alternately, the valve 264 may have an external control to adjust whether the valve 264 is open or closed. The valve 264 may be located in the distal, middle, or proximal portion of a catheter 116.

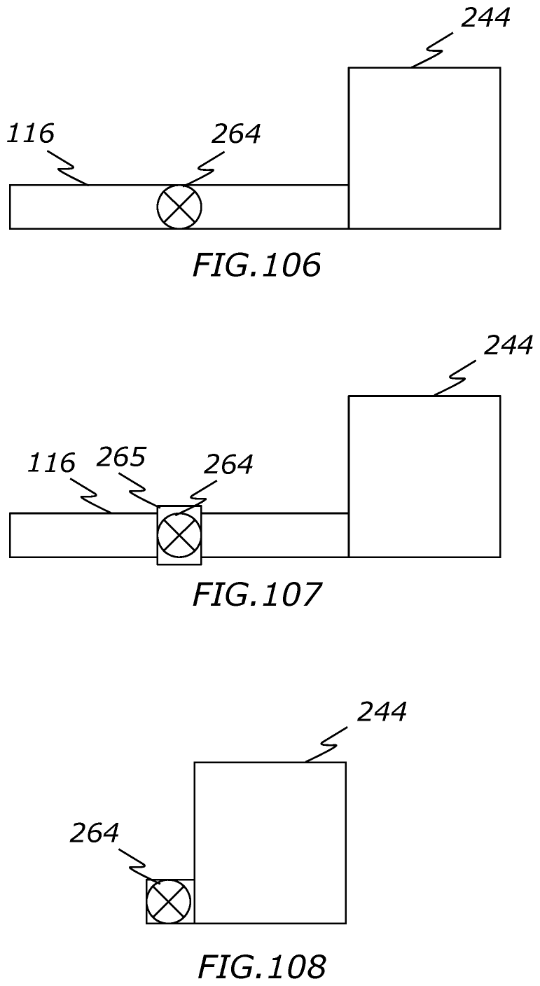

The valve 264 may be integrated into the catheter 116 (e.g., during manufacture) as seen in FIG. 106, included in a separate connector 265 that is placed in-line with the catheter 116 (e.g., prior to use) as seen in FIG. 107, can be integrated into the access port 244 (e.g., during manufacture) as seen in FIG. 108, or any combination of these locations. An in-line connector may be attached by any mechanism known to one skilled in the art, such as Luer-Lock, Tuohy-Borst, or a barbed connector and may be connected to the distal portion of the catheter 116.

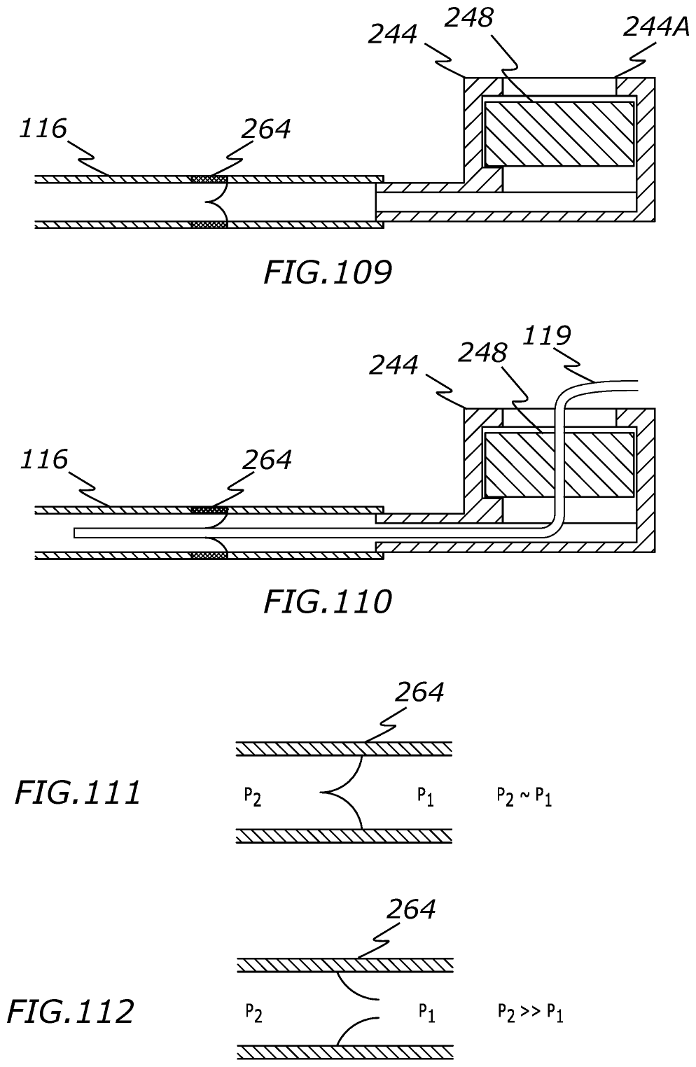

As seen in FIGS. 109 and 110, a second drainage catheter 119 can be advanced through the port 244, into the first catheter 116, and through the valve 264, causing the valve 264 to open.

The valve 264 may be one of any type of valve known to one skilled in the art, including but not limited to a duckbill valve, flapper valve, and check-valve. The valve 264 may be made from a flexible plastic material such as silicone, polyethylene, or urethane. Alternatively, instead of opening the valves with an access member (e.g., catheter 119), the one or more valves 264 may be forced open by a large pressure gradient in the direction of desired flow (from lymphatic vessel into the port 244). As seen in FIGS. 111 and 112, in the presence of a sufficient pressure gradient, the one or more valves 264 may invert and allow flow. Once the pressure gradient is reduced, the one or more valves 264 may revert and prevent flow. A sufficient pressure gradient may be created by accessing the port 244 and applying suction. A sufficient pressure gradient may be at least 500 mmHg, at least 250 mmHg, at least 100 mmHg or at least 50 mmHg.

Alternatively or additionally, the fluid path (e.g. drainage catheter or access member) may have a flow restrictor placed in-line rather than or in addition to a valve 264. The flow restrictor may introduce resistance to flow out of the lymphatic system (e.g., thoracic duct 20) and encourage at least some lymphatic fluid to remain in the thoracic duct 20 to help maintain a beneficial amount of electrolytes, white blood cells, or similar biological components. A flow restrictor may be particularly beneficial for patients with a relatively high pressure within a lymphatic structure and especially in the thoracic duct 20. For example, the flow restrictor may be an orifice plate or needle valve.

Alternatively or additionally, the flow restrictor may be comprised of a valve with a variable diameter orifice such as an iris valve such that the physician may change the size of the opening and, therefore, the amount of restriction introduced to obtain a desired flow rate through the drainage catheter. Alternatively or additionally, the flow restrictor may be comprised of a roller clamp attached to the exterior of the drainage catheter exterior to the patient's body. The physician may selectively engage the roller clamp to increase or decrease the resistance to flow to achieve a desired flow rate through the drainage catheter. Alternatively or additionally, a plurality of flow restrictors of varying resistances (e.g. orifice plates with different diameters) may be placed in parallel in the flow circuit (i.e. in an array in a manifold) and the physician may select a desired restrictor to achieve a desired flow rate through the drainage catheter. If a different flow rate is desired, the physician may switch to a different restrictor to increase or decrease the flow rate by, for example, opening the flow through one portion of the manifold and closing flow through another portion of the manifold.

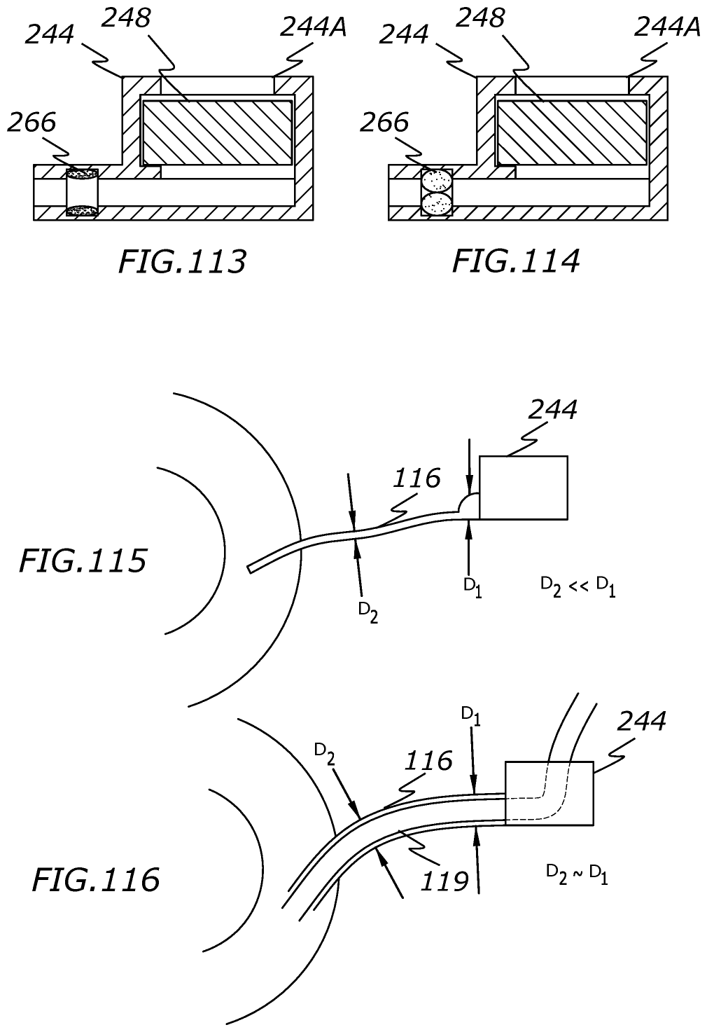

In one embodiment, an access port 244 may include an inflatable valve member to selectively prevent flow through the port 244. In the example shown in FIGS. 113 and 114, the inflatable valve member may be an inflatable balloon 266 located adjacent to or within the flow lumen of the port 244. Inflating the balloon 266 may partially or fully occlude the lumen and thereby prevent flow.

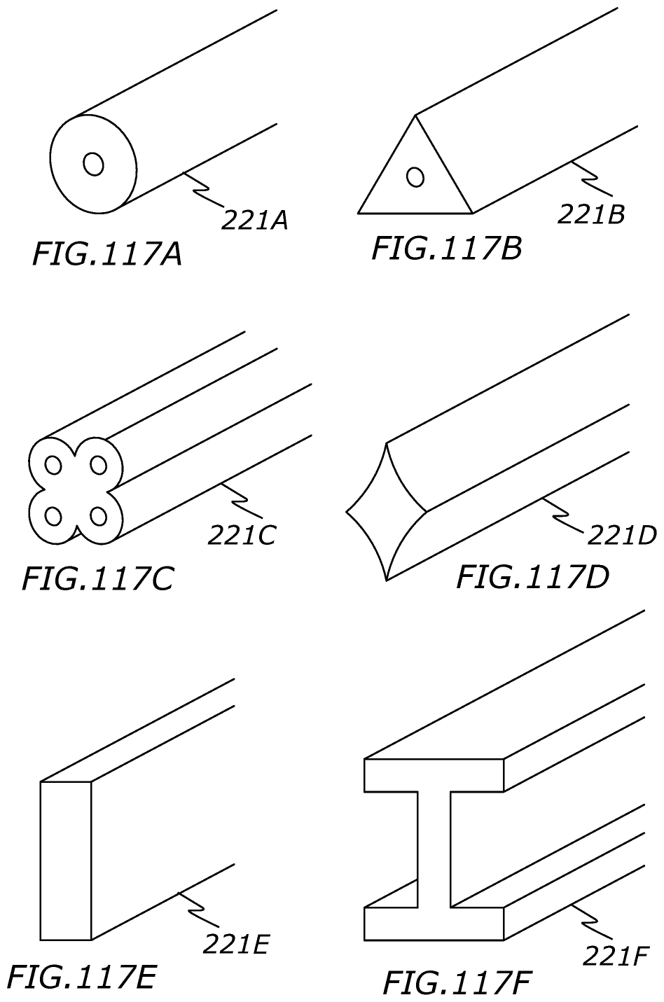

In one embodiment, an access catheter 116 connected to an access port 244 may be at least partially comprised of a flexible material, such as a silicone, that may at least partially radially collapse under typical conditions in a subcutaneous environment, as seen in FIG. 115. This may be advantageous to minimize the amount of fluid that could reside within the drainage catheter when drainage is not occurring. When drainage is desired, a second drainage catheter 119 with sufficient structural integrity (e.g., composed of a more rigid polymer) is advanced through the previously placed access catheter 116, causing the collapsed catheter 116 to radially expand. Alternately, a stylet may be placed through the collapsed access catheter 116 instead of or in conjunction with a drainage catheter 119 to radially prop open the access catheter 116. The stylet may be hollow, porous, or contain sufficient free space around its periphery to allow flow exterior to the stylet. In several specific examples, the stylet may have a cross sectional shape that is round (221A, FIG. 117A), triangular (221B, FIG. 117B), dual figure-8 shape (221C, FIG. 117C), diamond shape (221D, 117D), rectangular (221E, FIG. 117E), or I-beam shape (221F, FIG. 117F).

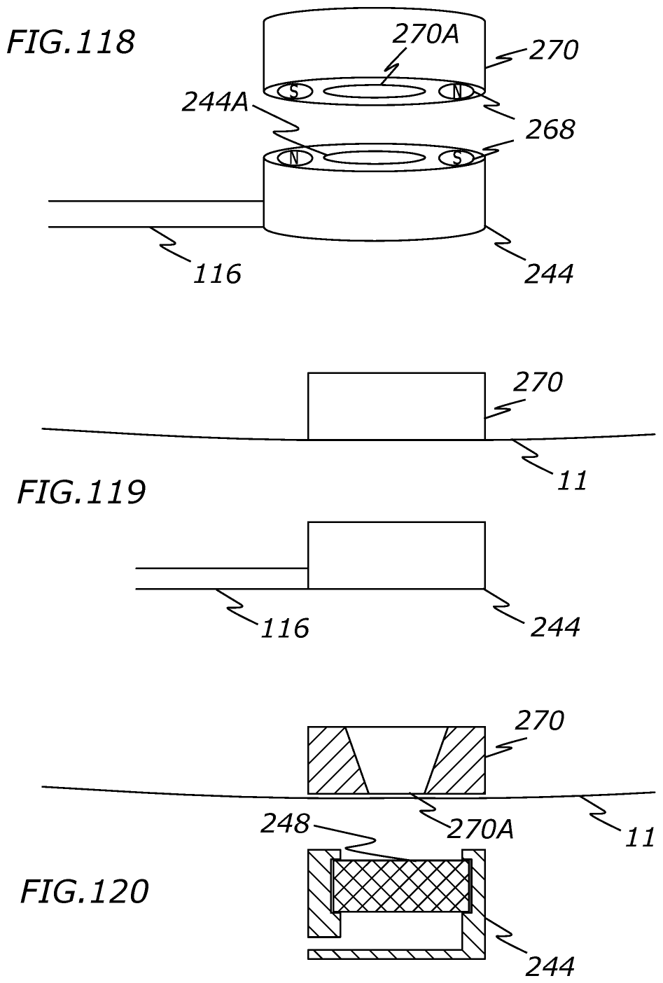

As previously discussed, it may be particularly difficult to locate an access port 244 and its outlet 244A when implanted underneath a patient's skin. In that regard, a coupling device 270 can be used to help locate and align with the access port 244 and outlet 244A. In one embodiment seen in FIGS. 118, 119, and 120, the access port 244 and the coupling device 270 may include a magnetic material, such as one or a plurality of magnets 268 located around the outlet 244A and lower opening 270A. The magnets 268 are configured to attract each other (e.g., north to south) and therefore align the two apertures 244A, 270A. Additionally, the plurality of magnets 268 can be arranged in a pattern to further help with alignment (e.g., alternating pole direction with adjacent magnets). The coupling device 270 generally includes a passage through it that aligns with outlet 244A of the port 244. The passage may have a generally uniform tubular shape or may be shaped to help direct an access device into the outlet 248A, such as the conical passage shape seen in FIG. 120.

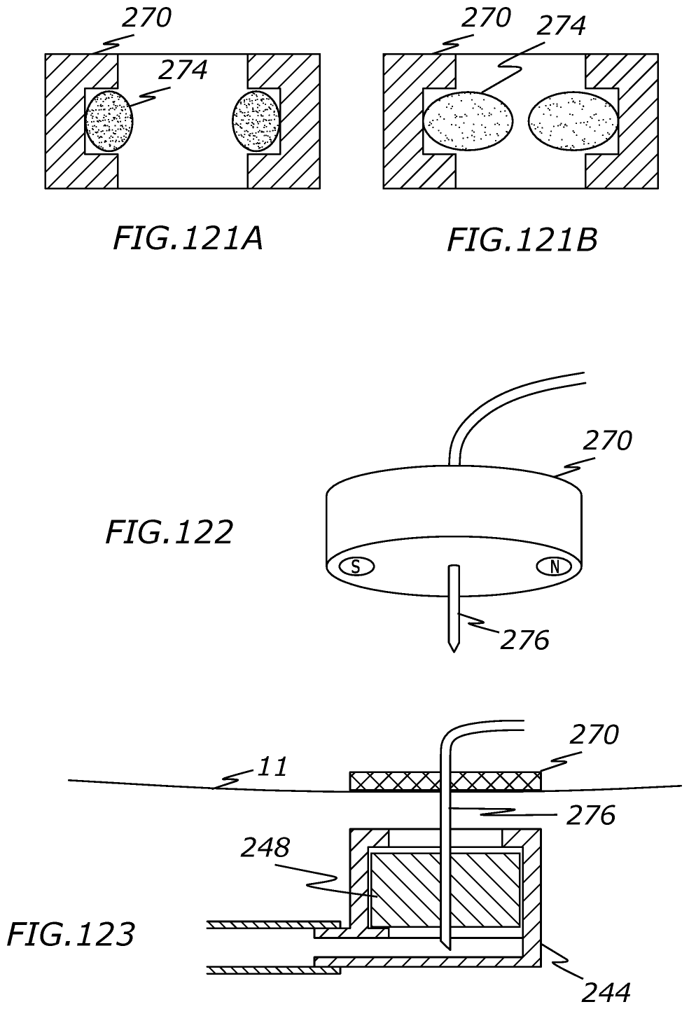

Additionally, a coupling device 270 may provide a mechanism of stabilizing an inserted access device (e.g., catheter 119). For example, FIGS. 121A and 121B illustrate a coupling device 270 having one or more selectively inflatable balloons 274 that are positioned at least partially around an inner surface of the coupling device 270. When deflated, the access device can freely move longitudinally through the coupling device 270 and when the balloon(s) 274 are inflated, they radially press against the access device, holding it in place. Alternately, the inner surface of the coupling device 270 may include a relatively large, resilient O-ring instead of the balloon 274 which instead can provide side support and a small amount of resistance to longitudinal movement.

In another embodiment shown in FIGS. 122 and 123, a needle 276 extends from downward from a lower surface of the coupling device 270. The needle 276 may be either permanently or removably fixed to extend to a predetermined depth suitable for passing into the access port 244 and through the sealing member 248 to thereby access the catheter 116. In one embodiment, the coupling device 270 is aligned with the access port 244 and, once alignment is achieved, the needle 276 can be advanced into the coupling device 270 (if it is not already partially positioned within the coupling device 270), through the skin 11, and into the access port 244. In one embodiment, the coupling device 270 includes a passage having a diameter large enough to allow the needle 276 to longitudinally slide through. This may be advantageous to allow the needle 276 to be retracted or completely removed while the coupling device 270 is aligned with the access port 244 and, once alignment is achieved, the needle 276 can be advanced through the skin 11 and into the access port 244. Optionally, a clamping mechanism may also be included on the coupling device 270 to selectively fix the needle 276 in place. The coupling device may also contain a filter, similar to those previously discussed, to remove only the desired constituents of the fluid.

While the access port 244 may be accessed by any type of needle, a non-coring needle, such as a blunt tip or Huber tip needle, may be preferred in any of the embodiments of this specification where the access port 244 is intended to be accessed in multiple instances. Since coring needles may remove small amounts of the sealing member 248 during each insertion, they may create a gap or openings around the needle after multiple insertions, thereby causing leakage. The non-coring needle may have a bend to prevent the back edge of the lumen of the needle from contacting the sealing member 248 in the access port 244 as it is being inserted (e.g., a Huber point needle).

One method of removing lymphatic fluid may utilize a drainage device system including a needle 276, an access port 244, and a catheter 116 attached to the access port 244 with a distal end located in a lymphatic structure. In this method, the needle 276 is first placed into the access port 244. The needle 276 may pass through a sealing member/assembly to establish a fluid path from the lumen of the needle 276 into the port 244 and which establishes communication with the lumen of the attached catheter 116 and lymphatic structure. Lymphatic fluid can then be drawn out of the needle 276 and drainage is achieved.

Another method of removing lymphatic fluid may comprise using a drainage device system including a needle 276, guidewire, a drainage catheter 119, and port 244, and a catheter 116 attached to the access port 244 with a distal end located in a lymphatic structure. In this method, the needle 276 is placed into the port 244 and the guidewire is placed through the lumen of the needle 276. The needle 276 is removed with the guidewire remaining in place. The drainage catheter 119 is advanced over the guidewire and into the port 244 and coupled with the catheter 116. The drainage catheter 119 is advanced as needed until access to the lymphatic fluid is established. Lymphatic fluid can then be drawn out of the drainage catheter 119 and drainage is achieved.

With any of the aforementioned methods, if desired, suction may be applied to the needle 276 or drainage catheter 119 to increase the pressure gradient to enhance the flow of lymphatic fluid.

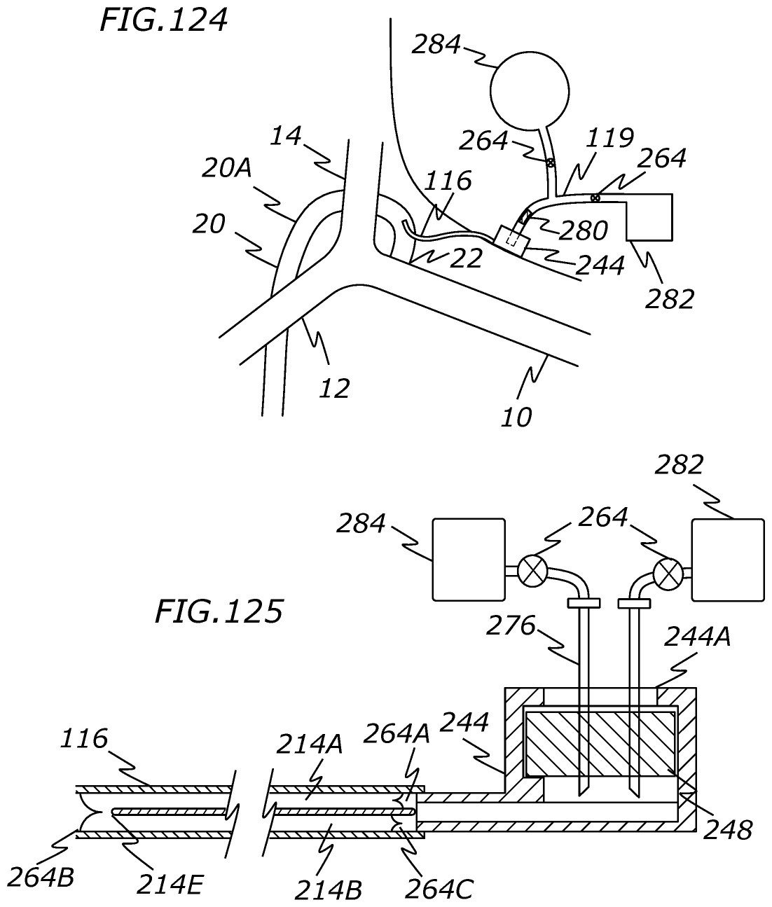

If fluid removal slows or stops during drainage, it is possible that a clog is present in the system. In order to dislodge a clog, one method may include reversing the direction of the flow in the drainage system. Fluid (e.g. saline, isotonic fluid) may be pushed into the drainage system (e.g., needle 276, catheters 116, 119, and/or port 244) for a short period of time and then stopped to allow drainage to resume and determine if the drainage flow rate increased. One example of such a system can be seen in FIG. 124, in which a catheter 119 is coupled to a port 244 and implanted catheter 116. The catheter 119 is connected to both a reservoir 282 and to a fluid source 284 capable of supplying fluid at a relatively high pressure back into the catheter 119. Both the reservoir 282 and the fluid source 284 may each have valves 264 that are configured to allow drainage into the reservoir 282 and close off access to the fluid source 284 in one operational state and close off the reservoir 282 and open up the fluid source 284 in another operational state. Hence, fluid from the fluid source 284 can be pushed into the catheter 119 towards catheter 116 without moving into the reservoir 282. The valves 264 may be manually opened/closed by hand, electronically actuated, or may be configured to automatically open/close at the appropriate direction of pressure (e.g., a one-way valve).

Additionally, a flow sensor 280 may be included in the drainage catheter 119 to detect a decrease in the drainage flow rate and automatically open a valve and employ the above method to remove a clog. For example, the flow sensor 280 in FIG. 124 can be located in catheter 119, however, sensors in catheter 116 or access port 244 are also possible. The system may start with the valve 264 to the fluid source 284 closed and the valve 264 to the drainage reservoir 282 open to allow drainage flow into the reservoir. If the flow sensor 280 senses a reduction or cessation of fluid flow, the valve 264 to the drainage reservoir 282 may close (manually or via electronically actuated control) and the valve 264 to the fluid source 284 may open. If the fluid source 284 is not already under relatively high pressure, such pressure can then be generated to cause fluid from the fluid source 284 to flow into the port 244 and catheter 116, dislodging a potential clog. After a short period of time, the valves 264 may reset to their prior open/closed states to allow normal drainage flow. The flow sensor 280 may continue to measure the fluid flow to determine if the clog has been resolved. If not, another flush cycle may be performed, or the sensor 280 and its accompanying control system may notify the user of a potential issue.

In one embodiment, the drainage system can be configured to allow the fluid source 284 to inject fluid and cause circulation within the lumens of the system. One example of this can be seen in FIG. 125 in which the fluid source 284 and drainage reservoir 282 are connected to the access port 244 via two different needles 276 and/or catheters 119. Fluid such as saline or heparinized saline injected into the system by the fluid source 284 is configured to enter a first catheter lumen 214A at its proximal end, travel to a distal end of the catheter 116 to a connection area 214E, enter the second catheter lumen 214B, and then travel back toward a proximal end of the second catheter lumen 214B. The fluid injection and removal may be alternated one or more times to allow the infused fluid to fill the lumens prior to removing the fluid.

Additionally or alternatively, the port 248 may have two separate sealing members, outlets, and independent chambers and fluid paths for the first catheter lumen 214A and the second catheter lumen 214B as shown in FIG. 104. This may be advantageous to allow simultaneous infusion and removal of fluid and prevent short-circuiting of fluid directly from the infusion needle into the drainage. Additionally, with any embodiment, it may be beneficial to flush and fill the lumens with a fluid such as saline or heparinized saline at the end of a therapy session to fill the lumens and displace any lymphatic fluid to prevent it from clotting and clogging in between sessions.

This fluid flushing path can be directed by a plurality of one-way valves 264. For example, the first catheter lumen 214A may include a one-way valve 264 in the first catheter lumen 214A to allow fluid flow in a distal direction and the second catheter lumen 214B includes a second one-way valve 264C in the second catheter lumen 214C to allow fluid flow in a proximal direction. A single one-way valve 264B can also be included at the distal end or opening of the catheter 116 to prevent fluid flushed in by the fluid source 284 from exiting the distal end of the catheter 116 but allowing drainage from the lymph structure into the catheter 116. Additionally, the fluid source 284 may include a one-way valve 264 configured to allow only the release of fluid into the access port 248 while the reservoir 282 may include a one-way valve 264 configured to allow fluid from the access port 248 to only pass into the reservoir 282.

In this respect, during drainage, fluid may enter the drainage catheter 116 and pass through the second lumen 214B to the access port 248 and be removed by a first access member connected to a reservoir. If the lumen requires flushing, a second access needle/catheter may be inserted into the access port 248. The second needle/catheter may provide fluid flow (e.g. water, saline, lactated ringer's solution, etc.) from the pressurized fluid source 284 (e.g., a syringe, a fluid bag placed above the port, or a fluid pump). The fluid may pass through the first lumen 214A down to the distal end where the pressure may force the distal valve 264B to close and prevent the fluid from exiting the drainage catheter 116. The fluid may then follow the fluid circuit through the first lumen 214B and into the port 248. The fluid may then exit through the first access member.

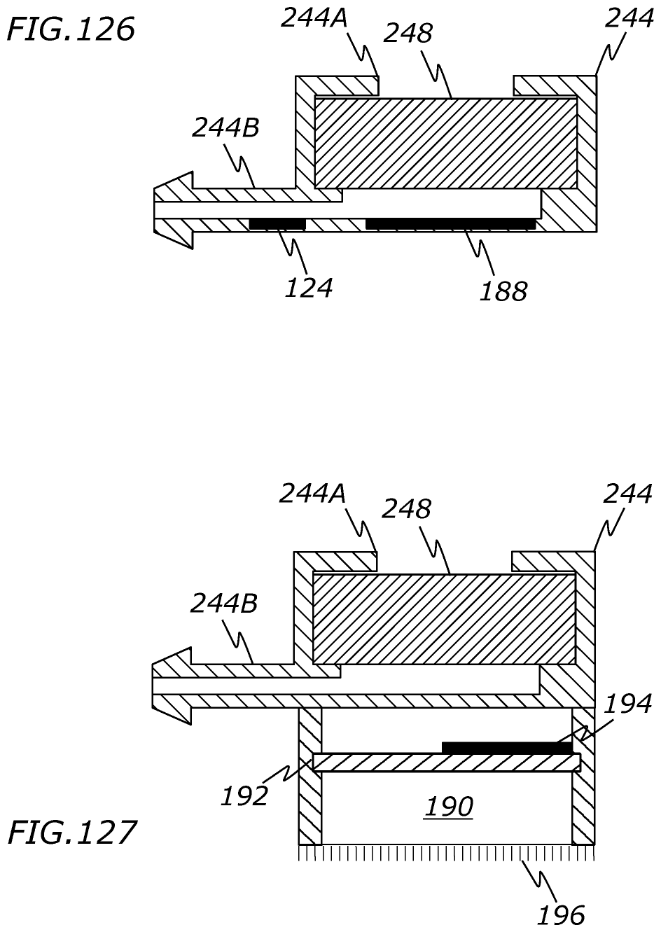

In one embodiment seen in FIG. 126, an access port 244 may contain one or more sensors 124 and a wireless transceiver assembly 188 configured for connecting to and communicating sensor data to external devices. The one or more sensors 124 may sense one or more of the following signals such as pressure, flow rate, pH, salinity, and/or temperature. The wireless transceiver assembly 188 may include a microprocessor processor to receive, process, and otherwise format sensor data into a form suitable for wireless transmission, a wireless transceiver (e.g., Wi-Fi, Bluetooth, cellular, etc.), and an antenna. During use, a patient may place an external device near the port 244 to receive and monitor the sensor data during drainage, such as the pressure in the port 244 and/or the flow rate through the port 244.

In one embodiment, an access port 244 may contain an integrated pressure sensor for measuring pressure outside of the port 244, such as in the interstitial space of a patient if the port 244 is implanted. One example can be seen in FIG. 127 in which the access port 244 includes a lower chamber 190 that is in communication with a microneedle array 196 on the bottom surface of the port 244, which thereby provide a plurality of openings in which pressure can be communicated into the lower chamber 190. The lower chamber 190 further includes a flexible diaphragm connected to each wall of the lower chamber 190, effectively sealing this chamber 190 into two portions. A strain sensor 194 is positioned on the upper surface of the diaphragm 192, allowing it to measure the amount the diaphragm 192 deflects as pressure within the chamber 190 (from outside the access port 244) changes. Additionally, the microneedle array 196 may help maintain the relative location of the port 244 within the body after implantation. The port 244 can include a wireless transceiver assembly 188 as previously described to communicate this pressure data to a nearby device for monitoring and/or storage.

Any of the aforementioned inventions may be employed to drain lymphatic fluid from a patient in a single session (i.e. one drainage therapy) or in more than one separate sessions (i.e. multiple drainage therapies). The separate sessions may be separated in time by hours, days, weeks, months, or years.

As discussed in the methods and embodiments below, the lymphatic fluid can be drained from a lymphatic structure into a reservoir located within the body, where it is stored for later external removal. For simplicity, the thoracic duct 20 will be used as an example of a lymphatic structure; however, any lymphatic structure can be used without deviating from the present invention. It should be appreciated that any of the embodiments related to draining lymphatic fluid may be combined with any of the present embodiments by including a reservoir at any point in the fluid path of the drainage system.

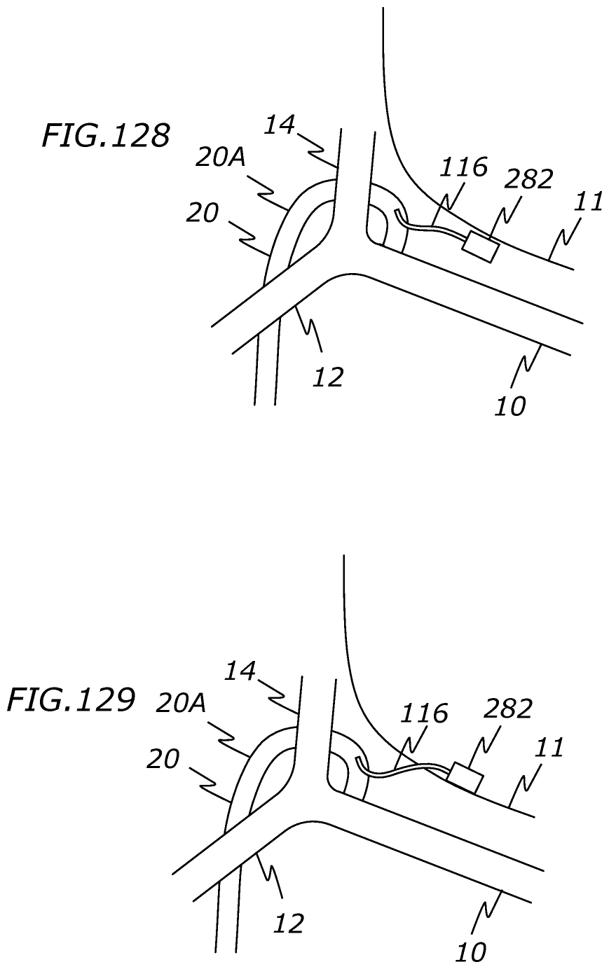

In one embodiment, a reservoir 282 may be connected to a proximal end of a catheter 116 whose distal end is positioned a lymphatic vessel to allow the accumulation of lymphatic fluid within the reservoir 282. The reservoir 282 may be implanted underneath the skin 11, as seen in FIG. 128, in any suitable anatomical location including but not limited to the chest, abdomen, thigh, bladder, colon, back, shoulder. Alternatively, the reservoir 282 may be placed external to the skin 11, as seen in FIG. 129, with the catheter 116 traversing the skin 11. The subcutaneous reservoir 282 may provide a fluid storage area that is more discreet and aesthetically pleasing to the patient, as well as more difficult to accidentally pull out or dislodge. Conversely, the external reservoir 282 may be easier to access to remove accumulated fluid since it is easier to view and manipulate.

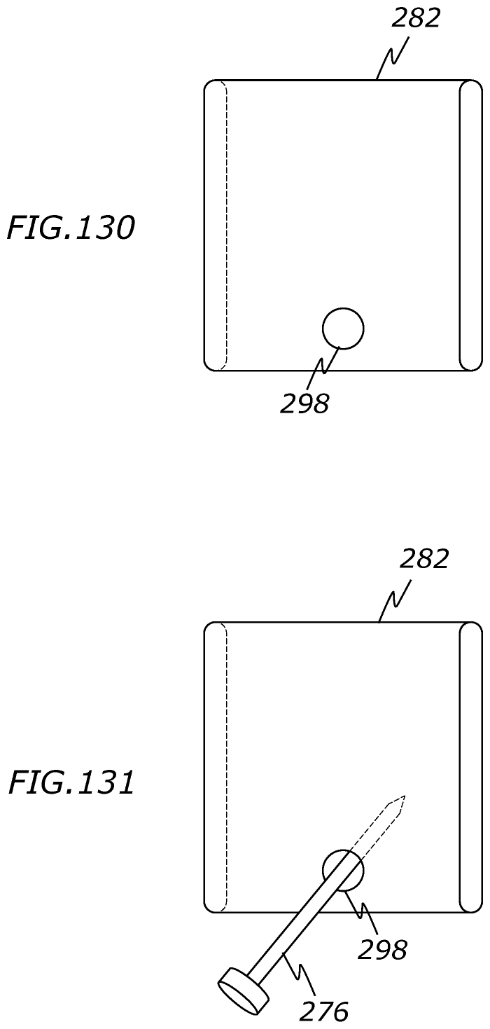

The reservoir 282 may contain a septum to allow direct piercing with a needle 276 to drain, as seen in FIGS. 130 and 131. The septum may be made from a self-healing material such as silicone or rubber to allow repeated piercings without leaking. With regard to a subcutaneous reservoir 282, it should be noted that the septum 298 can be further configured in any manner previously discussed with regard to a subcutaneous access port 244. For example, the septum 298 may include raised features, magnets, coupling devices, or similar mechanisms to help locate and access the septum 298.

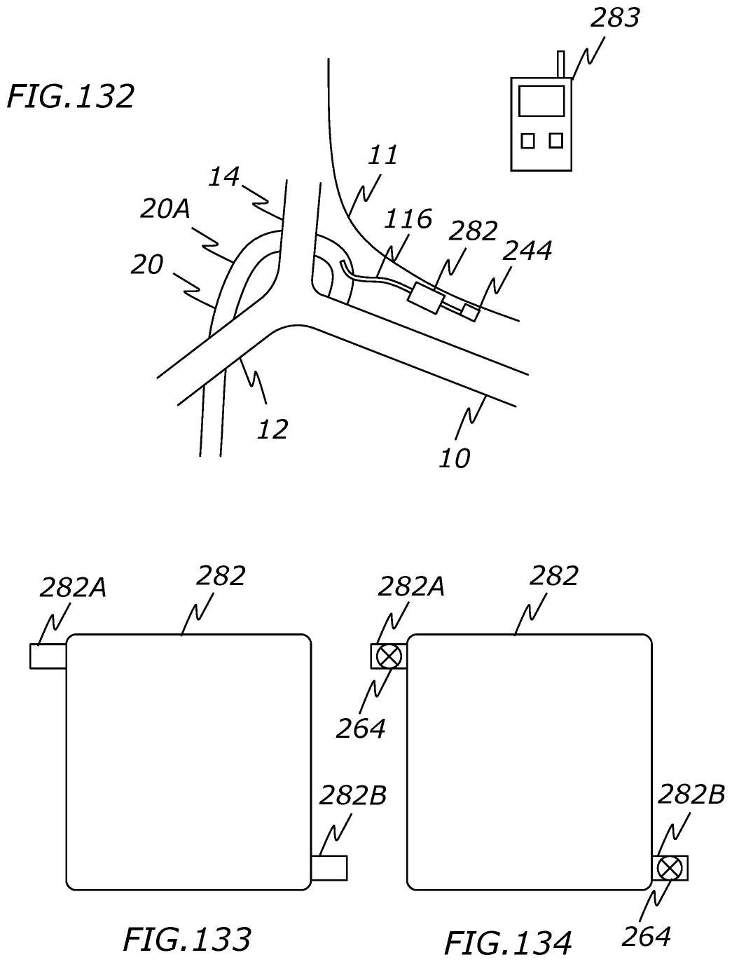

In another embodiment seen in FIG. 132, a reservoir 282 may be connected between a catheter 116 and an access port 244 to allow the accumulation of lymphatic fluid in the reservoir 282. The contents of the reservoir 282 may then be removed by accessing the port 244 and draining the fluid. The reservoir 282 and port 244 may be placed under the skin 11 or above the skin 11.

As seen in FIG. 133, the reservoir 282 may further be comprised of one or more inlet conduits 282A and outlet conduits 282B that can be connected to a catheter 116 and access port 244, respectively. The inlet conduit 282A and outlet conduit 282B may also include of one or more valves 282 as seen in FIG. 134 to control the flow of fluid into and out of the reservoir 282 (e.g., prevent backflow into the catheter 116). The valves 264 may be one-way valves such as duckbill valves or bi-directional such as butterfly valves or a gate valves. The valves may also be biased to a closed position to prevent flow without actuating the valve 264. The valves 264 may also be momentary valves that are only open in the presence of a stimulus such as pressure, torque, or magnetic force.

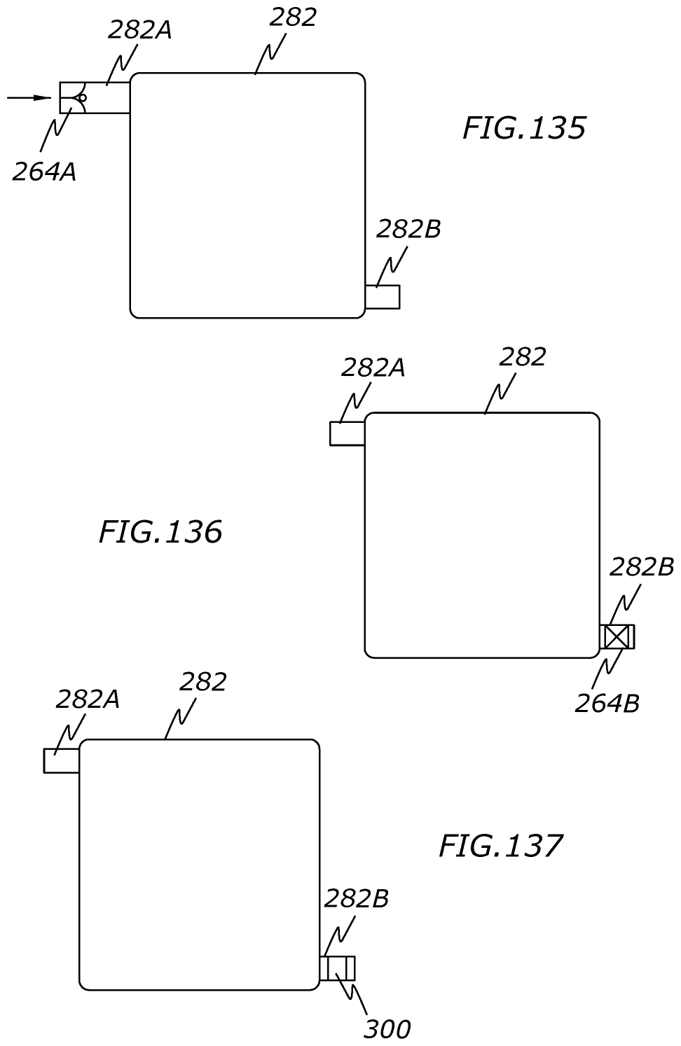

As seen in the example of FIG. 135, the, the inlet conduit 282A may contain a one-way valve 264A to allow fluid to enter but not leave through the inlet conduit 282A to prevent retrograde flow. In another embodiment seen in FIG. 136, the outlet conduit 282B may contain a gate valve 264B that prevents fluid from passing in either direction without the valve 264B being actuated.

The valves 264 may be actuated by external means provided by the patient, such as magnetic input (e.g., placing a magnet close to the valves 264) or tactile input (e.g. pushing a button). For example, when the patient is ready to empty the reservoir 282, the patient may place a magnet over the valve 264 in the outlet conduit 282B to allow flow out of the reservoir 282. Alternatively, the one or more valves 264 may include solenoids that are powered by electrical or electromagnetic means. The patient may press a button on a controller 283 (e.g., as seen in FIG. 132) to open or close the one or more valves 264 at a desired time. In one embodiment, the controller 283 may include a processor, memory accessible by the processor, software stored in the memory, a communication component (e.g., a wireless transceiver and antenna), and a user input. In one example, the controller 283 may be a smart phone or a unit dedicated for use only with the reservoir 283.

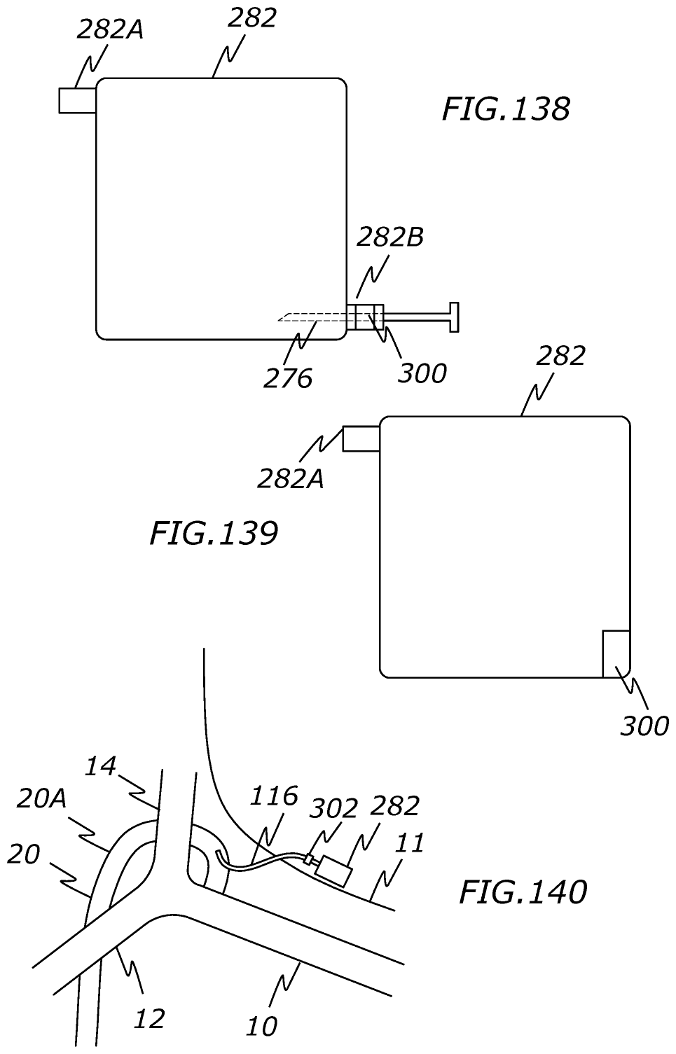

Alternatively, the outlet conduit 282B may include a septum 300 to allow access without actuating a valve 264, as seen in FIG. 137. In order to empty the reservoir 282, the patient may place a needle 276 across the septum 300 to access the fluid and allow drainage through the needle 276, as seen in FIG. 138. Additionally, the septum 300 and outlet conduit 282B may be formed in an integral manner with the body of the reservoir 282 so as to form a generally uniform surface, as seen in FIG. 139.

Additionally, the reservoir 282 may contain one or more sensors including but not limited to pressure sensors, pH sensors, strain gauges, temperature sensors, weight sensors, tension sensor, magnetic sensor, distance sensors, flow sensors, and optical sensors. These sensors can communicate with other devices via a wired electrical connection or via a wireless transceiver assembly similar to those previously described.

In one embodiment seen in FIG. 140, the reservoir 282 may contain only an inlet conduit with a connector 302 to the catheter 116. The connector 302 may allow for fluid flow in both directions, such as a Luer-Lock or barbed connector, or may only allow flow into the reservoir 282, such as a Luer-Lock with an integrated one-way valve. Alternatively or additionally, the reservoir 282 may contain an integral one-way valve to prevent fluid from flowing back into catheter 116. When the reservoir 282 is full, the patient may disconnect and discard the reservoir 282 and attach a new reservoir 282.

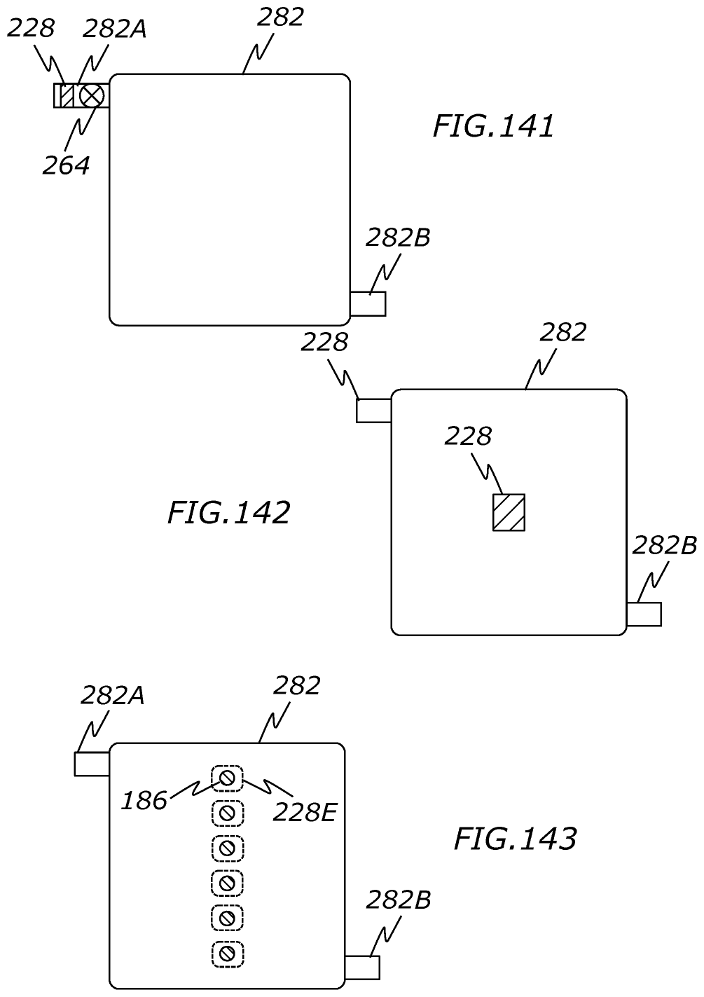

In one embodiment seen in FIG. 141, the reservoir 282 may include a sensor 228 (e.g., a pressure sensor or flow sensor) and valve 264 in the inlet conduit 282A. If the sensor 228 is a pressure sensor, it may provide pressure data to a control mechanism (e.g., microprocessor) to cause the valve 264 to open when the sensed pressure exceeds a specified threshold. The pressure sensor may then close the valve 264 when the pressure falls to below a specified threshold. The opening and closing thresholds may be the same or the thresholds may be different (e.g. one threshold may be higher than the other). In another embodiment, the sensor 228 provides pressure data to a control mechanism (e.g., microprocessor) which notifies the patient if the sensed pressure value exceeds a predetermined threshold, allowing the patient to open the inlet valve 264 to allow fluid flow.

In one embodiment, the reservoir 282 may be comprised of an inlet pressure sensor 228 and valves 264 on the inlet 282A and outlet conduits 282B. The pressure sensor 228 (or rather a control system with a microprocessor monitoring the pressure data) may notify the patient when a pressure threshold is exceeded, and the patient may open the valve 264 on the inlet conduit 282A to start filling of the reservoir 282. The patient may then open the valve 264 on the outlet conduit 282B at a convenient time and in a discrete location. After emptying the reservoir 282, the patient may close both valves 264.

In one embodiment seen in FIG. 142, the reservoir 282 may include a sensor 228 located within the reservoir's interior fluid holding lumen. In one embodiment, the sensor 228 may sense a fill level of the reservoir 282 and be comprised of a strain gauge sensor that monitors stretching the reservoir's fluid holding lumen material to determine how full the reservoir 282 has become. When the reservoir's fluid level exceeds a threshold, such as 50%, 75%, or 100% of full capacity, the sensor 228 (or rather a control system monitoring the sensor data) may notify the patient so the patient may drain the reservoir 282.

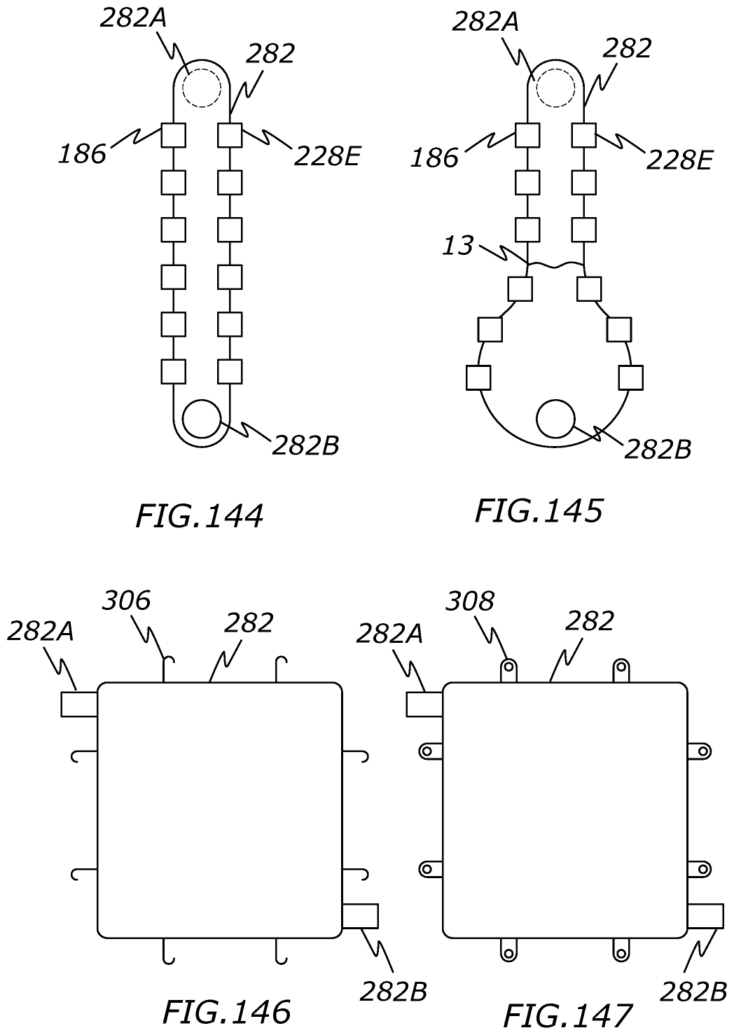

FIGS. 143-145 illustrate another example of sensor system configured to detect a fill level of the reservoir 282. Specifically, the reservoir 282 may contain a plurality of magnets 186 arranged in a longitudinal array along a first side of the reservoir 282. A second, opposite side of the reservoir 282 may include a longitudinal array of a plurality of magnetic field sensors 228E that are configured to measure the magnitude of a nearby magnetic field. As seen best in the end views of FIGS. 144 and 145, as fluid 13 is introduced in the reservoir 282, it moves the magnets 186 away from the magnetic field sensors 228E. A microprocessor (either integrated in the reservoir 282 or in a separate control device) monitors which sensors 228E have reduced magnetic field values and thereby determines that those areas of the reservoir 282 contain fluid 13. Hence, the microprocessor and its executed software algorithm can determine how full (e.g., a percentage, such as 25%, 50%, or 100%) the reservoir 282 has become. The microprocessor can cause this information to be displayed or otherwise communicated to a patient or medical professional (e.g., a full reservoir alert).

To ensure that any spacing between the magnets 186 and sensors 228E are due to the presence of fluid 13 and not other external forces deforming the shape of the reservoir 282, it may be helpful that the reservoir 282 is biased to a shape where its walls press against each other or are otherwise close to each other. This can be accomplished, for example, by molding a bias shape into the polymer materials of the reservoir 282, including curved structural members in the walls of the reservoir 282, or including ferrous metal in the reservoir wall opposite that magnets 186 so that the magnets 186 are attracted to ferrous metal, or including a second set of magnets 186 in the reservoir wall opposite the first array of magnets 186.

While the magnets 186 and sensors 228E are shown as arrays along similar/parallel vectors, additional arrays of magnets 186 and sensors 228E along other vectors (e.g., perpendicular vectors) may also be possible. Such additional arrays may be helpful in measuring a fill level when the reservoir 282 is positioned on its side or at an angle.

In another embodiment, the reservoir 282 may be comprised of an inlet pressure sensor and fill sensor as described elsewhere in the application and valves 264 on the inlet 282A and outlet conduits 282B. The control system (e.g., microprocessor) monitoring the pressure data may notify the patient when a pressure threshold is exceeded, and the patient may open the valve 262 on the inlet conduit 282A to start filling of the reservoir. The control system monitoring data from the fill sensor 228 may the notify the patient when a desired amount of volume has been filled in the reservoir 282, and the patient may then open the valve 264 on the outlet conduit 282B to empty the reservoir 282. The reservoir 282 may contain a buffer volume beyond the desired amount of volume to be removed to allow the patient to empty the reservoir 282 at a convenient time and in a discrete location. After emptying the reservoir 282, the patient may close both valves 264.

In another embodiment, the reservoir 282 may be comprised of a fill sensor as described elsewhere in this application and a valve 264 at the outlet conduit 282B. The reservoir 282 may be continuously filling and when the fill sensor detects that the volume in the reservoir 282 exceeds a threshold, a control system monitoring the sensor data may notify the patient to empty the reservoir 282. Additionally, the reservoir 282 may be comprised of a check valve on the inlet conduit 282A to only allow filling when the inlet pressure exceeds the cracking pressure of the check valve such as 10 mmHg, 15 mmHg, or 20 mmHg.

In any of the embodiments, the reservoir 282, conduits 282A, 282B, and septa 300 may be composed of a soft plastic material such as polyethylene, polyurethane, silicone, rubber, or polyvinyl chloride (PVC) in a thickness of 0.02″, 0.01″, 0.005″, 0.003″, or 0.001″. The soft plastic material may allow the reservoir to expand as it fills without a significant increase in pressure until the reservoir is full. The plastic material may contain one or more of the following constituents including but not limited to an antimicrobial additive, a radiopaque additive, a hydrophilic additive, a hydrophobic additive, or a plasticizer.

The reservoir 282 may also include attachment mechanisms for securing to the patient. For example, the attachment mechanism may be comprised of hooks 306 to attach to the patient directly (FIG. 146) or loops or outwardly extending structures with apertures 308 to use in conjunction with securement means such as suture, staples, or clips (FIG. 147). The apertures 308 may be reinforced with thicker plastic or metal grommets. Alternatively, the reservoir 282 may have an adhesive backing to attach to the patient. The reservoir 282 may be manufactured using an RF-welding process, ultrasonic welding process, solvent bonding process, heat bonding, or adhesive bonding process.

In one embodiment, the reservoir 282 may be comprised of one or more inlet valves that are controlled by a controller and communication device. The controller may open and close the inlet valves based on a timing sequence such as open for 4 hours then closed for 20 hours or open for 12 hours and then closed for 12 hours. The control algorithm may be based on a 24-hour cycle, a shorter cycle, or a longer cycle. The control algorithm may be prescribed and programmed by a physician. The controller may access a wireless communication device to allow remote programming.

In one embodiment, the reservoir 282 may be comprised of one or more inlet valves 264 that are controlled by a controller and communication device in communication with one or more sensors. The controller may open and close the inlet valves 264 based on a reading from a flow sensor and pressure sensor at the inlet of the reservoir 282. The controller may open the inlet valve 264 when the pressure exceeds a given threshold. The inlet valve 264 may then be closed after a given volume of fluid that passed into the reservoir 282 (e.g. based on integrating the flow sensor flow rate signal or using a fill sensor) or when the desired flow rate of fluid into the reservoir 282 has been reached or when the pressure reaches a threshold. The control algorithm may be determined and programmed by a physician. The controller may access a wireless communication device to allow remote programming.

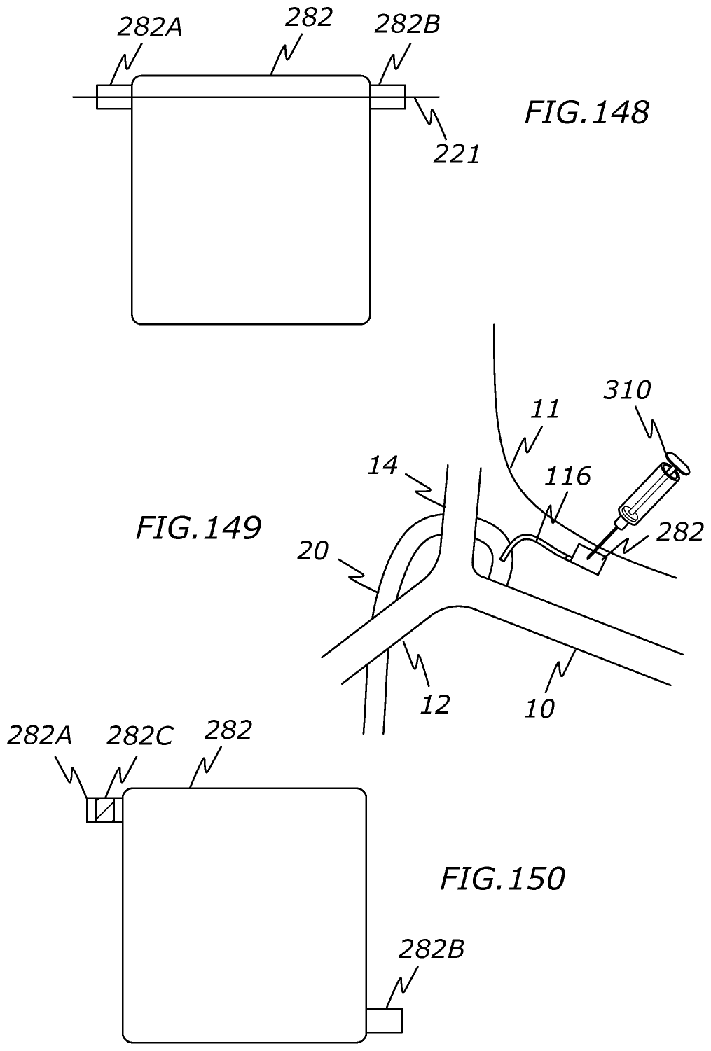

In one embodiment, the inlet conduit 282A and outlet conduit 282B are attached to the reservoir 282 with connection mechanisms that allow them to be disconnected from the reservoir 282, such as Luer Lock connectors, Tuohy Borst connectors, or barbed connectors. If either of the inlet conduit 282A or outlet conduit 282B become clogged, the conduit may be disconnected from the reservoir 282 so that it can be cleaned or replaced. As seen in FIG. 148, the inlet conduit 282A and outlet conduit 282B may alternately be positioned so that their passages align with each other to allow a cleaning device such as a stylet 221 to be introduced into the outlet conduit 282B and pass through the reservoir 282 to the inlet conduit 282A to free any blockages. The stylet 221 may have bristles, protrusions, or any additional features to aid in clearing blockages.

In one embodiment, fluid in the reservoir 282 may be removed with a needle and syringe 310, as seen in FIG. 149. When connected to a drainage device (e.g., catheter 116 and reservoir 282), the syringe plunger may be pulled back to remove lymphatic fluid or generate a vacuum to initiate or enhance the removal of lymphatic fluid. Additionally, the syringe 310 may be comprised of a position lock (e.g., a VacLok from Merit Medical) to allow the syringe plunger to be pulled back a desired amount and maintain that position to generate a desired suction pressure if the resistance to flow is great enough or to remove a desired amount of fluid. The position lock may also limit the amount of fluid removed by creating a closed system with a fixed volume since the fluid is substantially incompressible. After the syringe 310 is filled with the desired volume of fluid, the needle may be withdrawn and the syringe 310 may be discarded. An additional syringe can then be attached to remove additional fluid.

In one embodiment seen in FIG. 150, the reservoir 282 may include a filter 282C in an inlet conduit 282A so that only the desired constituents of the lymphatic fluid are drained into the reservoir 282.

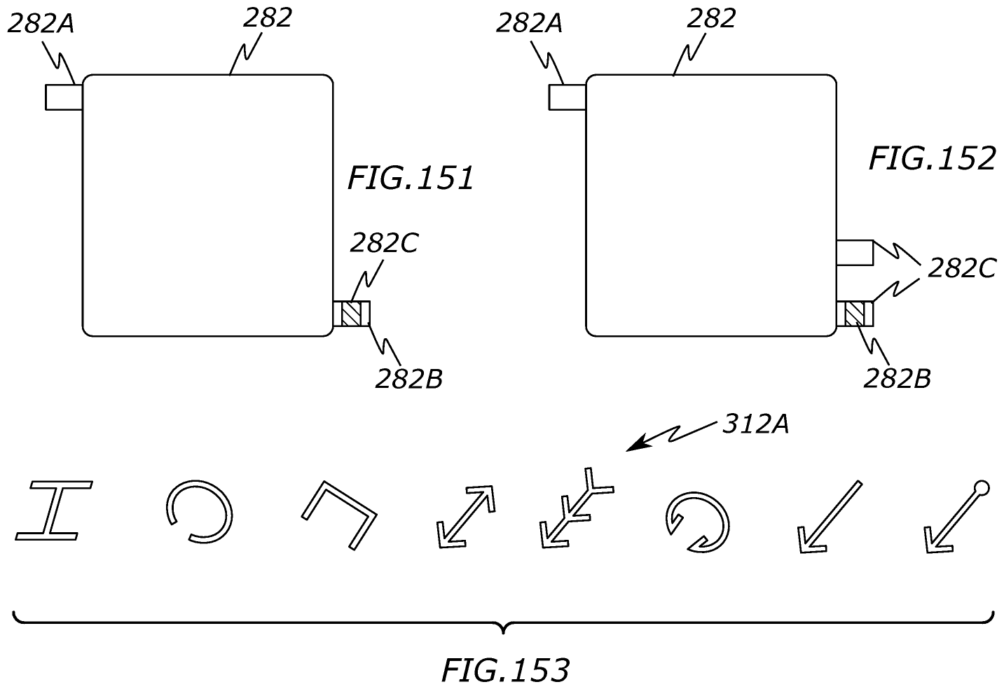

In another embodiment seen in FIG. 151, the reservoir 282 may include a filter 282C in an outlet conduit 282B so that only the desired constituents of the lymphatic fluid are drained into the reservoir 282.

In another embodiment, the reservoir 282 may be comprised of more than one outlet conduit 282B, at least one of which includes a filter 282C. This may allow both filtered and unfiltered fluid to be removed from the reservoir 282. It may be advantageous to withdraw fluid from the filtered outlet conduit and discard and then withdraw the remaining fluid from the reservoir 282 through the unfiltered outlet conduit. The second fluid removed may be re-introduced to the patient or discarded.

It would be appreciated by one skilled in the art that any of the above embodiments are not mutually exclusive of each other and can be combined in a variety of different combinations.

As discussed in the methods and embodiments below, a patient's lymphatic vessel can be accessed many times for drainage purposes over an extended period of time to manage a chronic condition such as heart failure. For simplicity, the thoracic duct 20 will be used as an example of a lymphatic structure; however, any lymphatic structure can be used without deviating from the present invention. It should be appreciated that any of the embodiments related to draining lymphatic fluid in this specification may be combined with any of the long term drainage embodiments discussed below.

In one embodiment, a marker device 312 may be placed within the patient in or near a lymphatic structure to allow its location to be repeatably identified to re-access the lymphatic vessel. The marker 312 may be identified using any visualization means known to one skilled in the art, including but not limited to ultrasound, fluoroscopy, Computed Tomography (CT), Magnetic Resonance Imaging (MRI), or X-Ray. The marker 312 may be comprised of one or more materials suitable for visualization using one or more imaging modalities including but not limited to steel, stainless steel, aluminum, chromium, barium, iodine, gadolinium, alloys or composite materials containing one or more of these materials, plastics, plastics with one or more radiopaque or fluorogenic additives such as barium, or plastics with one or more MRI contrast agents such as gadolinium.

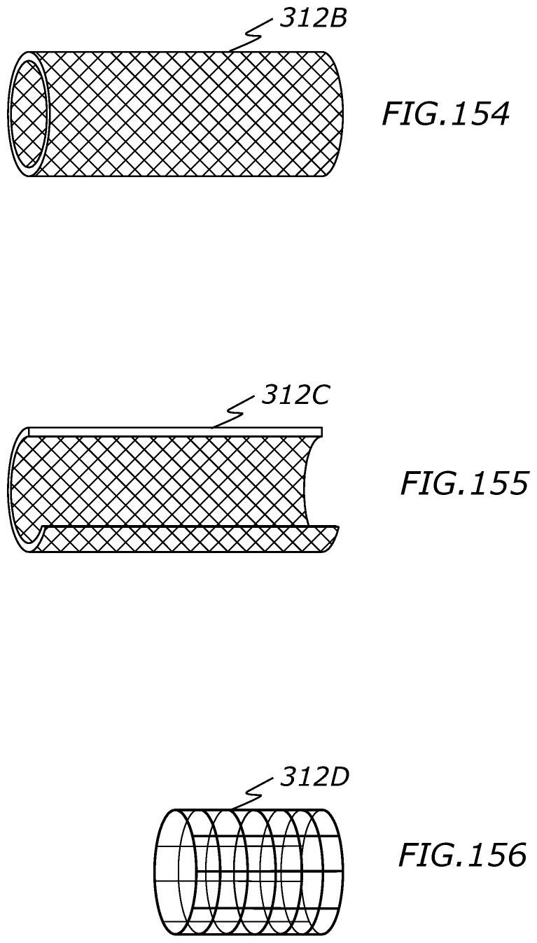

In one embodiment seen in FIG. 153, at least part of the marker 312 may comprise an anchor shape 312A, such as a clip, staple, t-tag, or barbed anchor. The marker 312 may also comprise a tubular stent shape 312B (e.g., mesh or laser cut tube), as seen in FIG. 154, a partial tubular structure 312C (e.g., a cross sectional “C” shape) as seen in FIG. 155, or a tubular frame 312D formed from a plurality of struts/rings creating the outline of a cylinder as seen in FIG. 156.

Alternately, the marker 312 may also be an adhesive patch or ink-based injection. The marker 312 may further be partially or fully comprised of a magnetic material to help with both visualization and detection of its magnetic fields via a sensor.

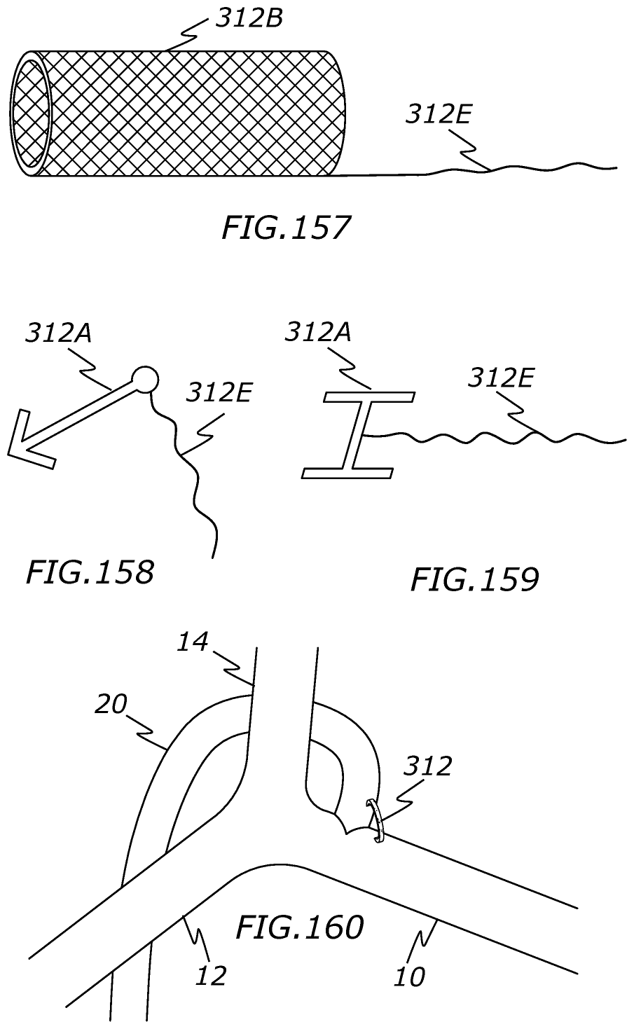

The marker 312 may further comprise a section of wire attached to it to help with visualization. For example, FIG. 157 illustrates a wire 312E attached to and extending away from a tubular stent shape 312B. In another example, FIG. 158 illustrates a wire 312E attached to and extending away from a barb anchor 312A. In another example, FIG. 159 illustrates a wire 312E attached to and extending away from a “I” shaped anchor 312A. The wire 312E of these embodiments may be partially or fully comprised of a magnetic material.

In another example, the marker 312 may contain a sensor to measure one or more physiologic parameters including but not limited to pressure, flow rate, temperature, pH, salinity, and water content. The sensor /may be battery-powered or inductively-powered by placing a coil above the marker. The sensor data may be transmitted wirelessly to a receiver exterior to the body to record and display the data.

In one embodiment, a marker may be placed in a lymphatic vessel. The marker may be placed in the cervical portion 20A or thoracic portion 20B of the thoracic duct 20 of a patient. The marker 312 may be placed within, adjacent to, or near the cisterna chyli of a patient. The marker may be placed adjacent to the terminal valve of a thoracic duct of a patient.

In another embodiment, the marker 312 may be placed in a venous vessel near a lymphatic vessel or nearby or adjacent to the terminal portion of the thoracic duct 20. In another embodiment, the marker 312 may be placed in the soft tissue near a lymphatic vessel. In another embodiment, the marker 312 may be placed through one or more walls of a lymphatic vessel. In another embodiment, the marker 312 may be placed through one or more walls of a venous vessel.

In another embodiment, the marker may be placed through one or more walls of a venous vessel and one or more walls of a lymphatic vessel. For example, FIG. 160 illustrates a marker 312 positioned through a wall of the thoracic duct 20 and a wall of the left subclavian vein 10.

In another embodiment, the marker 312 may be placed at least partially in a venous vessel and partially in a thoracic duct with a portion extending across the terminal valve of the thoracic duct.

In another embodiment, the marker 312 may be placed on the skin of a patient near a lymphatic vessel. In another embodiment, the marker 132 may be placed around the outside of a lymphatic vessel. The marker may partially or fully surround the exterior of the vessel.

In another embodiment, the marker 312 may be attached to a leaflet of a valve in a lymphatic vessel including but not limited to a valve in the thoracic duct 20 or a leaflet of the terminal valve 22 of the thoracic duct 20.

In another embodiment, the marker 312 may be partially or fully comprised of a magnetic material to allow identification by means of a magnetic probe such as a catheter with a magnetic distal end. In this respect, the catheter may magnetically attract the distal end of the catheter to the marker 312 when advanced to a location nearby.

A re-access and drainage system may include an implanted or indwelling catheter 116 and a re-access catheter 119 that is connected to the indwelling catheter 116 at one or more occasions.

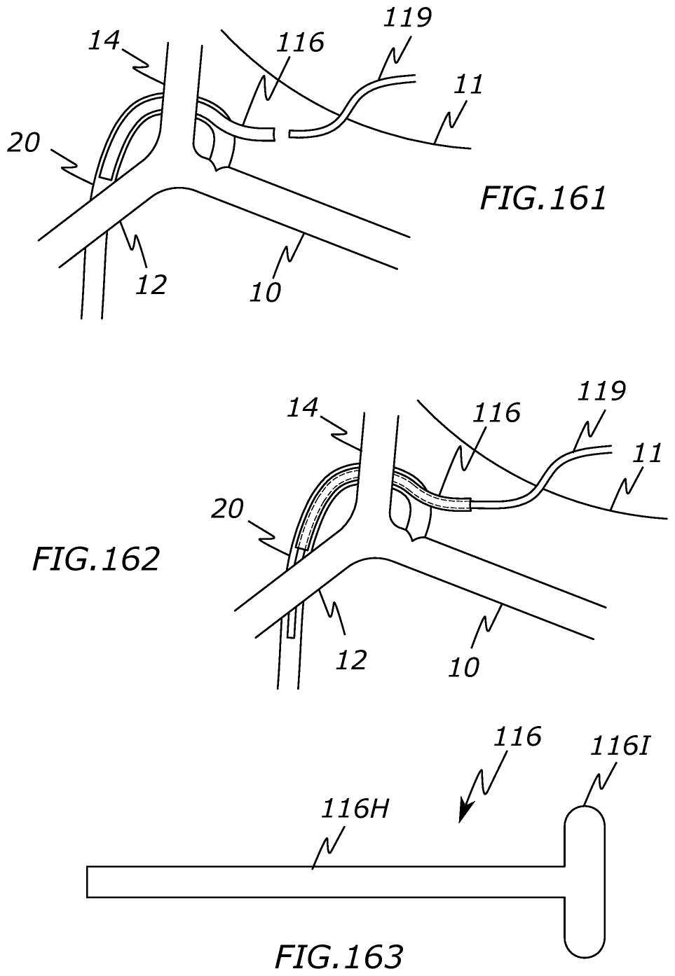

In one embodiment seen in FIGS. 161 and 162, a distal end of the indwelling access catheter 116 may be placed in a lymphatic vessel, such as a thoracic duct 20, as described previously. At a desired time for drainage, the proximal end of an access catheter 116 may be identified using tactile or visualization techniques such as ultrasound, and a drainage catheter 119 may be advanced into and through the access catheter into the lymphatic vessel to drain lymphatic fluid. In this respect, the distal end of the drainage catheter 119 is positioned distally beyond or out of the indwelling catheter 116. The access catheter 116 may have a proximal end in a venous vessel or in the tissue surrounding the lymphatic vessel and may be secured with hooks, barbs, suture, or staples. The access catheter 116 may have a valve, such as a duckbill valve, to prevent flow without the presence of a drainage catheter 119 positioned therethrough.

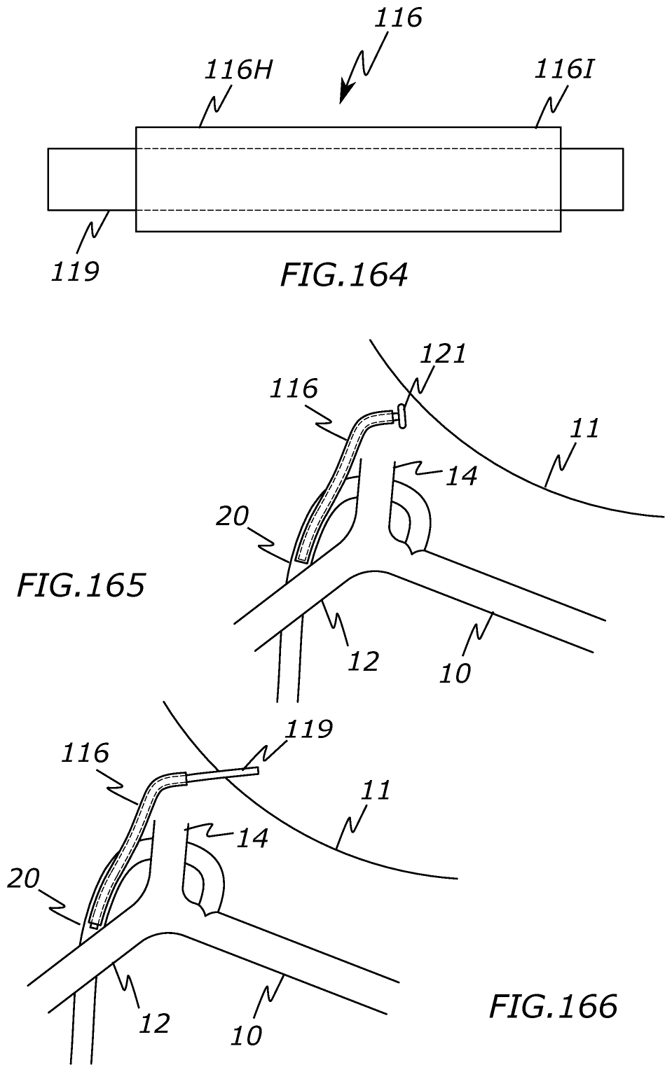

The access catheter 116 may be comprised of a soft material that radially collapses without a drainage catheter 119 inside to support the structure to prevent flow when drainage is not desired. For example, FIG. 163 illustrates a catheter 116 having a soft, collapsible body portion 116H and a radially-rigid proximal opening 116I that remains expanded without a drainage catheter 119 inside. FIG. 164 illustrates a drainage catheter 119 advanced through the access catheter 116, causing the body portion 116H to radially expand. Once the draining therapy is complete, the drainage catheter 119 may be removed, and the access catheter 116 may remain in place with its body portion 116H collapsed for another drainage therapy in the future.

In another embodiment seen in FIGS. 165 and 166, a re-access and drainage system may be comprised of an indwelling access catheter 116, a stylet 121, and a drainage catheter 119. The indwelling access catheter 116 may be placed with a distal end in a lymphatic vessel (e.g., thoracic duct 20) as described previously. The stylet 121 may be placed in the lumen of the access catheter 116 to occlude the lumen and prevent fluid flow when drainage is not desired. At a desired time for drainage, the stylet 121 may be removed, and the drainage catheter 119 may be advanced into and through the access catheter 116 into the lymphatic vessel to drain lymphatic fluid.

Alternatively, the stylet 121 may be inflatable to occlude the lumen of the indwelling access catheter 116 when inflated and permit flow when deflated so the stylet 121 can remain within the catheter 116 when the drainage catheter 119 is inserted. Alternatively, the indwelling access catheter 116 may include an integral balloon to inflate and occlude the lumen to prevent flow.

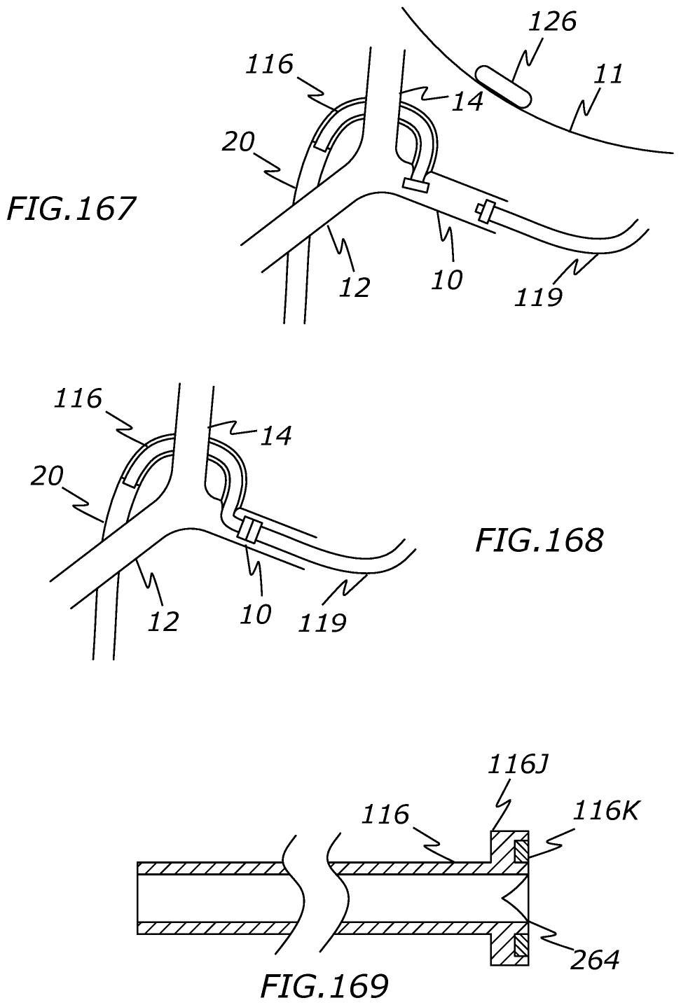

In another embodiment seen in FIGS. 167 and 168, a re-access and drainage system may be comprised of an indwelling access catheter 116 and a removable/replaceable drainage catheter 119 that is configured to be advanced through venous vessels. The indwelling drainage catheter 116 may have a distal end positioned in a lymphatic vessel (e.g., thoracic duct 20) and a proximal end in a venous vessel (e.g., left subclavian vein10) or in the tissue around the lymphatic vessel. The indwelling drainage catheter 116 may be anchored in the lymphatic vessel using attachment mechanism described elsewhere in this application. The indwelling drainage catheter 116 may have one or more valves to prevent flow from the lymphatic vessel when drainage is not desired. The proximal end of the indwelling drainage catheter 116 may be located by tactile or visualization means such as ultrasound via ultrasound transducer 126. The re-access drainage catheter 119 may be advanced through the skin 11 and the distal end may be advanced to the proximal end of the indwelling drainage catheter 116. Once the two ends are in contact, lymphatic fluid may be drained through both catheters to remove lymphatic fluid from the patient.

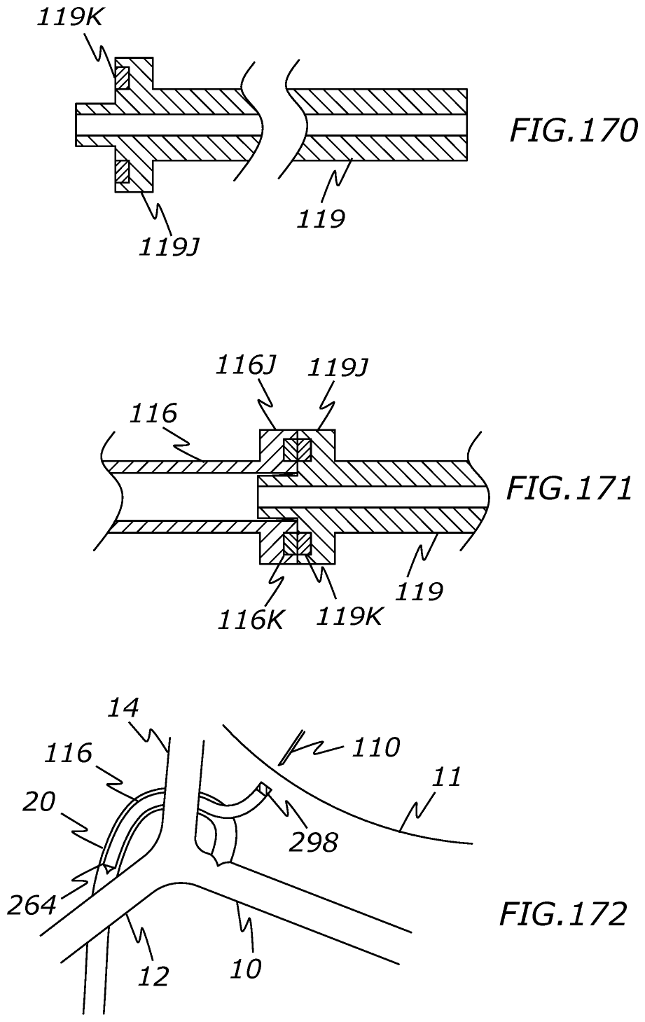

The two contact ends of the catheters 116, 119 may be configured to easily align and connect to each other. In the example of FIGS. 169-171, the ends of the catheters 116, 119 may include magnetic portions 116K, 119K that are configured to attract each other to cause the ends to align and come together. The two contact ends may further include flanges 116J and 119J that are disposed around the outer circumferences of the catheters and that help provide greater contact areas to connect together and optionally provide a location for the magnets 116K, 119K. The distal end of the re-access drainage catheter 119 may also be configured to open a valve 264, such as a duckbill valve, in the indwelling drainage catheter 116 when the two ends are in contact to permit flow. Alternately or additionally, the two contact ends may be threaded to aid with intra-procedural alignment. Additionally, the contact ends may be partially or fully comprised of radiopaque materials to provide intraoperative confirmation of successful contact.

In another embodiment seen in FIG. 172, the indwelling access catheter 116 may be placed with a distal end in a lymphatic vessel (e.g., thoracic duct 20) as described previously with a proximal end underneath the skin 11 of the patient. The proximal end, distal end, or both ends of the access catheter 116 may have one or more valves 264 or septa 298 to prevent flow when drainage is not desired.

This specification is also directed to several different aspects of drainage system configurations for acutely draining lymphatic fluids. It can be appreciated that the elements and steps of the examples below may be re-arranged in additional combinations with fewer, greater, or the same numbers of elements and steps. For simplicity, the thoracic duct will be used as an example of a lymphatic structure; however, any lymphatic structure may be used without deviating from the present invention.

It may be advantageous to provide a system and method to access and drain lymphatic fluid in the manner illustrated in the following examples.

In one example, a system and method for removing fluid from a lymphatic system in a patient may include a needle 110. A lymphatic structure may be imaged using ultrasound 126 and the needle 110 advanced into the lymphatic structure. Lymphatic fluid may be drained through the needle 110.

In one example, a system and method for removing fluid from a lymphatic system in a patient may include a needle 110, guidewire 117, snare catheter 127 (e.g., a catheter with one or more loops at its distal end that can be decreased in diameter when proximally withdrawn), and a drainage catheter 116. A lymphatic structure may be imaged using ultrasound and the needle 110 advanced into the lymphatic structure. A guidewire 117 may be inserted through the needle 110 and advanced antegrade into a venous vessel. A snare catheter 127 may be used to capture the guidewire 117 and a drainage catheter 116 may be advanced over the snare catheter 127 and guidewire 117 and into the lymphatic structure. Lymphatic fluid may be drained through the drainage catheter 116.

In one example, a system and method for removing fluid from a lymphatic system in a patient may include a catheter 116 and contrast media. The catheter 116 may be inserted into a vein in a patient and advanced toward the terminal end of the thoracic duct 20. Contrast may be injected near the terminal end of the thoracic duct 20 to identify its exact location using fluoroscopy. After identifying the thoracic duct 20, the catheter 116 may be advanced into the thoracic duct 20. Lymphatic fluid may be drained through the catheter 116.

In one example, a system and method for removing fluid from a lymphatic system in a patient may include a catheter 116 and ultrasound transducer 126. The catheter 116 may be inserted into a vein in a patient and advanced toward the desired lymphatic structure under ultrasound visualization. After identifying the terminal portion of the thoracic duct 20, the catheter 116 may be advanced into the desired lymphatic structure. Lymphatic fluid may be drained through the catheter 116.

In one example, a system and method for removing fluid from a lymphatic system in a patient may include a catheter 116 and contrast media. Contrast media may be injected into a lymphatic structure in a patient to opacify the lymphatic system. The catheter 116 may be inserted into a vein in a patient and advanced toward the opacified thoracic duct 20. The catheter 116 may be advanced into the thoracic duct 20, and lymphatic fluid may be drained through the catheter 116.

In one example, a system and method for removing fluid from a lymphatic system in a patient may include a catheter 116 and fluorescent media such as indocyanine green (ICG). The fluorescent media may be injected into a lymphatic structure in a patient. An incision near the desired lymphatic structure could be made to expose the lymphatic structure. Light of the wavelength to cause fluorescence of the media may be directed toward the surgical field using a system such as the SpyElite (Stryker) to allow direct visualization of the fluorescent material within the lymphatic structure. The catheter 116 may be surgically placed directly into the lymphatic structure, and lymphatic fluid may be drained through the catheter 116.

In one example, a system and method for removing fluid from a lymphatic system in a patient may include a needle 110, guidewire 117, and catheter 116. A lymphatic structure may be imaged using ultrasound and the needle 110 advanced into the lymphatic structure through a vein. The guidewire 117 may be inserted through the needle 110 into the lymphatic structure, and the needle 110 may be withdrawn. The catheter 116 may be advanced over the guidewire 117 into the lymphatic structure, and lymphatic fluid may be drained through the catheter 116.

In one example, a system and method for removing fluid from a lymphatic system in a patient may include a catheter and a pre-operative imaging study. A pre-operative imaging study such as MRI or CT may be performed to identify the location of a desired lymphatic structure to access. During the procedure, the pre-operative imaging study data may be overlayed onto the real-time fluoroscopic images. The catheter 116 and guidewire 117 may be inserted into a vein in the patient and advanced toward and into the desired lymphatic structure. Lymphatic fluid may be drained through the catheter 116.

It may be advantageous to provide a system and method to access and drain lymphatic fluid from a patient during multiple procedures, as the following examples illustrate.

In one example, a system and method for removing fluid from a lymphatic system from a patient during multiple procedures includes the use of two or more needles 110, a catheter 116, and subcutaneous port 244. A surgical cut-down procedure may be performed to access a desired lymphatic structure. The distal end of the catheter 116 may be placed into the desired lymphatic structure and attached to the lymphatic structure as described elsewhere in this application. The proximal end of the catheter 116 may be attached to the subcutaneous port 244. The subcutaneous port 244 may be implanted subcutaneously and secured to the surrounding tissue and the surgical site closed. A first needle 110 may access the port 244 through the skin and drain lymphatic fluid. The needle 110 may be removed from the port when the draining therapy is complete. When another drainage therapy session is required, a second needle 110 may be used to re-access the port and perform another drainage therapy.

In one example, a system and method for removing fluid from a lymphatic system in a patient during multiple procedures includes the use of two or more needles 110, a marker device 312, guidewire 117, catheter 116, and port 244. A lymphatic structure may be imaged using an ultrasound transducer 126 and the first needle 110 advanced into the lymphatic structure. A marker device 312 may be deployed in the lymphatic structure where needle access is desired. The guidewire 117 may be advanced through the needle 110 and the needle 110 may be removed. The catheter 116 may be advanced over the guidewire 117 and partially into the lymphatic structure (e.g., thoracic duct 20). The distal end of the catheter 116 may be coupled to the marker device 312 (e.g., via hooks, magnets or similar mechanisms) and the proximal end of the catheter 116 may be coupled to a port 244 and secured in the subcutaneous tissue. Lymphatic fluid may be removed by inserting the needle 110 into the port 244 and draining lymphatic fluid. The needle 110 may be removed when drainage therapy is complete. When another drainage therapy session is required, a second needle 110 may be used to re-access the port and perform another drainage therapy.

In one example, a system and method for removing fluid from a lymphatic system in a patient during multiple procedures includes a needle 110, guidewire 117, port 244, and two or more drainage catheters 116. A lymphatic structure may be imaged using an ultrasound transducer 126 and the needle 110 is advanced into the lymphatic structure. The guidewire 117 may be placed through the needle 110 and into the lymphatic structure. The proximal end of the guidewire 117 may be advanced through port 244 and the port secured in the subcutaneous tissue. A first drainage catheter 116 may be advanced through the port 244 and over the wire and into the lymphatic structure to access the lymphatic fluid. The lymphatic fluid may be drained through the catheter 116 and removed when drainage therapy is complete. When another drainage therapy session is required, a second drainage catheter 116 may be inserted through the port 244 and over the wire to re-access the lymphatic fluid and perform another drainage therapy.

In one example, a system and method for removing fluid from a lymphatic system in a patient during multiple procedures includes a catheter 116, guidewire 117, port 244, and two or more needles 110. A desired lymphatic structure may be identified using ultrasound (e.g., an ultrasound transducer 126) or fluoroscopy. The catheter 116 and guidewire 117 may be inserted into the vein of a patient and advanced into the desired lymphatic structure. The guidewire 117 may be removed and the distal end of the catheter 116 left in the desired lymphatic structure. The proximal end of the catheter 116 may be attached to the port 244, and the port 244 secured in the subcutaneous tissue. The needle 110 may be inserted into the port to access and drain lymphatic fluid and be removed when the drainage therapy is complete. When another drainage therapy session is required, a new needle 110 may be used to re-access the lymphatic fluid and perform another drainage therapy.

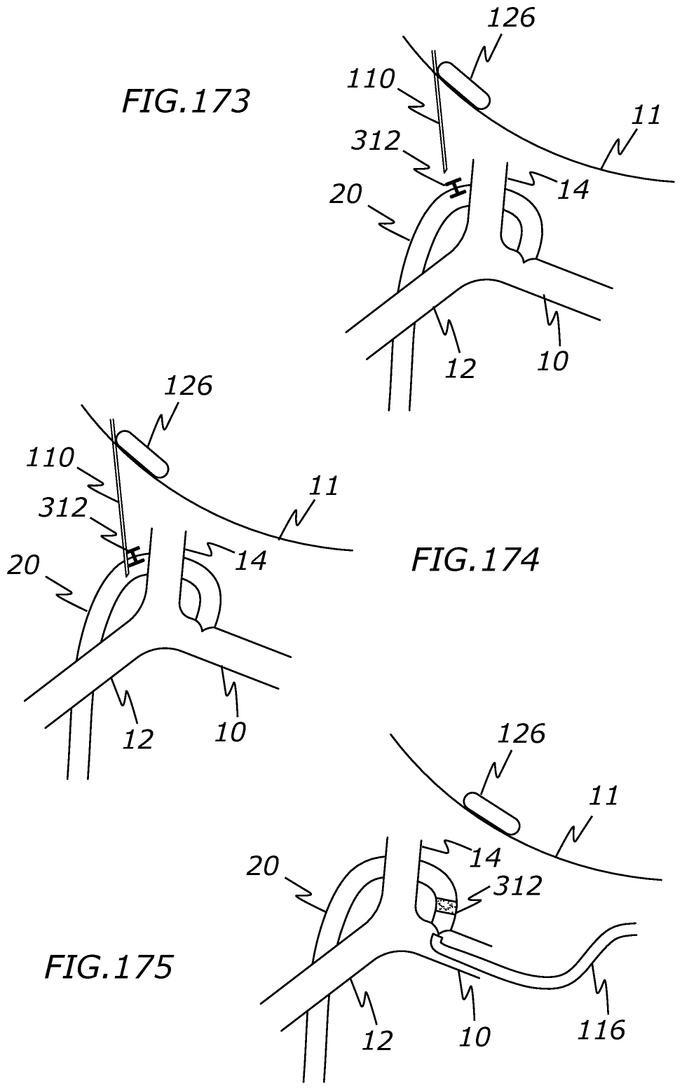

In one example seen in FIGS. 173 and 174, a system and method for removing fluid from a lymphatic system in a patient during multiple procedures includes two or more needles 110 and a marker device 312 (e.g., as discussed elsewhere in this specification). A desired lymphatic structure may be identified using ultrasound (e.g., via an ultrasound transducer 126) or fluoroscopy. A first needle 110 may be advanced into the desired lymphatic structure and a marker device 312 is delivered in or near the desired lymphatic structure. The needle 110 may be used to drain lymphatic fluid and be removed after the drainage therapy is complete without removing the marker device 312. When another drainage therapy session is required, the marker device 312 may be located to easily identify the desired lymphatic structure and a second needle 110 may be inserted and used to perform another drainage therapy, as seen in FIGS. 173 and 174.

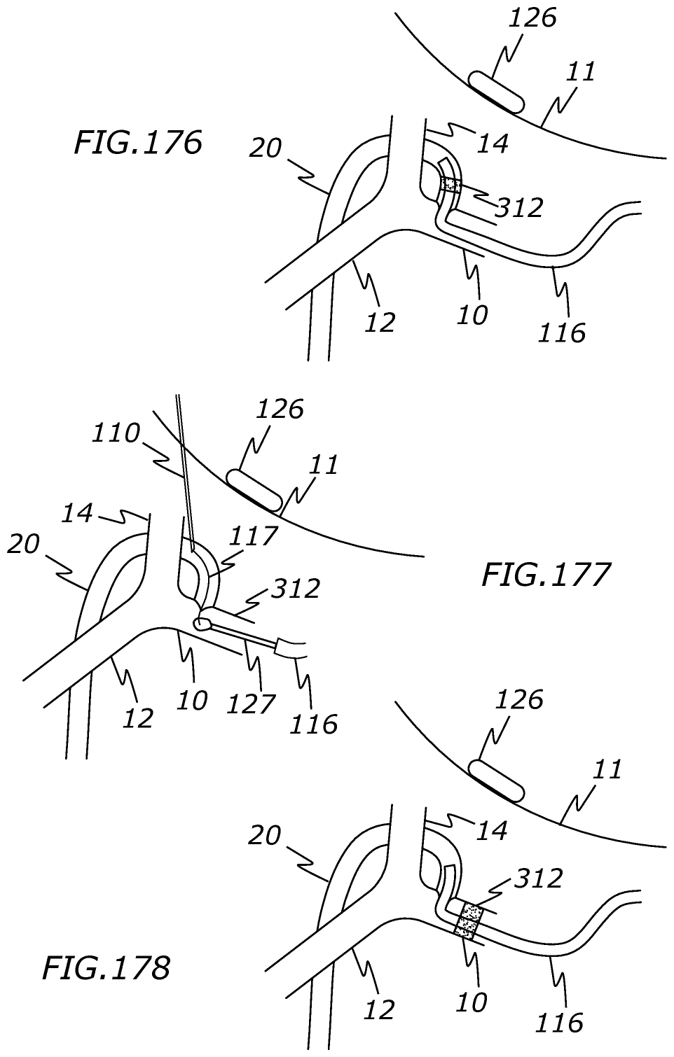

In one example seen in FIGS. 175 and 176, a system and method for removing fluid from a lymphatic system in a patient during multiple procedures includes a needle 110, marker device 312, and two or more drainage catheters 116. A desired lymphatic structure may be identified using ultrasound (e.g., via an ultrasound transducer 126) or fluoroscopy. The needle 110 may be advanced into the desired lymphatic structure and the marker placed in or near the desired lymphatic vessel (e.g., thoracic duct 20). A first drainage catheter 116 may be inserted into the vein of a patient (e.g., left subclavian vein 10) and advanced toward the marker device 312 and into the desired lymphatic structure using ultrasound (e.g., via an ultrasound transducer 126) or fluoroscopic guidance. Lymphatic fluid may be drained through the catheter 116. The catheter 116 may be removed after the drainage therapy is complete. When another drainage therapy session is required, a second drainage catheter 116 may be inserted and advanced toward the marker device 312 and into the desired lymphatic structure to perform another drainage therapy.

In one example, a system and method for removing fluid from a lymphatic system in a patient during multiple procedures includes a needle 110, guidewire 117, a snare catheter 127, a stent marker 312, a delivery and drainage catheter 116, and re-access and drainage catheter 116. A desired lymphatic structure may be identified using ultrasound (e.g., via an ultrasound transducer 126) or fluoroscopy. The needle 110 may be advanced into the desired lymphatic structure and a guidewire 117 advanced through the needle antegrade into the venous system. The snare catheter 127 may snare the guidewire 117, and the delivery and drainage catheter 116 may be advanced over the snare catheter 127 and guidewire 117 and into the desired lymphatic structure. The delivery and drainage catheter 116 may deploy a marker device 312 (e.g., a stent marker 312B) in the desired lymphatic structure and drain lymphatic fluid. When the drainage therapy is complete, the delivery and drainage catheter 116 may be removed and leave the stent marker in the lymphatic structure. When another drainage therapy session is required, a re-access and drainage catheter 116 may be inserted and advanced toward the marker device 312 using ultrasound guidance and into the desired lymphatic structure to perform another drainage therapy.

In one example seen in FIGS. 177 and 178, a system and method for removing fluid from a lymphatic system in a patient during multiple procedures includes a needle 110, a guidewire 117, a snare catheter 127, marker device 312, a delivery and drainage catheter 116, and a re-access and drainage catheter 116. A desired lymphatic structure (e.g., thoracic duct 20) may be identified using ultrasound (e.g., via an ultrasound transducer 126) or fluoroscopy. The needle 110 may be advanced into the desired lymphatic structure and a guidewire 117 advanced through the needle 110 antegrade into the venous system. As seen in FIG. 177, a distal loop on the snare catheter 127 may snare the guidewire 117, which then allows the delivery and drainage catheter 116 to be advanced over the snare catheter 127 and guidewire 117, into the thoracic duct 20. The delivery and drainage catheter 116 may drain lymphatic fluid and be partially removed following completion of the drainage therapy. Prior to removal from the body, the delivery and drainage catheter 116 may deploy a marker device 312 (e.g., a stent marker 312B) in a venous structure near the junction with the target lymphatic structure for ease of identification in the future. The delivery and drainage catheter 116 may then be fully removed, leaving the stent marker in place. When another drainage therapy session is required, a re-access and drainage catheter 116 may be inserted, as seen in FIG. 178, and advanced toward the marker device 312 using ultrasound guidance and into the desired lymphatic structure to perform another drainage therapy.

In one example, a system and method for removing fluid from a lymphatic system in a patient during multiple procedures includes a needle 110, a snare catheter 127, a stent marker 312 with attached magnetic wire 312E, and a delivery and drainage catheter 116. A desired lymphatic structure may be identified using ultrasound (e.g., via an ultrasound transducer 126) or fluoroscopy. The needle 110 may be advanced into the desired lymphatic structure and a guidewire 117 is advanced through the needle antegrade into the venous system. The snare catheter 127 may snare the guidewire 117, and the delivery and drainage catheter 116 may be advanced over the snare catheter 127 and guidewire 117, into the desired lymphatic structure. The delivery and drainage catheter 116 may deploy a marker device 312 (e.g., a stent marker 312B) with attached magnetic wire 312E in the desired lymphatic structure and drain lymphatic fluid. When the drainage therapy is complete, the delivery and drainage catheter 116 may be removed and the marker device 312 left in the lymphatic structure with the attached magnetic wire 312E in a venous vessel. When another drainage therapy session is required, a re-access and drainage catheter 116 may be inserted into the venous vessel and coupled with the magnetic wire 312E (e.g., via snare catheter 127 or magnetic distal tip). The re-access and drainage catheter 116 may then be advanced into the desired lymphatic vessel to perform another drainage therapy.

In one example, a system and method for removing fluid from a lymphatic system in a patient during multiple procedures includes a needle 110, a snare catheter 127, a marker device 312, a delivery and drainage catheter 116, and a re-access and drainage catheter 116. A desired lymphatic structure may be identified using ultrasound (e.g., via an ultrasound transducer 126) or fluoroscopy. The needle 110 may be advanced into the desired lymphatic structure and a guidewire 117 advanced through the needle 110 antegrade into the venous system. The snare catheter 127 may snare the guidewire 117, and the delivery and drainage catheter 116 may be advanced over the snare catheter 127 and guidewire 117, into the desired lymphatic structure. The delivery and drainage catheter 116 may drain lymphatic fluid and be partially removed following completion of the drainage therapy. Prior to removal from the body, the delivery and drainage catheter may deploy a marker device 312 (e.g., stent marker 312B) across the junction of a venous vessel and the target lymphatic structure for ease of identification in the future. The delivery and drainage catheter 116 may then be fully removed and the marker device 312 left in place. When another drainage therapy session is required, a re-access and drainage catheter 116 may be inserted and advanced toward the marker device 312 using ultrasound guidance and into the desired lymphatic structure to perform another drainage therapy.

In one example, a system and method for removing fluid from a lymphatic system in a patient during multiple procedures includes an indwelling access catheter 116 and a drainage catheter 119. The indwelling access catheter 116 may be placed with a distal end in a lymphatic vessel (e.g., the thoracic duct 20) as described elsewhere in this application. At a desired time for drainage, the proximal end of an access catheter may be identified using tactile or visualization means such as ultrasound, and a drainage catheter 119 may be advanced into and through the access catheter 116 into the lymphatic vessel to drain lymphatic fluid. Once the draining therapy is complete, the drainage catheter may be removed, and the access catheter 116 may remain in place for another drainage therapy in the future.

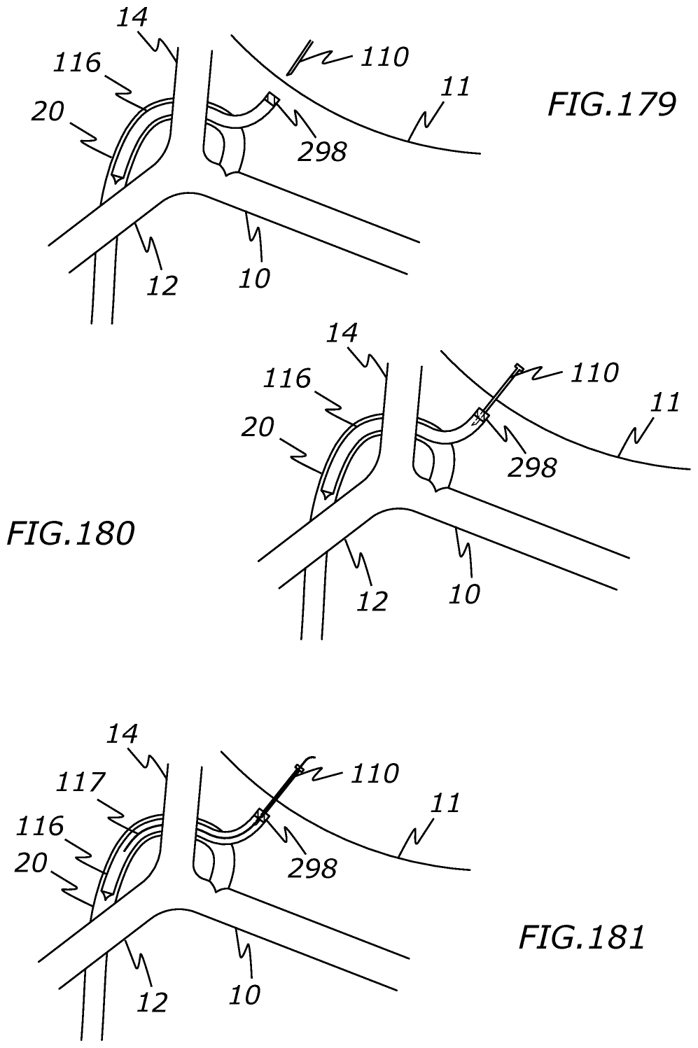

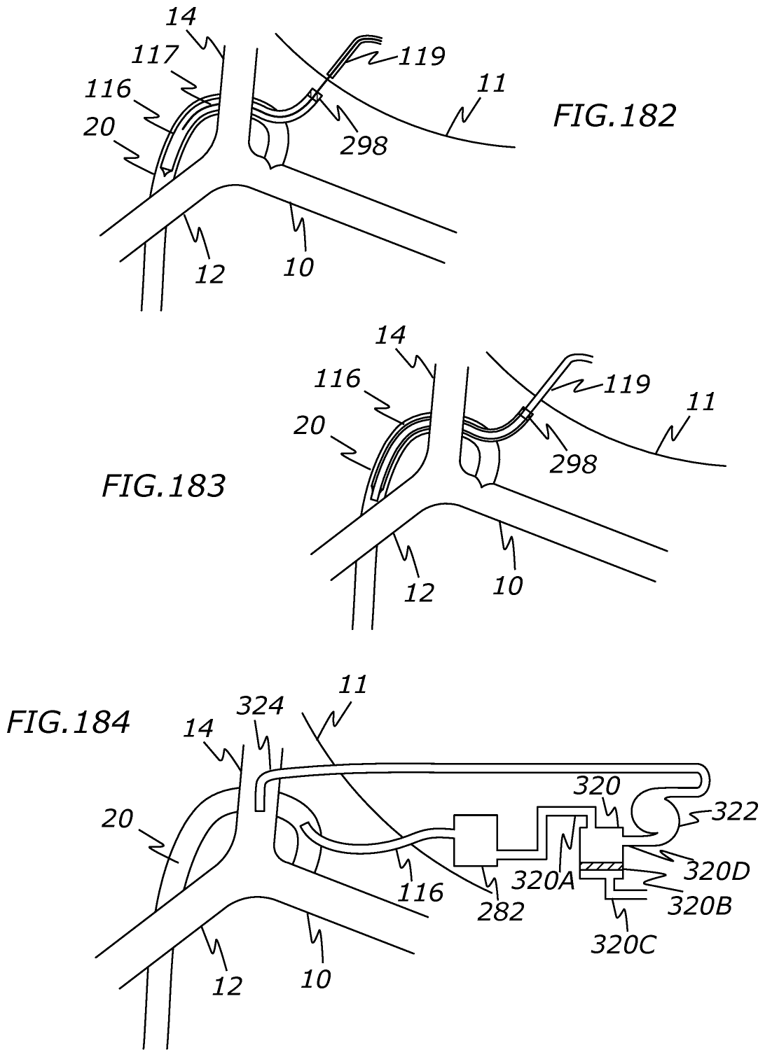

In another embodiment, a re-access system may be comprised of an indwelling access catheter 116, a needle 110, a guidewire 117, and a drainage catheter 119. The indwelling access catheter 116 may be placed with a distal end in a lymphatic vessel (e.g., thoracic duct 20) as described previously with a proximal end with septum 298 underneath the skin 11 of the patient. At a desired time for drainage, the proximal end (e.g., septum 298) of the access catheter 116 may be identified, as seen in FIG. 179. The needle 110 may be advanced into the septum 298 of the access catheter 116 as seen in FIG. 180, and fluid may be aspirated to confirm it is in the correct location by means described elsewhere in this application. As seen in FIG. 181, once the needle 110 is confirmed to be in the access catheter 116, a guidewire 117 may be placed through the needle 110 and into the access catheter 116, and the needle 110 may be removed. A drainage catheter 119 may then be inserted into the access catheter 116 over the guidewire and advanced into the lymphatic vessel to drain fluid as seen in FIGS. 182 and 183. Once the draining therapy is complete, the drainage catheter 119 may be removed, and the access catheter 116 may remain in place for another drainage therapy in the future.

It may be advantageous to provide a system and method to access and drain lymphatic fluid with an intermediate reservoir to allow lymphatic fluid drainage and emptying.

In one example, a system and method for removing fluid from a lymphatic system in a patient may include a needle 110, a guidewire 117, a catheter 116, a reservoir 282 with an outlet conduit 282B including a valve 264. A desired lymphatic structure may be identified using ultrasound or fluoroscopy. The needle 110 may be advanced into the desired lymphatic structure and a guidewire 117 is advanced through the needle 117. The needle 117 may be removed, and the catheter 116 may be advanced over the guidewire 117. The distal end of the catheter 116 may be left in the desired lymphatic structure, and the proximal end may be attached to a reservoir 282 outside of the patient. Lymphatic fluid may continuously drain into the reservoir 282. The patient may empty the reservoir by opening the valve 264 in the outlet conduit 282B as needed to allow additional lymphatic fluid to be drained.

In one example, a system and method for removing fluid from a lymphatic system in a patient may include a catheter 116 and a reservoir 282 with an outlet conduit 282B including a valve 264. A desired lymphatic structure may be identified using ultrasound or fluoroscopy. The catheter 116 may be introduced into a venous vessel and advanced into the desired lymphatic structure, and the distal end of the catheter 116 may be left in the lymphatic structure. The proximal end of the catheter 116 may be attached to a reservoir 282 outside of the patient and lymphatic fluid may continuously drain into the reservoir 282. The patient may empty the reservoir 282 by opening the outlet valve 264 as needed to allow additional lymphatic fluid to be drained.

In one example, a system and method for removing fluid from a lymphatic system in a patient may include a needle 110, a guidewire 117, a catheter 116, a reservoir 282 with an inlet conduit 282A and an outlet conduit 282B that both include a valve 264. A desired lymphatic structure may be identified using ultrasound or fluoroscopy. The needle 110 may be advanced into the desired lymphatic structure and a guidewire 117 advanced through the needle 110. The needle 110 may be removed, and the catheter 116 may be advanced over the guidewire 117. The distal end of the catheter 116 may be left in the desired lymphatic structure, and the proximal end of the catheter 116 may be attached to a reservoir 282 outside of the patient. The patient may open the valve 262 of the inlet conduit 282A in the reservoir 282 to allow lymphatic fluid to drain into the reservoir 282. The patient may close the valve 264 of the inlet conduit 282A when drainage is not needed. The patient may open the valve 264 of the outlet conduit 282B to empty the reservoir 282 as needed to allow additional lymphatic fluid to be drained.

In one example, a system and method for removing fluid from a lymphatic system in a patient may include a catheter 116 and a reservoir 282 with an inlet conduit 282A and an outlet conduit 282B that both include a valve 264. A desired lymphatic structure may be identified using ultrasound or fluoroscopy. The catheter 116 may be introduced into a venous structure and advanced into the desired lymphatic structure, and the distal end of the catheter 116 may be left there. The proximal end of the catheter 116 may be attached to a reservoir 282 outside of the patient. The patient may open valve 262 of the inlet conduit 282A in the reservoir 282 to allow lymphatic fluid to drain into the reservoir 282. The patient may close the valve 264 of the inlet conduit 282A when drainage is not needed. The patient may open the valve 264 of the outlet conduit 282B to empty the reservoir 282 as needed to allow additional lymphatic fluid to be drained.

In one example, a system and method for removing fluid from a lymphatic system in a patient may include a needle 110, a guidewire 117, a catheter 116, a manifold, and a syringe 310 with position lock. A desired lymphatic structure may be identified using ultrasound or fluoroscopy. The needle 110 may be advanced into the desired lymphatic structure and a guidewire 117 advanced through the needle 110. The needle 110 may be removed and the catheter 116 may be advanced over the guidewire 117. The distal end of the catheter 116 may be left in the desired lymphatic structure, and the proximal end of the catheter 116 may be attached to the manifold and syringe 310 outside of the patient. The manifold may be comprised of a hollow block with an array of valves and connections to control the flow in and out. For example, the manifold may have the catheter 116 attached to the inlet with a one-way valve into the manifold, a syringe attached to an outlet with a valve to control flow in and out, and a disposal tube attached to a second outlet with a valve to control flow in and out. The syringe plunger may be pulled back to a desired level and locked in place to generate vacuum to remove lymphatic fluid through the drainage catheter and the lymphatic fluid may drain into the syringe. When the syringe is full, the valve in the manifold to the disposal tube may be opened and the syringe plunger may be unlocked and pushed forward to expel the fluid from the syringe and into the disposal tube. The disposal tube valve may then be closed, and the syringe plunger may be pulled back to initiate another drainage therapy.

It may be advantageous to monitor a physiologic signal in conjunction with the aforementioned systems and methods to access and drain lymphatic fluid to inform a clinical action or decision. Examples of such monitoring and sensor usage are described below.

In one example, a system and method for monitoring a physiologic signal may include a sensor 228 configured to sense pressure and a wireless transceiver assembly 188. The pressure sensor 228 may be placed in a lymphatic structure. If the pressure exceeds a threshold, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

In one example, a system and method for monitoring a physiologic signal may include a sensor 228 configured to sense pressure and a wireless transceiver assembly 188. The pressure sensor 228 may be placed in a venous structure. If the pressure exceeds a threshold, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

In one example, a system and method for monitoring a physiologic signal may include two sensors 228 configured to sense pressure and a wireless transceiver assembly 188. One pressure sensor 228 may be placed in a lymphatic structure and the other pressure sensor 228 may be placed in a venous structure. If the pressure difference between the lymphatic structure and venous structure (P_{lymphatic}−Pᵥₑₙₒᵤₛ) falls below a threshold such as 0 mmHg, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

In one example, a system and method for monitoring a physiologic signal may include a sensor 228 configured to sense pressure, a sensor 228 configured to sense flow, and a wireless transceiver assembly 188. The pressure sensor may be placed in a lymphatic structure. If the pressure exceeds a threshold, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods. The flow rate of the lymphatic fluid may be measured by the flow sensor 228 and provided to the patient or provider via the communication device in addition to the pressure data. These data sets may be used to optimize the therapy.

In one example, a system and method for monitoring a physiologic signal may include a sensor 228 configured to sense pressure and a wireless transceiver assembly 188. The pressure sensor may be placed in the subcutaneous space to measure the interstitial fluid pressure, as seen in U.S. Pub. No. 20180271371A1 which is herein incorporated by reference. If the pressure exceeds a threshold such as 0 mmHg, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

In one example, a system and method for monitoring a physiologic signal may include a sensor 228 configured to sense congestion (e.g., the ReDS lung fluid sensor system from Sensible Medical Innovations) and a wireless transceiver assembly 188. The congestion sensor may be placed external to the patient to measure the amount of water in the lungs of a patient, the amount of distention of the neck veins or other sign of congestion. If congestion is sensed, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

In one example, a system and method for monitoring a physiologic signal may include a wearable edema sensor 228 and a wireless transceiver assembly 188. The edema sensor may be placed on the outside of the body to detect edema in the toes, feet, ankles, legs, abdomen, neck, or arms. If edema is sensed, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

In one example, a system and method for monitoring a physiologic signal may include a pulmonary artery pressure sensor (e.g., a Cardiomems HF System from Abbott) and a wireless transceiver assembly 188. If the pressure exceeds a threshold, the wireless transceiver assembly 188 may transmit data or a signal that triggers a notification to the patient or physician to perform a drainage therapy (e.g., signaling on a separate device, such as a pressure monitor display). The lymphatic fluid may be drained using any of the aforementioned systems and methods.

It may be advantageous to include a controller 283 in conjunction with the aforementioned systems and methods to access and drain lymphatic fluid and sense physiologic signals to automatically initiate drainage therapy. The controller 283 may at least include a processor, memory, and software executable by the processor. The software may include algorithms that include 1) measuring, receiving, and storing sensor data, 2) actuating devices (e.g., valves in a drainage system), and/or 3) generating notifications to a user and/or medical staff locally or remotely.

In one example, a system and method to control lymphatic fluid drainage may include a drainage catheter 116 with a distal end of its lumen in a lymphatic structure, a reservoir 282 with an inlet conduit 282A including a valve 264, a sensor 228, and a controller. The sensor 228 may be configured to measure thoracic duct pressure and provide a signal to the controller. If the pressure exceeds a first threshold, the software executed by the controller may open the valve 264 at the inlet conduit 282A in a reservoir 282 connected to a drainage catheter to drain lymphatic fluid. When the pressure falls below a second threshold, the controller may close the valve 264 at the inlet conduit 282A to terminate the drainage therapy.

In one example, a system and method for control lymphatic fluid drainage may include a drainage catheter 116 with a distal end of its lumen in a lymphatic structure, a reservoir 282 with an inlet conduit 282A including a valve 264, a reservoir fill sensor, a sensor 228, and a controller. The sensor 228 may measure thoracic duct pressure and provide a signal to the controller (e.g., either a wired signal or a wireless data packet). If the pressure exceeds a first threshold, the controller may open the valve 264 of the inlet conduit 282A in a reservoir 282 connected to a drainage catheter 116b to drain lymphatic fluid. When the pressure falls below a second threshold, the controller may close the valve 264 of the inlet conduit 282A to terminate the drainage therapy. The reservoir fill sensor may also communicate with the controller, and the controller may close the valve 264 of the inlet conduit 282A if the reservoir fill sensor exceeds a threshold. The patient may empty or replace the reservoir to continue drainage.

In one example, a system and method for controlling lymphatic fluid drainage may include a drainage catheter 116 with the distal end of a lumen in a lymphatic structure, a reservoir 282 with an inlet conduit 282A including a valve 264, a reservoir fill sensor, a pressure sensor 228, a flow sensor 228, a controller, and a pump. The sensor may measure thoracic duct pressure and provide a signal to the controller. If the pressure exceeds a first threshold, the controller may open the valve 264 of the inlet conduit 282A in a reservoir 282 connected to a drainage catheter 116 to drain lymphatic fluid. The controller may be programmed with a target flow rate of fluid through the drainage catheter 116 as measured by a flow sensor 228. The controller may activate the pump (connected to the catheter 116 or reservoir 282) to achieve a desired flow rate or adjust the pressure generated by the pump to achieve a desired flow rate. When the pressure in the lymphatic structure falls below a second threshold, the controller may close the valve 264 of the inlet conduit 282A to terminate the drainage therapy. The reservoir fill sensor may also communicate with the controller, and the controller may close the valve 264 of the inlet conduit 282A if the reservoir fill sensor meets or exceeds a threshold. The patient may empty or replace the reservoir 282 to continue drainage.

In one example, a system and method for control lymphatic fluid drainage may include a shunt with a valve 264, a sensor 228, and a controller. The sensor 228 may measure thoracic duct pressure and provide a signal to the controller. If the pressure exceeds a first threshold, the controller may open the valve 264 in the shunt to drain lymphatic fluid.

It may be advantageous to include a filter 236 in the fluid removal pathway with any of the above drainage systems to selectively remove the desired constituents from the lymphatic fluid. The filter 236 may be included in a lumen of a needle 110, in a lumen of a drainage catheter 116, an inlet of a port 244, or in a separate connector placed in the fluid path.

It may be advantageous to include a filter in the reservoir 282 with any of the above drainage systems to selectively filter some or all of the lymphatic fluid. The filter 236 may be included in an inlet conduit of the reservoir or an outlet conduit of the reservoir.

It may be advantageous to include a suction source in the fluid removal pathway with any of the above drainage systems to enhance the fluid removal flow rate by increasing the pressure gradient driving flow from a lymphatic structure. The suction source may be a suction pump, a powered suction source such as an electronically-operated suction system, or a non-powered suction source such as a syringe or manually-operated suction system.

In one example, a system may be comprised of one or more of a needle, a guidewire, a port, a cleaning stylet, a drainage cannula, a dilator, an introducer, a syringe, a vacuum bag, a pump, a pressure regulator, a drainage collection canister, a pressure sensor, a flow sensor, an impedance sensor, a weight sensor, a stopcock, a manifold, a controller, a communications device, a microchip, a suture.

In one example, a method of draining fluid from a lymphatic system may include measuring one or more physiologic signals of a patient, setting a target value for the one or more signals with a controller, initiating drainage using a drainage system, continuing drainage until the target value is achieved, discontinuing drainage.

In one example, a method of draining fluid from a lymphatic system may include measuring the pressure in the thoracic duct with a sensor, setting a pressure target with a controller, initiating drainage using a drainage system, continuing drainage until the pressure target is achieved, discontinuing drainage.

In one example, a method of draining fluid from a lymphatic system may include measuring the pressure in a vein with a sensor, setting a pressure target with a controller, initiating drainage using a drainage system, continuing drainage until the pressure target is achieved, discontinuing drainage.

In one example, a method of draining fluid from a lymphatic system may include measuring the pressure in the thoracic duct with a sensor, initiating drainage using a drainage system, measuring the flow rate of the draining fluid, setting a desired flow rate threshold or pressure threshold, continuing drainage until the target is achieved, discontinuing drainage.

In one example, a method of draining fluid from a lymphatic system may include measuring the pressure in the thoracic duct with a sensor, initiating drainage using a drainage system, measuring the flow rate of the draining fluid and total volume of fluid removed, setting a target volume of fluid to be removed, continuing drainage until the target is achieved, discontinuing drainage.

In one example, a method of draining fluid from a lymphatic system may include measuring the pressure in the thoracic duct with a sensor, measuring the pressure in a vein with a second sensor, calculating the difference between the pressures, setting a target difference with a controller, initiating drainage using a drainage system, continuing drainage until the target difference is achieved, discontinuing drainage.

Alternatively, with any of the aforementioned systems, methods, and devices that access a lymphatic system or venous system of a patient for fluid removal, it is contemplated that the same fluid removal lumen may be used to deliver fluid into a lymphatic system or venous system of a patient for a therapeutic effect. The composition of this fluid may be optimized to replace any lost electrolytes, leukocytes, proteins, white blood cells, or other constituent of lymphatic fluid. Alternatively, this fluid may contain a therapeutic agent to address a disease state or condition of the lymphatic, circulatory, immune, or other body system. The therapeutic agent may include a chemotherapy agent, a radioactive agent, a pharmaceutical agent, a drug, an anti-platelet agent, an antibiotic agent, an immunological agent or a diuretic agent.

The following embodiments are directed to various aspects of managing fluid removed from the lymphatic system of a patient.

In one embodiment, lymphatic fluid is removed from a patient in a manner described elsewhere in this specification and discarded.

In another embodiment, lymphatic fluid is removed from a lymphatic vessel of a patient in a manner described elsewhere in this specification and returned to the body of the patient. The fluid may be returned into a lymphatic vessel, venous vessel, or other appropriate region of the body such as the peritoneal cavity, the digestive tract, or urinary tract.

In another embodiment, lymphatic fluid is subjected to filtration prior to being removed from a patient and the removed fluid that passes through the filter is removed and is discarded. The remaining fluid remains in the body of the patient.

In another embodiment, fluid is removed from a patient in any manner described elsewhere in this specification and filtered external to the patient. The fluid that passes through the filter is discarded and the remaining fluid is returned to the patient. The fluid to be returned may be re-introduced into a lymphatic vessel, venous vessel, or other appropriate region of the body such as the peritoneal cavity or the digestive tract to be reabsorbed. The fluid to be returned may be pumped back into the patient using a pump. As seen in the example of FIG. 184, a distal end of a drainage catheter 116 is placed in a lymphatic structure and connected to an input port 320A of a filter device 320. The filter device 320 may include a filter media 320B that prevents larger, beneficial particles from passing through so that they can be recirculated out the recirculation port 320D while mostly fluid alone (e.g., water) can be removed via discard outlet 320C. The recirculation port 320D may be connected to a pump 322 to help maintain a desired pressure throughout the system, and the pump 322 can be further connected to a return catheter 324 placed in a venous or lymphatic location in the patient.

In another embodiment, fluid is removed from a patient into a reservoir. After a sufficient amount of volume has been removed (e.g. 1L, 2L, or 3L), at least a portion of the fluid is filtered and the fluid that passes through the filter is discarded and the remaining fluid is returned to the patient. Alternatively, the fluid may be removed for a desired amount of time prior to filtering and returning the desired fluid to the patient (e.g. drain for 6 hours). The amount of fluid to be returned may be determined based on the physiologic response of the patient to the removal of lymphatic fluid as measured by at least one physiologic parameter including but not limited to blood pressure, white blood cell count, central venous pressure, thoracic duct pressure, edema, heart rate, or other signal discussed elsewhere in this application.

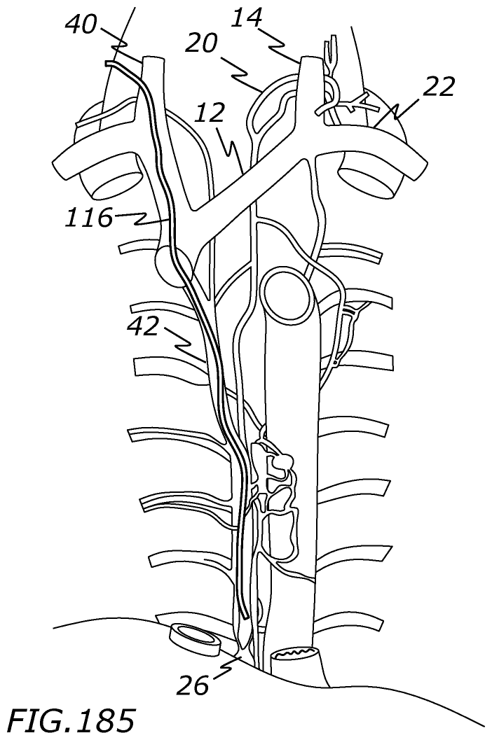

The following embodiments are directed to various aspects of example shunts and their use within a patient. Turning first to FIG. 185, this figure illustrates a procedure for accessing the lymphatic system via the venous system. The right internal jugular vein 40 of the patient is accessed by standard percutaneous methods known to one skilled in the art using standard devices including but not limited to a needle 110, a guidewire117, and/or catheter 116. After accessing the right internal jugular vein 40, a guidewire 117 and/or catheter 116 may be advanced into the Azygos vein 42 and moved distally toward the patients' abdomen until it is within proximity of a lymphatic structure such as the thoracic duct 20 or cisterna chyli 26.

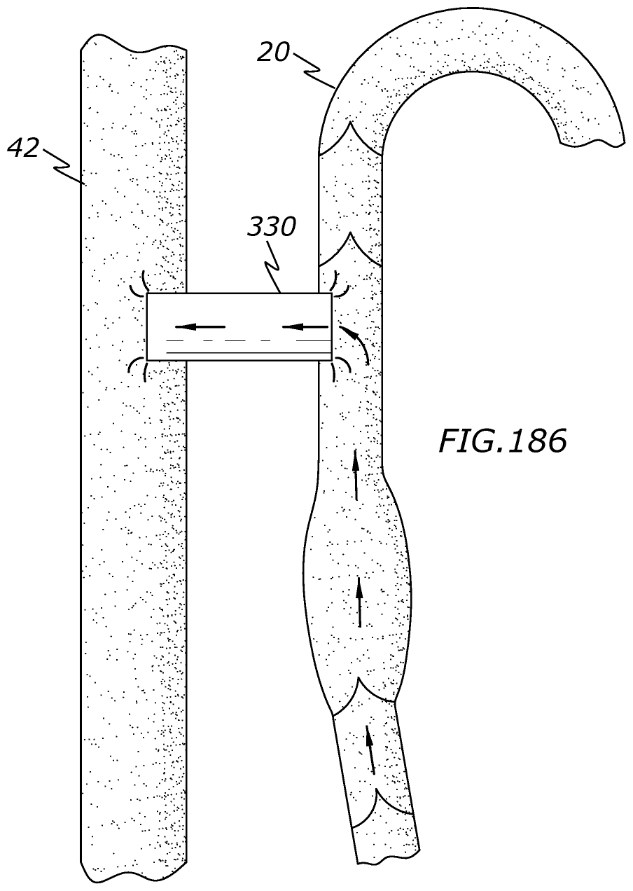

The lymphatic structure(s) may be identified by any method known to one skilled in the art including but not limited to intranodal lymphangiography, MRI, CT, and Ultrasound. Once the adjacent lymphatic structure is identified and confirmed to be in close proximity, the guidewire 117 and/or catheter 116 may cross into the lumen of the lymphatic structure to provide access for fluid drainage. The crossing into the lymphatic structure may be performed by a needle 110, sharp guidewire 117, Radio-Frequency guidewire, or other means known to one skilled in the art. Once both venous and lymphatic vessel lumens are accessed, a shunt 330 may be deployed to establishing a fluid path between the venous vessel (Azygos vein 42) and lymphatic structure (e.g., thoracic duct 20) as seen in FIG. 186.

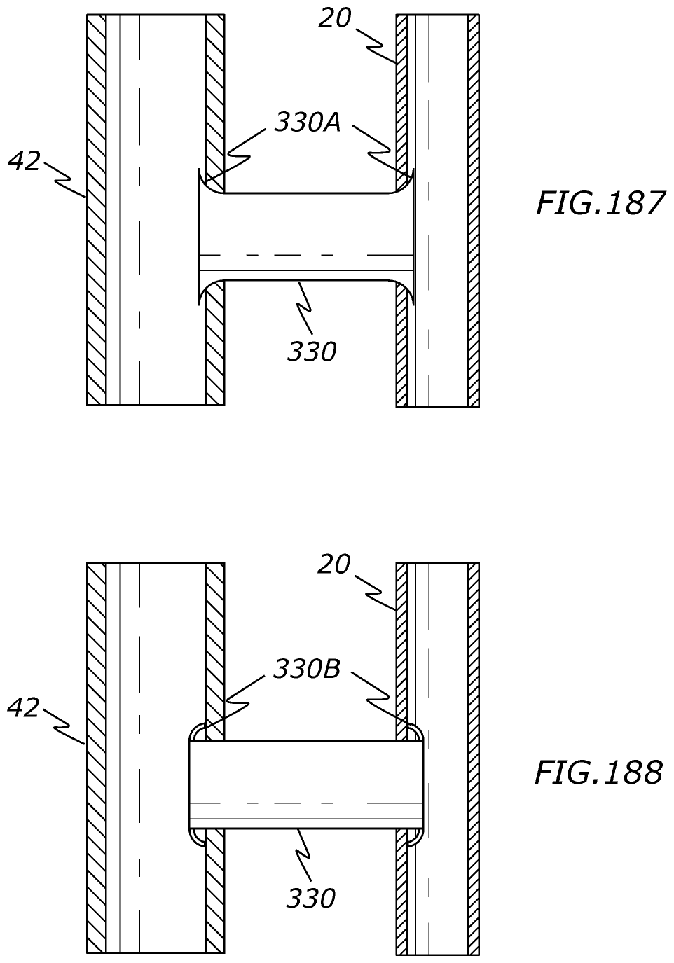

The shunt 330 may provide a means of establishing a fluid path from the lymphatic system to the Azygos vein 42 (or other vessel in the venous system or other body lumen). The shunt 330 may be covered with a material that is impermeable to blood such as polyethylene or polyurethane. The shunt 330 may be comprised of a metallic material able to maintain its shape after deployment such as nitinol or stainless steel, platinum, tantalum, or cobalt chromium. The shunt 330 may be self-expanding or expanded to a final size by another device such as a balloon. The shunt 330 may have a larger outer diameter than the hole created in each vessel so there is a compressive force applied by the vessel onto the shunt to create a friction force to maintain the position of the shunt 330. The shunt may contain retention features to maintain its position in the lymphatic and venous systems. These features may include radially flared ends 330A that expand to a diameter larger than the apertures through the venous and lymphatic vessels as seen in FIG. 187, or hooks 330B on each end of the shunt 330 as seen in FIG. 188, to directly engage the tissue.

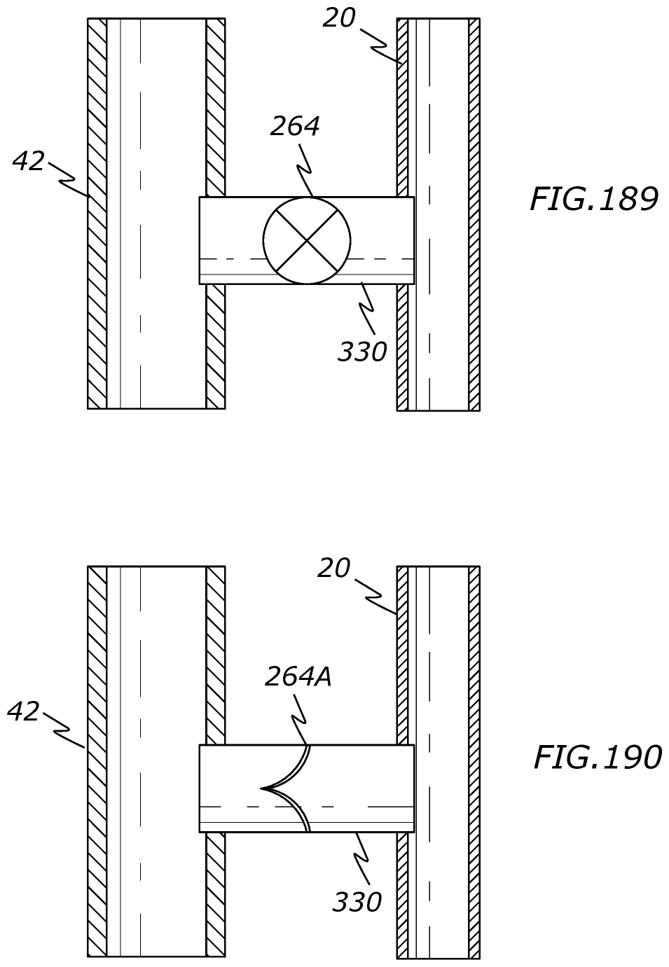

As seen in FIG. 189, the shunt 330 may contain a valve 264 to restrict flow to only one direction, such as from the lymphatic system (thoracic duct 20) to the venous system (e.g., Azygos vein 42). This may prevent fluid flow from the venous system (e.g. blood) into the lymphatic system. The valve 264 may be pressure-controlled so it only opens when a sufficient pressure difference is present across the valve 264 such as 0 mmHg, 5 mmHg, or more than 10 mmHg.

In one example seen in FIG. 190, a leaflet valve 264A including leaflets may be used to allow uni-directional flow. The leaflets may be comprised of a stiff material or composite material that would only open with a sufficient amount of pressure.

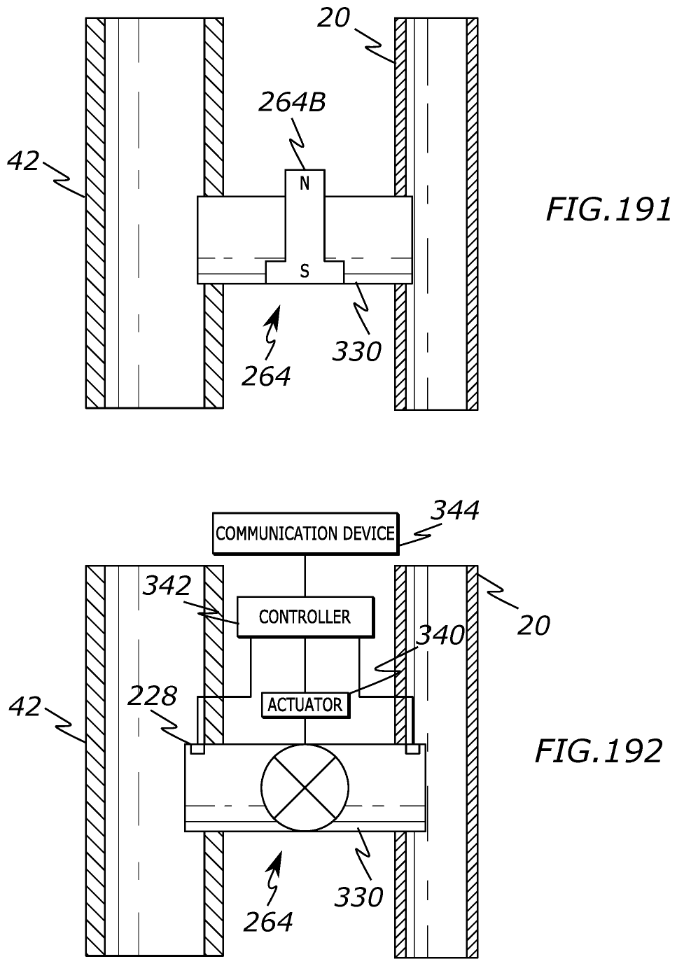

In another embodiment seen in FIG. 191, a valve 264 may also be opened by a magnetic field. The valve 264 may include a valve member 264B that can be moved by application of a magnetic field, for example, by including a magnet or ferrous metal. The patient or clinician may control the opening and closing of the valve to control when and how much fluid is drained by placing a magnet or similar magnetic device over the skin near the shunt 330 to open the valve 264 and allow drainage. The patient or clinician may close the valve by application of a magnetic field, such as by flipping the magnet over to reverse the polarity or by moving a magnetic device in a different direction.

Alternatively, as seen in FIG. 192, a shunt system may be electronically actuated to open and close. The shunt system may include a shunt 330 with one or more valves 264, a controller 342, one or more actuators 340, one or more sensors 228, and a communication device 344. The controller 342 may include a processor or similar circuitry that is connected to control the valve actuator 340, receive data from the sensors 228, and send and receive data (including command data) with the communication device 344.

The controller 342 may be configured to activate the actuator 340 to open the valve 264 in the presence of a desired stimulus sensed with the one or more of the sensors 228 (e.g. elevated pressure in the lymphatic system or pressure gradient across the shunt 330). The valve 264 may be programmed to close in the presence of another stimulus (e.g. reduced pressure threshold in the lymphatic system or reduced pressure gradient across the shunt). The controller 342 may also employ the communication device 344 to transfer the sensor readings to an external device (such as a cell phone using a Bluetooth or wireless connection protocol) to notify a physician of the sensor readings. The physician may then remotely and wirelessly communicate with the controller 342 and actuate the valve 264 to initiate/terminate drainage. The system may be powered wirelessly or with an internal battery.

It should be noted that these example uses of the Azygos vein 42 are for illustrative purposes only and other vessels in the venous system may be used such as the vena cava, subclavian vein, internal jugular vein, brachiocephalic vein, or external jugular vein. Additionally, other body structures outside of the venous system may be the destination of the shunt such as the digestive system (e.g. esophagus, stomach, small intestine, large intestine, colon) or urinary system (e.g. bladder, ureter, urethra). The digestive system may be advantageous due to its ability to resorb some or all of the constituents of the drained lymphatic fluid. The urinary system may be advantageous due to its ability to quickly excrete the fluid and eliminate from the body.

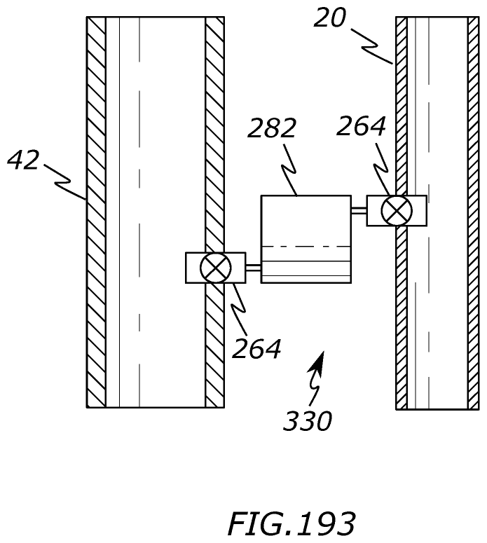

In another embodiment shown in FIG. 193, a shunt 330 may including a reservoir 282. Each end of the shunt 330 may have one or more valves 264 that restrict flow into and out of the reservoir 282 in only one direction. The valves 264 may also be electronically controlled in a manner similar to that shown in FIG. 192 (controller 342, actuator 340, sensor 228, and communication device 344) to better control flow into and out of the reservoir 282. The shunt 330 may be deployed between the Azygos vein 42 and thoracic duct 20 in one example, or between the cisterna chyli 26 and bladder of a patient in another example. When the pressure in the lymphatic system exceeds a threshold, the valve 264 in the inlet conduit may open to allow flow into the reservoir 282. When the pressure falls below the same or another threshold, the valve 264 may close. When the reservoir 282 is full or exceeds a predetermined volume, the valve 264 in the outlet conduit may open to allow flow out of the reservoir 282 into the bladder or venous vessel, for example. Alternatively, the patient may be notified to by the system that the reservoir 282 is full and the patient may open the valve in the outlet conduit to drain the reservoir 282 at a convenient time and in a discrete location.

In some embodiments, one or more sensors may be used to measure one or more physical, electrical, physiologic or other signals in the body to determine the appropriate time to initiate a drainage event, increase or decrease the flow rate of the lymphatic fluid, or terminate a drainage event.

One or more of the following signals may be measured and used to optimize the care of a patient including but not limited to the salinity of the lymphatic fluid, the pH of the lymphatic fluid, the oncotic pressure of the lymphatic fluid, color of lymphatic fluid, respiration rate, blood pressure, heart rate, diameter of a lymphatic vessel, and impedance of the lymphatic fluid.

One or more of the following signals may be used to signify that a drainage event is needed including but not limited to signs of physical congestion (edema, dyspnea, orthopnea, difficulty with exercising), elevated interstitial pressure (e.g. pressure >0 mmHg), impaired kidney function (e.g. eGFR <90 mL/min/1.73 m²), reduced urine output (e.g. <500mL in 24hr), acute drop in urine output relative to previous readings, elevated thoracic duct pressure (e.g. mean pressure >10 mmHg), elevated central venous pressure (e.g. CVP>15 mmHg), reduced or negative difference between the maximum thoracic duct and the central venous pressure (e.g. maximum thoracic duct pressure—central venous pressure <0 mmHg), engorged thoracic duct (e.g. diameter>5 mm), increase in body weight, distension of veins under visual observation or ultrasound evaluation (e.g. internal jugular, external jugular, vena cava, subclavian, brachiocephalic, renal vein), the presence of B-Lines on ultrasound of the lungs, thickening of the pleural line on ultrasound, elevated amount of lung water as detected by radar, elevated pressures in the heart (e.g. right atrial pressure, left atrial pressure, right ventricular pressure, left ventricular pressure, pulmonary artery pressure, or pulmonary vein pressure), decrease in oxygen saturation (e.g. SpO2<95%), increase in intraabdominal pressure (e.g. IAP>12 mmHg), increase in bladder pressure (e.g. bladder pressure>10 mmHg), detection of worsening heart function (e.g. using ambulatory pulmonary blood pressure measurements), abnormal salinity of lymphatic fluid, abnormal concentration of fluid constituents in blood or lymphatic fluid (e.g. white blood cells, leukocytes, protein, and water). The one or more signals may be measured, interpreted, and acted upon individually, as a group, in relative relationship to one other (e.g. one signal greater than another signal), or in a mathematical relationship to one another (e.g. one signal minus another signal).

The patient may be monitored during drainage to indicate the patient's response to the treatment and guide changes in any drainage parameters including but not limited to height of drainage container, presence and amount of suction pressure applied to drainage catheter, size of drainage device (i.e. catheter or needle), location of distal tip of drainage device if applicable, and patient orientation (i.e. lying flat, lying on one side, standing up, or sitting) to increase or decrease the amount and rate of fluid removal from the patient.

Changes in one or more of the following signals may be used to determine whether a change in therapy is required including but not limited to limited to signs of physical congestion (edema, dyspnea, orthopnea, difficulty with exercising), interstitial pressure, kidney function, urine output, thoracic duct pressure, central venous pressure, thoracic duct size (i.e. diameter), body weight, distension of veins (e.g. internal jugular, external jugular, vena cava, subclavian, brachiocephalic, renal vein), the presence or absence of B-Lines on ultrasound of the lungs, thickness of the pleural line on ultrasound, amount of lung water as detected by radar, pressures in the heart (e.g. right atrial pressure, left atrial pressure, right ventricular pressure, left ventricular pressure, pulmonary artery pressure, pulmonary vein pressure, oxygen saturation, intraabdominal pressure, bladder pressure, heart function (e.g. using ambulatory pulmonary blood pressure measurements). The one or more signals may be measured, interpreted, and acted upon individually, as a group, in relative relationship to one other (e.g. one signal greater than another signal), or in a mathematical relationship to one another (e.g. one signal minus another signal).

One or more of the following signals may be used to signify that a drainage event is complete or should be terminated including but not limited to partial or full resolution of symptoms of physical congestion (edema, dyspnea, orthopnea, difficulty with exercising), reduced interstitial pressure (e.g. pressure<0 mmHg), improved or normal kidney function, increased or normal urine output (e.g. >500 mL in 24 hr), acute increase in urine output relative to previous readings, reduced or normal thoracic duct pressure (e.g. mean pressure <10 mmHg), reduced or normal central venous pressure (e.g. CVP <15 mmHg), increased, normal, or positive difference between the maximum thoracic duct and the central venous pressure (e.g. maximum thoracic duct pressure−central venous pressure>0 mmHg), normalization of thoracic duct size (e.g. diameter<5 mm), reduction in body weight, reduction in distention of veins (e.g. internal jugular, external jugular, vena cava, subclavian, brachiocephalic, renal vein), the absence of B-Lines on ultrasound of the lungs, thinning of the pleural line on ultrasound, reduced or normal amount of lung water as detected by radar, reduced or normal pressures in the heart (e.g. right atrial pressure, left atrial pressure, right ventricular pressure, left ventricular pressure, pulmonary artery pressure, pulmonary vein pressure, increased or normal oxygen saturation (e.g. SpO2<95%), reduced or normal intraabdominal pressure (e.g. IAP<12 mmHg), reduced or normal bladder pressure (e.g. bladder pressure<10 mmHg), improved or normal heart function (e.g. using ambulatory pulmonary blood pressure measurements), volume of fluid removed, normalization of fluid constituent concentrations (e.g. white blood cells, leukocytes, protein, and water), reduction in fluid removal flow rate without change in drainage parameters such as height of drainage container, presence and amount of suction pressure applied to drainage catheter, size of drainage device (i.e. catheter or needle), location of distal tip of drainage device if applicable, and patient orientation (i.e. lying flat, lying on one side, standing up, or sitting). The one or more signals may be measured, interpreted, and acted upon individually, as a group, in relative relationship to one other (e.g. one signal greater than another signal), or in a mathematical relationship to one another (e.g. one signal minus another signal).

One or more of the above signals may be measured by a sensor in a system as described elsewhere in this application. The one or more signals may be transmitted to a controller as previously discussed in this specification for processing and transmission to a communications device. The information transmitted via the communications device may be transmitted to a cell phone, output monitor, LCD screen, or other display means. The patient or physician may use this transmitted information to start or stop a therapy session. The patient or physician may use this transmitted information to increase or decrease one or more drainage parameters including but not limited to target lymphatic structure pressure, target lymphatic fluid drainage flow rate, target lymphatic fluid volume removal, suction pressure applied to the drainage device, and duration of drainage session. The physician may use this transmitted information to adjust a medication of a patient such as a diuretic medication. Alternatively, after the controller processes the one or more signals from the sensors, one or more actuators may be activated to start, stop, or adjust a drainage therapy session as mentioned elsewhere in this application. The one or more actuators may include a solenoid or a piston.

One or more of the disclosed devices, systems, or methods may be used to diagnose, treat, ameliorate, minimize, or prevent one or more of many disease states and symptoms including but not limited to heart failure, acute decompensated heart failure, circulatory congestion, pulmonary congestion, sepsis, pulmonary edema, peripheral edema, dyspnea, orthopnea, bendopnea, elevated cardiac filling pressures, renal edema, portal edema, hypertension, renal hypertension, portal hypertension, exercise intolerance, severe acute pancreatitis, chylothorax, lymphorrhea, lymphocele, chylous effusion, ascites, organ rejection, and weight gain.

The following embodiments are directed to various aspects of directly enhancing fluid flow through the lymphatic system of a patient.

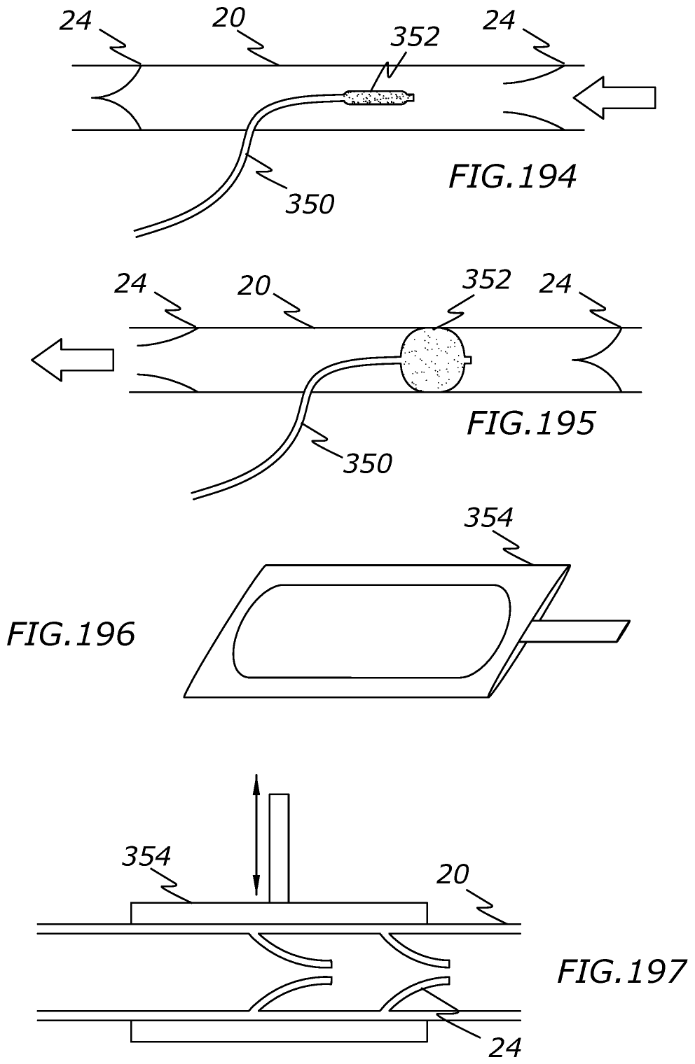

In one embodiment and method seen in FIGS. 194 and 195, a distal end of a balloon catheter 350 may be placed in a lymphatic vessel (e.g., thoracic duct 20) between two valves 24. A balloon 252 on the distal portion of the catheter 350 may be inflated to increase the pressure in the space between the valves, leading to forward flow through the distal valve 24. The balloon 352 then may be deflated to reduce the pressure in the space between the valves 24 and allow flow into the space through the proximal valve 24. The balloon 352 may be cycled between inflated and deflated states to enhance the flow through the lymphatic vessel. The balloon 352 may be inflated using gas or fluid (e.g., saline). Since the end of the thoracic duct 20 typically includes a plurality of valves 24, this location may be a particularly effective location to enhance flow from the thoracic duct into the venous system of a patient.

In one embodiment and method shown in FIGS. 196 and 197, an external cuff 354 may be placed around a lymphatic structure (e.g., a thoracic duct 20). The cuff 354 may be a miniaturized blood pressure cuff that may radially compress a lymphatic structure. For example, the cuff 354 may but a tubular shape that inflates with a gas or fluid (cuff 354 is shown in an unrolled state in FIG. 196). Alternatively, the cuff 354 may be a linear clamp that may be translated to compress a lymphatic structure. Actuating the cuff 354 to compress a lymphatic structure may increase the internal pressure to enhance forward flow through distal valves 24. The cuff 354 may be relaxed to decrease the internal pressure in the region of the cuff 354 to enhance flow into the space from the proximal end of the lymphatic structure. The cuff 354 may be cycled between actuated and relaxed states to enhance the flow through the lymphatic vessel.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method of medical treatment comprising:
advancing a drainage device into a patient;
draining fluid from a lymphatic system of the patient. cm 2. The method of claim 1, wherein advancing a drainage device into a patient further comprises advancing a needle into the lymphatic system of the patient. cm 3. The method of claim 2, wherein advancing a drainage device into a patient further comprises advancing a needle into a lymph node, a thoracic duct, a cervical portion of the thoracic duct, a thoracic portion of the thoracic duct, a right thoracic duct, a renal lymphatic vessel, and/or a cisterna chyli of the patient. cm 4. The method of claim 2, further comprising confirming the needle has accessed the lymphatic system. cm 5. The method of claim 4, wherein confirming the needle has accessed the lymphatic system includes identifying at least one of color, pH, viscosity, impedance, salinity, and absence of red blood cells of the fluid that has been drained. cm 6. The method of claim 4, wherein confirming the needle has accessed the lymphatic system includes receiving sensor data from one or more sensors included with the needle; wherein the one or more sensors are configured to measure color, pH, viscosity, impedance, salinity, pressure, orientation, compliance, or size of a lymphatic structure. cm 7. The method of claim 4, wherein confirming the needle has accessed the lymphatic system includes injecting a contrast agent through the needle and imaging the patient to view the contrast agent. cm 8. The method of claim 4, wherein confirming the needle has accessed the lymphatic system comprises injecting a contrast agent into a lymphatic structure of the lymphatic system; visualizing the contrast agent; and advancing the needle into the lymphatic system. cm 9. The method of claim 2, further comprising advancing a guidewire through the needle; removing the needle from the patient; and advancing a catheter over the guidewire and into the lymphatic system. cm 10. The method of claim 1, wherein advancing a drainage device into a patient further comprises imaging a patient with ultrasound, CT, or MRI. cm 11. The method of claim 2, wherein the needle is composed of echogenic material configured to be visualized via ultrasound. cm 12. The method of claim 1, wherein advancing a drainage device into a patient further comprises advancing a distal end of a catheter into an upper cervical portion of a thoracic duct, a thoracic portion of the thoracic duct, or a cisterna chyli. cm 13. The method of claim 1, wherein advancing a drainage device into a patient further comprises advancing a distal end of a catheter into a lymphatic structure. cm 14. The method of claim 13, wherein advancing the distal end of the catheter into a lymphatic structure further comprises radially expanding the distal end of the catheter within the lymphatic structure. cm 15. The method of claim 14, wherein expanding the distal end of the catheter further comprises expanding the distal end to a conical shape, a curved shape, a square shape, a bent shape, or a coiled shape. cm 16. The method of claim 13, wherein advancing the distal end of the catheter into a lymphatic structure further comprises delivering a stent within the lymphatic structure and connecting the distal end of the catheter to the stent. cm 17. The method of claim 16, wherein connecting the distal end of the catheter to the stent is performed by magnets or hooks. cm 18. The method of claim 13, wherein advancing the distal end of the catheter into a lymphatic structure further comprises deploying a stent from the catheter;
maintaining a connection between the stent and the catheter during drainage, and detaching the catheter from the stent. cm 19. The method of claim 13, wherein advancing the distal end of the catheter into a lymphatic structure further comprises applying adhesive at least partially around an interface between the lymphatic structure and the catheter. cm 20. The method of claim 13, wherein advancing the distal end of the catheter into a lymphatic structure further comprises advancing adhesive through a lumen in the catheter and applying the adhesive to the lymphatic structure and the catheter.

21-203. (canceled)
